(19) United States
(12) Patent Application Publication
(10) Pub. No.: US 2025/0019690 A1
(43) Pub. Date: Jan. 16, 2025

(54) ULTRA-HIGH RESOLUTION MAPPING OF 3D GENOME STRUCTURE USING REGION CAPTURE MICRO-C

(71) Applicant: Massachusetts Institute of Technology, Cambridge, MA (US)

(72) Inventors: Anders Sejr Hansen, Cambridge, MA (US); Viraat Y. Goel, Somerville, MA (US); Miles Huseyin, Cambridge, MA (US)

(21) Appl. No.: 18/773,403

(22) Filed: Jul. 15, 2024

Related U.S. Application Data
(60) Provisional application No. 63/513,510, filed on Jul. 13, 2023.

(51) Int. Cl. C12N 15/10; C12N 9/22

(52) U.S. Cl. CPC C12N 15/1082; C12N 9/22; C12N 15/1006

(57) ABSTRACT

A method for identifying nucleic acid regions which interact with each other in genomic DNA, the method including the steps of: (1) crosslinking genomic DNA (including chromatin) in cells; (2) digesting the crosslinked genomic DNA to obtain fragments; (3) fragment end labelling; (4) ligating nucleic acid fragments; (5) obtaining a purified DNA fraction of ligation products including mainly di-nucleosomes; (6) preparing a sequencing library from the DNA fraction of ligation products mainly di-nucleosomes; and (7) performing tiling region capture of a region of interest.

Selection of processing steps/reagents in the disclosed method provides a significantly improved method when compared to prior methods such as Hi-C, Micro-C, Micro-Capture-C (MCC), and Tiled-Micro-Capture-C (TMCC) in terms of reduction in cost, significantly improved efficiency, with capture of genomic interactions including enhancer-promoter interactions such as microcompartments.

### CROSS-REFERENCE TO RELATED APPLICATION

This application claims the benefit of and priority to U.S. Provisional Application No. 63/513,510 filed Jul. 13, 2023, the entire content of which is incorporated herein by reference for all purpose in its entirety.

### STATEMENT REGARDING FEDERALLY SPONSORED RESEARCH

This invention was made with government support under grant numbers DP2-GM140938-01, UM1-HG011536 and R33-CA257878 awarded by National Institute of Health, grant number MCB-2036037 awarded by National Science Foundation, and grant number P30-CA14051 awarded by National Cancer Institute. The U.S. government has certain rights in this invention.

### FIELD OF THE INVENTION

This invention is generally in the field of Chromosome Conformation Capture.

### BACKGROUND OF THE INVENTION

Chromosomes and genomes are generally believed to be organized in three dimensions (3D) such that functionally related genomic elements, e.g., silencers and enhancers and their target genes, are directly interacting or are located far away from each other. 3D genome structure regulates vital cellular processes including gene expression, DNA repair, genome integrity, DNA replication, and somatic recombination^{1,2}. Many insights into 3D genome structure have come from Chromosome Conformation Capture (3C) assays, which have revealed structural hallmarks across at least three scales. First, active and inactive chromatin segregate into A- and B-compartments through a poorly understood compartmentalization mechanism^{3,4}. Second, the genome is folded into loops and local domains called Topologically Associating Domains (TADs) or loop domains⁵⁻⁸ by loop-extruding cohesin complexes halted at CTCF boundaries^{9,10}. Third, while A/B-compartments and TADs generally span hundreds to thousands of kilobases, recent work has hinted at finer scale 3D chromatin interactions including between enhancers and promoters¹¹⁻¹⁷. Because enhancers are the primary units of gene expression control in mammals, there has been intense interest in resolving fine-scale enhancer-promoter (E-P) interactions. However, it has remained challenging to resolve fine-scale E-P interactions with current methods^{8,18}.

Advances in the understanding of 3D genome structure have been primarily driven by: (1) deeper sequencing; (2) improved 3C protocols; and (3) perturbation studies. First, A/B-compartments, TADs, and loops were uncovered as deeper sequencing increased the number of captured unique contacts in 3C experiments from ˜8 million³ to ˜450 million⁵ to ˜5 billion⁷, respectively. Second, in overcoming the resolution limits imposed by Hi-C's dependence on restriction enzymes, Micro-C achieved nucleosome-scale resolution by digesting chromatin with micrococcal nuclease (MNase); this allows Micro-C to better resolve finer-scale regulatory interactions including between enhancers and promoters^{8,11-13,15,19,20}. Third, perturbation studies have yielded mechanistic insights into 3D genome structure. For example, protein-depletion studies were pivotal in elucidating the roles of CTCF, cohesin, and associated factors in the formation of TADs and loops^{12,21-27}.

Nevertheless, sequencing costs remains the key bottleneck for 3C assays. For a genome with n bins, sequencing costs to populate an n² pairwise contact matrix grow quadratically with n. For example, approximately $1.6 billion in sequencing costs alone are needed to average one read per nucleosome-sized bin across the human genome (a total of (3.3×10⁹ bp/150 bp) 2/2=2.4×10¹⁴ reads).

There is therefore a need to provide an improved method for identifying 3D interactions within DNA regions which overcomes the limitations of the currently available methodologies.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

A method is provided for identifying DNA regions which interact with each other in genomic DNA; the method includes the steps of: (1) crosslinking genomic DNA (including chromatin) in cells; (2) digesting the crosslinked genomic DNA to obtain fragments which are predominantly mono-nucleosomes; (3) repairing the fragment ends, followed by fragment end labelling; (4) ligating nucleic acid fragments; (5) obtaining purified ligation products which include ligated di-nucleosomes; (6) preparing a sequencing library from the ligation products; and (7) performing tiling region capture of a region of interest. The cell sample size introduced into the crosslinking step should generate sample with a sufficient range of fragments to cover the genomic region, for example, more than 5 million (5M) Cells.

The chromatin in cells is crosslinked using a suitable crosslinking agent, preferably at least two crosslinking agents, preferably, in a single step including sequential crosslinking (i.e., double crosslinking), prior to quenching of the crosslinking reaction. Preferred crosslinking agents include disuccinimidyl glutarate (DSG) and formaldehyde. In some forms, cells are counted following crosslinking to quantify losses during cell crosslinking and collection steps. This helps ensures that the ratio of reagents (e.g., MNase) to cell number is consistent during subsequent steps.

Digesting the crosslinked chromatin to obtain nucleosome-sized fragments (150-200 bp) is performed using any suitable nuclease, more preferably using micrococcal nuclease (MNase), by contacting a cell composition with an MNase composition in an amount and a time effective for digestion of the chromatin. If the cells are from a frozen sample, the cells are thawed and resuspended in a buffer composition, in some forms containing bovine serum albumin (BSA; at about 100 μg/mL), for improved pelletization. The method used to obtain nucleosome-sized fragments preferably does not include sonication.

The nucleosome-sized fragments are end-labelled following fragment end repair, using as a label an agent that has a binding partner (of a binding pair) to which it binds by affinity binding. An example of a labelling agent is biotin. Thus, the nucleosome-sized fragments are end-labelled through incorporation of a pool of biotin-labelled nucleotides.

In some forms, a reverse crosslinking step is included in the steps to obtains a purified DNA fraction, and it includes adding NaCl (about 200 mM) reverse crosslinking reaction mix to improve reverse crosslinking efficiency and increase DNA yield. In some forms, the reaction step of reversing crosslinking include NaCl, at a concentration of between about 10 mM and about 500 mM, preferably between about 100 mM and about 300 mM, more preferably, between 200 mM and about 250 mM, inclusive. In some forms, the disclosed methods do not include an ethanol precipitation (of DNA) step.

In some forms, a purified DNA fraction is obtained in the size range of ˜200-350 bp, which contains predominantly ligated di-nucleosomes as well as other ligation products including footprints from a reverse-crosslinked sample, by isolating DNA fragments in the size range of ˜200-350 bp using a suitable method, for example, using gel electrophoresis (subjecting the sample to gel electrophoresis; for example, using 1% agarose gel and extracting di-nucleosomal DNA from the 1% agarose gel). In some forms, ligated DNA contact fragments are isolated by pulling down label-bound fragments using a binding partner for the label. For example, where the label is biotin, streptavidin can be used to pull down biotin-bound fragments.

In some forms, the stage of library preparation includes one or more steps of: (i) end polishing, (ii) streptavidin purification, (iii) end repair & A-tailing, (iv) adapter ligation, (vi) bead washing, (vii) test PCR run, (ix) pool PCR run, (x) sample purification, (xi) sample quantification, and (xii) pooling of barcoded samples.

In some forms, tiling region capture comprises adding labelled capture probes, wherein the labelled capture probes hybridize to regulatory regions of the crosslinked genomic DNA, and the labelled capture probes are about 80 bases long and tile regions around about a hundred kilobases to several megabases in the genomic DNA; selectively purifying the fragments that hybridize to the labelled capture probes; and analyzing the fragments that hybridize to the labelled capture probes to determine the identity and 3D interactions of the fragments.

In preferred embodiments, the method identifies enhancer-promoter interactions, DNA 3D interactions and/or microcompartments.

The disclosed methods provide improved evaluation and detection of direct intra- and inter-chromosomal interactions between remote regulatory elements; this information can be used to diagnose specific medical and/or biological conditions. In particular, the disclosed methods may be applied to uncover the gene targets and likely functions of disease-associated genetic variants discovered through, for example, genome-wide association studies.

### BRIEF DESCRIPTION OF THE DRAWINGS

FIGS. 1A and 1B shows that RCMC captures chromosome conformation at unprecedented resolution, far exceeding previous methods. FIG. 1A is a schematic overview of the RCMC protocol. Cells are chemically fixed, nuclei are digested with MNase, and fragments are biotinylated, proximity-ligated, di-nucleosomes gel-extracted and purified, library-prepped, PCR-amplified, and region-captured to create a sequencing library. After sequencing, mapping and normalization, the data are visualized as a contact matrix. FIG. 1B is a dot plot showing benchmarking comparison of RCMC against the highest-resolution Tiled-Micro-Capture-C (TMCC)¹⁷, Micro-C¹² and Hi-C³¹ mouse embryonic stem cell (mESC) datasets. Region-averaged calculations are shown for RCMC, TMCC, Micro-C and Hi-C, and calculations for individual captured regions are also shown for RCMC and TMCC. The x axis shows the fraction of all reads that uniquely map to the target region (both read mates fall within the captured region) that are structurally informative (defined as cis contacts ≥1 kb). The y axis shows the fraction of all contact bins separated by 10 kb that contain at least one read at 100-bp resolution.

FIGS. 2A-2C shows that RCMC generates deep contact maps, reveals previously unresolved aspects of 3D genome structure, and outperforms other 3C methods. FIGS. 2A and 2B are contact map comparison of RCMC against the deepest available mESC Hi-C (top; Bonev et al.³¹) and Micro-C (middle; Hsich et al.¹²) datasets at the mouse Sox2 (FIG. 2A) and Klf1 (FIG. 2B) regions at 500-bp resolution. Gene annotations and ATAC, chromatin immunoprecipitation with sequencing (ChIP-seq) and RNA sequencing (RNA-seq) (Table 18) signal tracks are shown below the contact maps, whereas the contact intensity scale is shown to the right. The RCMC data shown were pooled from two biological replicates in wild type (WT) mESCs. FIG. 2C is a contact map comparison of RCMC against TMCC¹⁷ at the Nanog locus at 250-bp resolution. Full datasets are visualized in the top contact map, and TMCC has been downsampled to match the total number of RCMC sequencing reads in view in the bottom contact map.

FIGS. 3A-3G shows that RCMC identifies highly nested focal interactions called microcompartments, which frequently connect enhancers and promoters. FIGS. 3A and 3B are contact map visualization of RCMC data and called microcompartments at the Klf1 (FIG. 3A) and Ppm1g (FIG. 3B) loci at 500-bp (FIG. 3A) and 1-kb (FIG. 3B) resolution (left) and 250-bp resolution (zoom in, right). Manually annotated microcompartment contacts are shown below the contact map diagonal on the left, whereas comparisons against genome-wide Micro-C¹² (FIG. 3A) and Hi-C³¹ (FIG. 3B) are shown on the right. FIGS. 3C and 3D are histograms showing distributions of the number of focal interactions formed by microcompartment anchors (FIG. 3C) and the lengths spanned by focal interactions in kilobases (FIG. 3D). FIG. 3E is a Venn diagram of microcompartment anchor categories according to chromatin features overlapped by the anchor ±1 kb. Promoters were defined as regions around annotated transcription start sites⁵¹±2 kb, active enhancers as regions with overlapping peaks of H3K4mel (ENCFF282RLA) and H3K27ac (GSE90893) in ChIP-seq data that did not overlap promoters, and CTCF/cohesin as regions with overlapping peaks of CTCF (GSE90994) and SMC1A (GSE123636) in ChIP-seq data. Other regions are those not overlapping any of these features. FIG. 3F is a swarm plot of the number of focal interactions formed by individual microcompartment anchors divided according to categories in panel FIG. 3E, including the mean (u) and median (Med) for each distribution. Anchors fitting into more than one category were excluded. FIG. 3G is a pie chart showing fractions of loops classified into different categories: P-P (promoter-promoter), E-P, CTCF-CTCF (CTCF/cohesin-CTCF/cohesin), and other (other-other interactions, or any other combinations). CTCF-CTCF interactions do not include any anchors that overlap promoter or enhancer regions.

FIGS. 4A-4E show that most microcompartments are robust to the loss of loop extrusion. FIG. 4A is a contact map showing that cohesin (RAD21) depletion does not strongly perturb most microcompartments. Left: Treatment paradigm for rapid depletion of RAD21 upon IAA treatment in clone FIM RAD21-mAID-BFP-V5 mESCs^{13,38}. Right: Contact maps comparing DMSO-treated control (above) and RAD21-depleted (below) samples are shown for the Klf1 and Ppm1g loci. FIG. 4B is a western blot showing near complete (97%) depletion of RAD21 following 3 h of IAA treatment. This western blot was performed once using cells collected simultaneously for RCMC. FIG. 4C is an aggregate peak analysis matrix of called microcompartmental contacts after RAD21 depletion compared to their respective controls, separated by the identity of each contact's constituent anchors. Plots show a 20-kb window centered on the loop at 250-bp resolution. The background-normalized intensity for a 1,250×1,250 bp box around the central dot for each aggregate peak is shown in the upper right of each plot as a quantification of aggregate dot strength. FIG. 4D is a plot of individual microcompartment strengths (as quantified in panel FIG. 4C) in the RAD21-depleted (y axis) and control (x axis) conditions, shown for P-P (purple, n=418), E-P (pink, n=238) and E-E (gray, n=40) loops. Interactions changing in strength by two-fold, or more are visualized as x's with percentages noted, whereas interactions below the threshold are visualized as circles,. FIG. 4E are zoomed-in contact maps of microcompartment examples in panel FIG. 4A that strengthen (i) or weaken (ii, iii) relative to the control treatment and the background in response to RAD21 depletion.

FIGS. 5A-5F show that most microcompartments are robust to the inhibition of transcription. FIG. 5A shows inhibition of transcription initiation with triptolide does not strongly affect most microcompartments. Left: Overview of triptolide treatment for WT mESCs (45 min or 4 h). Right: Contact maps comparing WT control (above) and transcriptionally inhibited (below) samples are shown for the Klf1 (45-min timepoint shown vs. control) and Ppm1g loci (4-hr timepoint shown vs. control). RNA Pol II ChIP-seq data (RPB1) are shown below. FIG. 5B is an aggregate RPB1 RNA Pol II ChIP-seq signal at genes after triptolide treatment (45 min and 4 h) and a control (WT). The x axis depicts all unique mouse genes normalized by length and flanked by 3 kb upstream and downstream of their transcription start site (TSS) and transcription end site (TES), respectively. The first 500 bp downstream of the TSS (marked by the second x axis tick mark) are not normalized to avoid normalizing the core promoter against variable gene body lengths. FIG. 5C shows contact maps comparing the transcriptional inhibition timepoints (45 min treatment above, 4 h treatment below) for the Klf1 locus (left), and zoomed-in contact maps of microcompartments across the control and triptolide treatment timepoints that weaken (i) or strengthen (ii, iii) in response to transcriptional inhibition (right). FIG. 5D shows plots of individual microcompartment strengths in the transcriptionally inhibited (y axis) and control (x axis) conditions, shown for P-P (purple, n=418), E-P (pink, n=238) and E-E (gray, n=40) loops. Interactions changing in strength by two-fold, or more are visualized as x's (percentages noted), and as circles otherwise. FIG. 5E is an aggregate peak analysis matrix of called microcompartmental contacts across the two transcriptional inhibition timepoints compared to the control, separated by the identity of each contact's constituent anchors. Plots show a 20-kb window centered on the loop at 250-bp resolution, with background-normalized dot intensities shown in the upper right of each plot. FIG. 5F is a schematic for the proposed model for the formation of microcompartments. Coalescence of multiple promoters and enhancer elements in a gene-dense region may occur through A/B-block copolymer microphase separation, resulting in variable combinations of multiway interactions being present in different cells and giving rise to tessellated focal interactions in population-averaged RCMC data.

FIGS. 6A-6G show that RCMC efficiently and reproducibly captures ligated di-nucleosomal fragments, giving rise to deep contact maps. FIG. 6A is a representative MNasc titration DNA gel indicating the ideal level of digestion by MNase, based on the ratio of fragment sizes, for the RCMC protocol. FIG. 6B is a representative size-selection gel for the RCMC protocol showing the 200-350 bp band that is extracted to obtain ligated fragments. FIG. 6C is a schematic overview of the capture probe design workflow for RCMC. 80-mer probes tiling the region of interest are designed, removing those which overlap highly repetitive regions. FIG. 6D is a summary of the capture efficiency for each of the five regions for which probes were designed. The locations and sizes of the regions, the number of ligated fragments which mapped at single loci at both ends in total and in the region, and the capture efficiencies are given. Because different capture probe sets were used for Biological Replicates 1 (two separate sets of capture probes) and 2 (simultaneous capture for all five loci), numbers are separately provided for each Biological Replicate. FIG. 6E shows contact maps comparing raw, unbalanced data (upper panel, lower triangle), ICE-balanced³⁰ to all aligned reads (lower panel, lower triangle) and ICE-balanced to reads in captured loci only (both panels, upper triangle). Balancing only to data entirely within captured loci was necessary to remove artifacts due to capture bias. FIG. 6F compares contact maps of the entire Fbn2 TAD in RCMC and in Hi-C³¹ and Micro-C¹². Gene annotations and ChIP-seq signal tracks are shown below the contact maps. FIG. 6G is a measurement of reproducibility between WT replicates across all five capture loci, with reproducibility scores determined using HiCRep⁵⁸ at 10 kb resolution, clustered according to similarity. Three technical RCMC replicates (denoted by ‘TR #’) comprise Biological Replicate 1, while ‘BR2’ denotes Biological Replicate 2. TR3_WT is noted in different text at the Sox2 and Nanog loci because very little TR3_WT pre-Capture library remained for input to Sox2 & Nanog capture after the initial Ppm1g, Klf1, and Fbn2 capture experiment; accordingly, relative to all other replicates, TR3_WT has much lower sequencing depth (0.5-2.4% the number of unique contacts) at the Sox2 & Nanog loci.

FIGS. 7A-7F show benchmarking of RCMC against other 3C methods. FIG. 7A are contact probability curves comparing RCMC against the highest resolution Tiled-Micro-Capture-C (TMCC)¹⁷, Micro-C¹², and Hi-C³¹ mESC datasets across contact distances. FIG. 7B is a dot plot of the benchmarking comparison of RCMC's ability to fill out high-resolution contact matrices against TMCC¹⁷, Micro-C¹², and Hi-C³¹. Region-averaged calculations are shown for all methods, and calculations for individual captured regions are also shown for RCMC and TMCC. The x axis shows the contact distance in bp, and the y axis shows the fraction of all bins at a given contact distance within the captured locus that contain at least one read at 100 bp resolution. FIG. 7C is a table summary of read counts across RCMC, TMCC¹⁷, Micro-C¹², and Hi-C³¹. The number of mapped sequencing reads, the fraction of unique reads, and the fraction of structurally informative (defined as cis contacts ≥1 kb) unique reads are given for each method. Two versions of quantification are provided for TMCC. In black are numbers processed using the same bioinformatic pipeline as for RCMC. Capture region-specific quantifications (defined here as all reads with at least one of two read mates mapped to the locus) are also provided for all RCMC loci and the Sox2 and Nanog TMCC loci; the Oct4 and Prdm14 TMCC loci are not considered in this study. Marked with an asterisk (*) are numbers obtained using the custom TMCC-specific bioinformatic pipeline from Aljahani et al.¹⁷. Values with asterisks denote quantifications of all unique contact pairs mapped to captured loci (not filtered to be ≥1 kb in size). FIG. 7D provides contact map comparisons of RCMC data generated in this study, starting from the full dataset (topmost) and successively downsampled by orders of two down to 1/128^{th} of the data (bottommost), shown for the Klf1 locus at 500 bp resolution. FIG. 7E is a benchmarking comparison, as in FIG. 7B, of successively downsampled RCMC's ability to fill out high-resolution contact matrices against Micro-C¹² at the Klf1 locus. FIG. 7F shows contact map comparisons of 1/64^{th} and 1/128^{th} downsampled RCMC (left) against the highest-resolution available mESC Micro-C¹² (right; Hsich 2020) dataset, shown for the Klf1 locus at 500 bp resolution.

FIGS. 8A-8D show that RCMC generates deeper contact maps than other 3C methods across all five captured loci. Contact map comparisons of RCMC against the highest-resolution available mESC Hi-C³¹ (top; Bonev et al. 2017) and Micro-C¹² (bottom; Hsich et al. 2020) datasets at the Klf1 (FIG. 8A), Ppm1g (FIG. 8B), Sox2 (FIG. 8C), Nanog (FIG. 8C), and Fbn2 (FIG. 8D) loci. Full captured regions are shown for each locus at resolutions ranging from 1-5 kb, as well as Klf1 (FIG. 8A) and Ppm1g (FIG. 8B) zoom-ins at 800 and 1000 bp, respectively. Gene annotations and ATAC, ChIP-seq, and RNA-seq tracks (Table 18) are shown below the contact maps, while the contact intensity scales are shown next to the maps.

FIGS. 9A and 9B demonstrate that RCMC maps the Sox2 locus more deeply and efficiently than sister methods, uncovering previously unseen interactions. FIG. 9A is a contact map comparison of RCMC against Hi-C³¹ (top) and Micro-C¹² (bottom) at the Sox2 locus at 1.6 kb resolution. Arrows mark contacts between Sox2, the SCR, and Fxr1 not mapped by Hi-C and Micro-C. FIG. 9B is a contact map comparison of RCMC against Tiled-Micro-Capture-C¹⁷ (TMCC) across the whole TMCC-Captured locus (left, 1.6 kb resolution) and in the Sox2 and SCR regulatory cluster (right, 500 bp resolution). Full datasets are visualized in the top contact maps, and TMCC has been downsampled to match the total number of RCMC sequencing reads in view in the bottom contact maps.

FIGS. 10A-10D shows that RCMC identifies microcompartments, which are not visible in other methods and not reliably called by existing algorithms. FIGS. 10A and 10B are contact maps comparison of RCMC (top) against Hi-C³¹ (bottom, FIG. 10A) and Micro-C¹² (bottom, FIG. 10B) at the Klf1 locus at 500 and 250 bp resolutions and at the Ppm1g locus at 1000 and 250 bp resolutions. FIG. 10C shows contact maps of the Klf1 and Ppm1g loci at 1 kb resolution with loop calls by Mustache³⁷ overlaid on the bottom half of the map and compartment calls by cooltools^{59,60} shown below the map. FIG. 10D shows contact maps of the entire Klf1 (3.2 kb resolution) and Ppm1g (5 kb resolution) captured loci with manually called loops (see Methods) overlaid on the bottom halves of the maps.

FIGS. 11A-11D show that microcompartments are not artifacts resulting from incomplete ICE balancing nor chromatin accessibility. FIG. 11A contains graphs showing the comparison of ICE balancing across methods and captured loci. Distributions of the sums of ICE-balanced contact matrix rows at 250 bp resolution are shown at the Klf1, Ppm1g, Fbn2, and Sox2 loci for RCMC, Micro-C¹², and Hi-C³¹, as well as for the subset of RCMC rows containing microcompartment anchors. A sharp unimodal peak is consistent with ICE's baseline assumption that all contact matrix rows and columns must sum to the same value. FIG. 11B shows metaplots (above) and heatmaps (below) depicting ATAC signal at microcompartment anchors (left, separated by whether anchors coincide with an ATAC peak) and at all ATAC peaks in the Klf1 and Ppm1g capture loci (right, separated by whether peaks coincide with a microcompartment anchor). Signals are plotted in a 2 kb window centered on the anchor (left) or the ATAC peak (right). FIG. 11C shows RCMC contact maps at the Klf1 (left, 250 bp resolution) and Ppm1g (right, 1.6 kb resolution) loci indicating ATAC peaks that do not form microcompartments (left, magenta arrows) and a microcompartment anchor that does not coincide with an ATAC peak (right, cyan arrow). Black arrows (right) indicate microcompartmental loops involving the ATAC-negative microcompartment anchor. FIG. 11D is a Venn diagram breakdown of the overlap between all manually annotated microcompartment anchors and all ATAC peaks across the Klf1 and Ppm1g capture loci. Of 132 annotated microcompartment anchors, 12 do not coincide with ATAC peaks (cyan) while 120 do (purple, *). Of 353 called ATAC peaks, 187 do not form microcompartment anchors (magenta) while 166 do (purple, **). The apparent discrepancy of 120 microcompartment anchors being anchored by 166 ATAC peaks is due to two close ATAC peaks occasionally anchoring a single microcompartment.

FIG. 12 shows that categories of microcompartment anchors can be defined by their chromatin features. Metaplots (above) and heatmaps (below) depict ATAC, ChIP-seq, and RNA-seq (Table 18) signal at microcompartment loop anchors for classes of microcompartment anchors as defined in FIG. 3E. Features are plotted in a 2 kb window centered on the anchor.

FIGS. 13A-13E shows that cohesin depletion disrupts CTCF/cohesin loops, but generally not most microcompartmental loops. Contact maps comparing a DMSO control (above) and RAD21-depleted samples (below) are shown for the Klf1 (FIG. 13A), Ppm1g (FIG. 13B), Sox2 (FIG. 13C), Nanog (FIG. 13C), and Fbn2 (FIG. 13D) loci at resolutions spanning 800 bp-5 kb in FIM RAD21-mAID-BFP-V5 mESCs^{13,38}. Arrows mark contacts lost upon RAD21 depletion. ChIP-seq data from Hsich et al.,¹³ is shown below the maps before and after the IAA treatment (500 μM, 3 hours). Two versions of the Fbn2 locus are shown in FIG. 13D, with the left using logarithmic contact frequency scaling and the right using linear scaling. Loss of the Fbn2 loop³⁸ is most clearly seen on linear scale. FIG. 13E consists of contact probability curves comparing RAD21-depleted RCMC samples against a DMSO control (top) and RAD21-depleted Micro-C samples against a DMSO control (bottom). Arrows indicate the contact frequency ‘bump’ lost upon RAD21 depletion.

FIGS. 14A-14C shows that inhibition of transcription does not significantly alter genome organization in captured loci. Contact maps comparing control data against 45 min (top) and 4 hr (bottom) transcriptional inhibition data (from 1 μM triptolide treatments) are shown for the Klf1 (FIG. 14A), Ppm1g (FIG. 14B), Sox2 (FIG. 14C), Nanog (FIG. 14C), and Fbn2 (FIG. 14C) loci at resolutions spanning 800 bp-5 kb in WT mESCs. RNA Pol II ChIP-seq data is shown below the maps for each treatment condition.

FIGS. 15A-15D depict loci containing microcompartment-like structures visible in contact maps of ultra-deep Hi-C data in human lymphoblastoid cells from Harris et al.¹⁴. Maps were generated using Juicebox's web interface⁶¹ kindly provided by Dr. Jordan Rowley. Maps are shown at 1 kb resolution, with GM12878 gene annotations, CTCF (ENCFF364OXN) and H3K27ac (ENCFF180LKW) ChIP-seq, and RNA-seq (ENCFF604VIC) signal tracks shown below the contact maps.

FIGS. 16A-16E show that RCMC is reproducible across biological and technical replicates. Measurement of reproducibility between replicates across all five Capture loci and all five tested conditions: Sox2 (FIG. 16A), Ppm1g (FIG. 16B), Nanog (FIG. 16C), Klf1 (FIG. 16D), and Fbn2 (FIG. 16E), with reproducibility scores determined using HiCRep⁶ at 10 kb resolution and clustered according to similarity. A legend (FIG. 16F) clarifies the nomenclature used to refer to different replicates and conditions. As in FIG. 6G, TR3_WT is noted in different text at the Sox2 (FIG. 16A) and Nanog (FIG. 16C) loci because very little TR3_WT pre-Capture library remained for input to Sox2 & Nanog Capture after the initial Ppm1g, Klf1, and Fbn2 Capture experiment; accordingly, relative to all other replicates and conditions, TR3_WT is uniquely sparse at the Sox2 & Nanog loci. At the Sox2 locus (FIG. 16A), TR3_WT has 52k unique contacts whereas TR1_WT, TR2_WT, and BR2_WT have 3.6M, 7.0M, and 11.2M, respectively. At the Nanog locus (FIG. 16C), TR3_WT has 47k unique contacts whereas TR1_WT, TR2_WT, and BR2_WT have 2.0M, 3.8M, and 5.3M, respectively. Thus, the lower score for TR3 is likely due to the much lower sequencing depth.

FIG. 17 is an example of a gel extraction. The cut strayed below 200 bp for size selection of di-nucleosomal DNA.

### DETAILED DESCRIPTION OF THE INVENTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented but is to be accorded the widest scope consistent with the principles and novel features disclosed herein.

The disclosed methods reduce the prohibitive cost of sequencing inherent to current methods and facilitates the study of fine-scale 3D genome structure and enhancer-promoter interactions at ultra-high resolution, by providing a chromosome conformation capture (3C) method that significantly increases effective sequencing depth, and is cost-effective for perturbation experiments by incorporating steps that reduce sample loss, improve reaction efficiency and yield, reduce processing steps, and significantly improve capture efficiency of target regions.

The disclosed method, referred to herein as Region Capture Micro-C(RCMC), is a combination of an improved Micro-C protocol with a tiling region capture approach^{28,29} to enrich for entire regions of interest. The Examples demonstrate the use of RCMC to generate the deepest maps of 3D genome organization reported so far, achieving nucleosome resolution with a fraction of the sequencing. By reaching the local equivalent of ˜317 billion unique contacts genome-wide, patterns of previously unseen, fine-scale, focal, and highly nested 3D interactions in gene-dense loci referred to herein as microcompartments, were discovered. Furthermore, and more generally, RCMC is much more sensitive to all known 3D interactions and reveals previously unresolvable looping interactions, including enhancer-promoter interactions.

The Examples demonstrate the superiority of RCMC when compared to prior methods such as Hi-C³¹, Micro-C¹², Micro-Capture-C (MCC)¹⁶, and Tiled-Micro-Capture-C (TMCC)¹⁷. Therefore, RMCC is superior to and can be distinguished from previously disclosed methods such as the method disclosed in U.S. Pat. No. 10,287,621, which is based on Hi-C (which uses restriction enzymes to cut DNA) as opposed to Micro-C (which uses MNase to generate mainly mono-nucleosomal fragments) and which is not a region capture protocol (it is promoter capture protocol in that the capture probes target all promoters in the genome).

The Examples demonstrates that for mapping genomic interactions within specific regions, RCMC outperforms genome-wide Hi-C³¹ (and U.S. Pat. No. 10,287,621) and Micro-C¹² at a fraction of the cost.

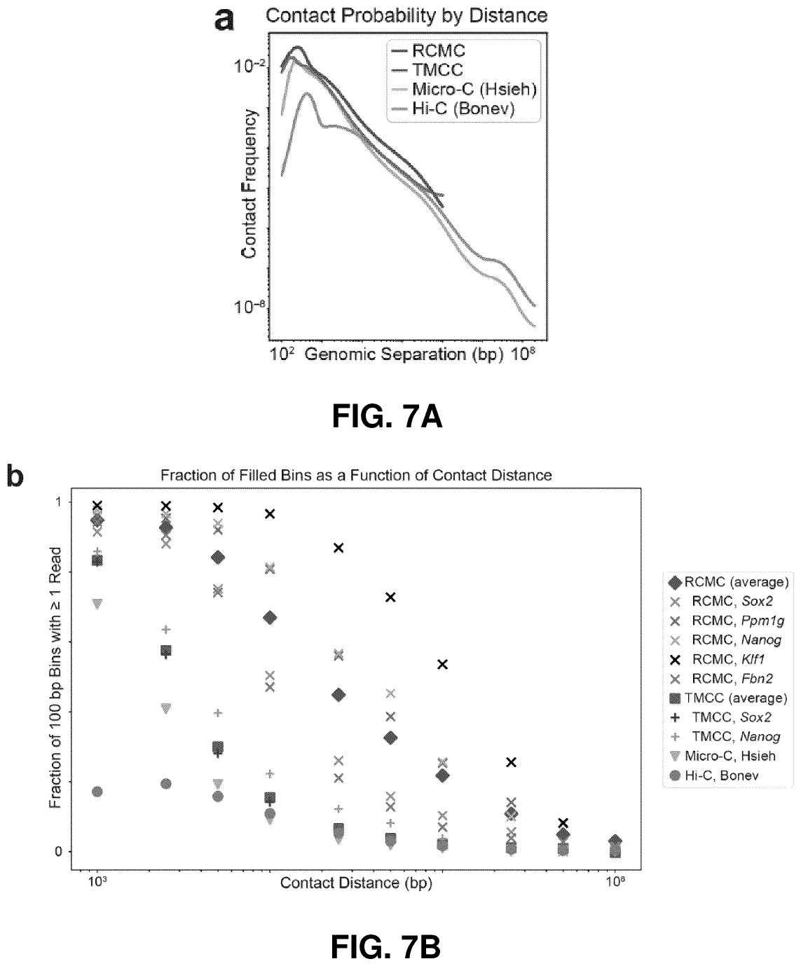

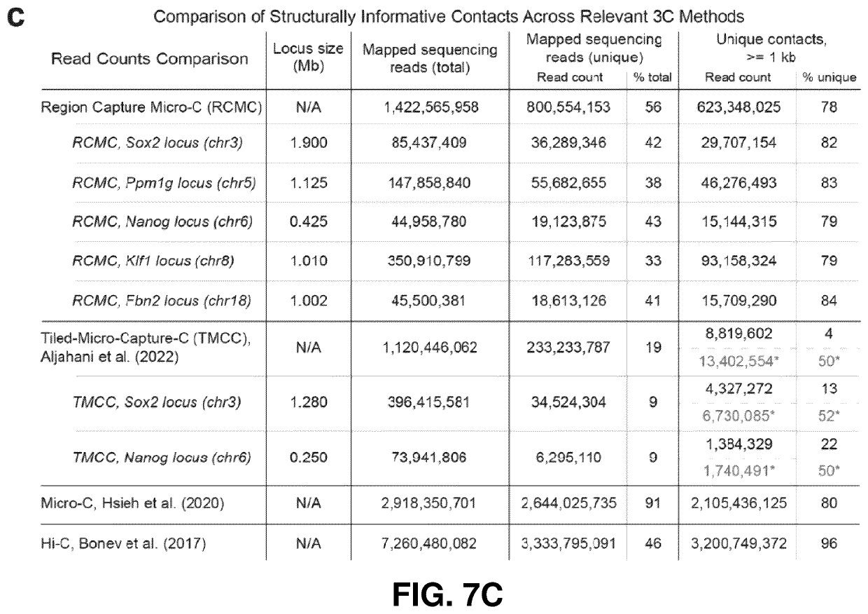

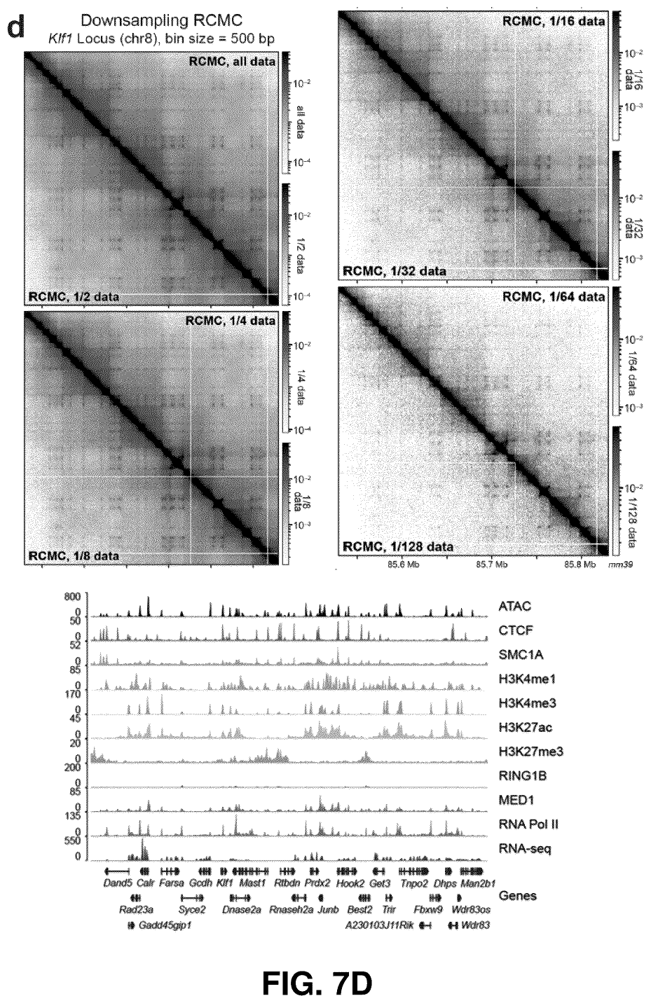

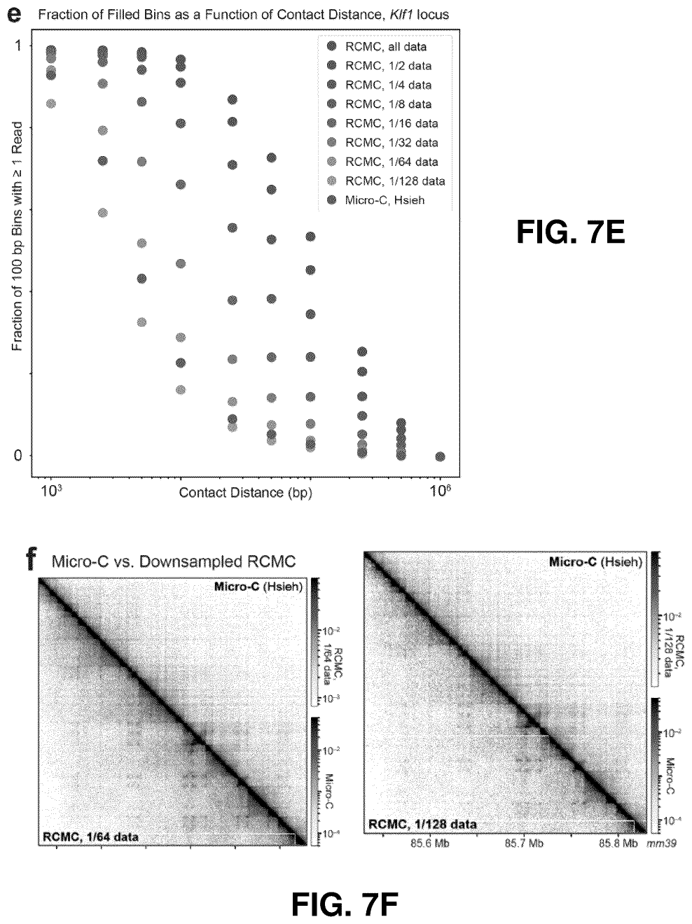

Compared to TMCC, RCMC is significantly superior. Even with similar total sequencing reads, RCMC captured ˜134 million unique ≥1 kb cis contacts in the target regions compared to just ˜9-13 million for TMCC, underscoring the more than one order of magnitude higher efficiency of RCMC. TMCC maps were noisier than RCMC. RCMC is ˜55-fold more efficient in capturing unique and structurally informative interactions than TMCC (FIG. 7C; RCMC efficiency=623,348,025/1,422,565,958=43.8% of all reads; TMCC efficiency=8,819,602/1,120,446,062=0.787% of all reads).

### I. Definitions

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

A binding pair” refer to at least two moieties (i.e., a first half and a second half) that specifically recognize each other in order to form an attachment. Suitable binding pairs include, for example, biotin and avidin or biotin and derivatives of avidin such as streptavidin and neutravidin.

The term “chromosome” as used herein refers to naturally occurring nucleic acid sequence.

The term “crosslink”, “crosslinking”, or “cross-link” is intended to mean stable chemical association between two compounds, such that they may be further processed as a unit. Such stability may be based upon covalent and/or non-covalent bonding. For example, nucleic acids and/or proteins may be crosslinked by chemical agents (i.e., for example, a fixative) such that they maintain their spatial relationships during routine laboratory procedures (i.e., for example, extracting, washing, centrifugation, etc.). Many chemicals are capable of providing crosslinking, including but not limited to, disuccinimidyl glutarate, formaldehyde, dimethyl adipimidate (DMA), glutaraldehyde, and ethylene glycol bis(succinimidyl succinate).

References to the term “fragments” as used herein, refers to any nucleic acid sequence that is shorter than the sequence from which it is derived. Fragments can be of any size, ranging from several megabases and/or kilobases to only a few nucleotides long. Fragments are suitably greater than 5 nucleotide bases in length, for example 10, 15, 20, 25, 30, 40, 50, 100, 250, 500, 750, 1000, 2000, 5000 or 10000 nucleotide bases in length. Fragments may be even longer, for example 1, 5, 10, 20, 25, 50, 75, 100, 200, 300, 400 or 500 nucleotide kilobases in length.

The term “first and second set of regions” are intended to mean nucleotides sequences that are located at different positions within the genome but that under specific conditions comes into contact with each other and by that are able to cooperate and direct events that occurs within the cell such as expression or silencing of specific genes.

The term “fragmenting” as used herein is intended to mean a method by which a nucleotide sequence is fragmented/separated into smaller unit fragments.

“Labelling” or “labelled” refer to the process of distinguishing a target by attaching a marker, wherein the marker includes a specific moiety having a unique affinity for a ligand where the marker and ligand are a binding pair.

The term “ligated or ligation” is intended to mean linkage of two nucleic acid sequences usually comprising a phosphodiester bond. The linkage is normally facilitated by the presence of a catalytic enzyme (i.e., a ligase) in the presence of co-factor reagents and an energy source (i.e., adenosine triphosphate (ATP)).

The term “labelled capture probe” is intended to mean a short sequence of nucleotides comprising a label that is capable of hybridizing to another nucleotide sequence. For example, the label may serve to selectively purify specific nucleic acid sequences of interest. Such a label may include, but is not limited to, biotin.

The term “regulatory element” is intended to mean a nucleic acid sequence that affects the expression of another genomic sequence. Examples are enhancers, repressors, insulators, silencers, and locus control regions.

### II. Region Capture Micro-C Method

RCMC is a chromosome conformation capture assay that combines improvements in Micro-C with tiled Capture of regions of interest, allowing deep mapping of 3D genome structure with relatively shallow sequencing.

Genomes are complex and are composed of nucleic acids and proteins as well as some other biological components. The activity of genes is tightly regulated to achieve biological functions at the right time and place. Each gene carries a region called the promoter, which is a short DNA sequence responsible for interpreting the signals in the cellular environment to decide whether the gene should be activated or not, and to which extent it should be activated. Specific proteins (transcription factors) bind to the promoter sequence to initiate assembly or disassembly of the protein machinery to either activate or inactivate its gene. Both secondary as well as the tertiary conformational structures of the genomes as well as the regulatory elements constitute the architecture that initiates and directs the events that occurs within a cell. Often there are DNA regions located distally in the genome fold onto the promoter sequences.

The three-dimensional conformation of chromosomes may be involved in compartmentalizing the nucleus and bringing widely separated functional elements into close spatial proximity. Understanding how chromosomes fold can provide insight into the complex relationships between chromatin structure, gene activity, and the functional state of the cell. Regions separated by many megabases can be immediately adjacent in 3-dimensional space. From the standpoint of regulation, understanding long-range interactions between genomic loci may be useful. For example, gene enhancers, silencers, and insulator elements might possibly function across vast genomic distances.

Distal DNA sequences (called enhancers) can bind to specific proteins. The interactions between enhancer-bound and promoter-bound proteins contribute to the decision whether the gene will be activated or not. This process is called distal regulation of genes. Promoters of genes are always found proximal to the genes; however, distal regulatory regions can be far away in the primary sequence of the genome and it is not possible to know which distal regulatory elements fold and act on to which promoter from the primary DNA sequence itself.

The disclosed methods allow a more in-depth resolution/interrogation of interactions between enhancers and promoters, making it possible to know which regulatory elements regulate which promoter.

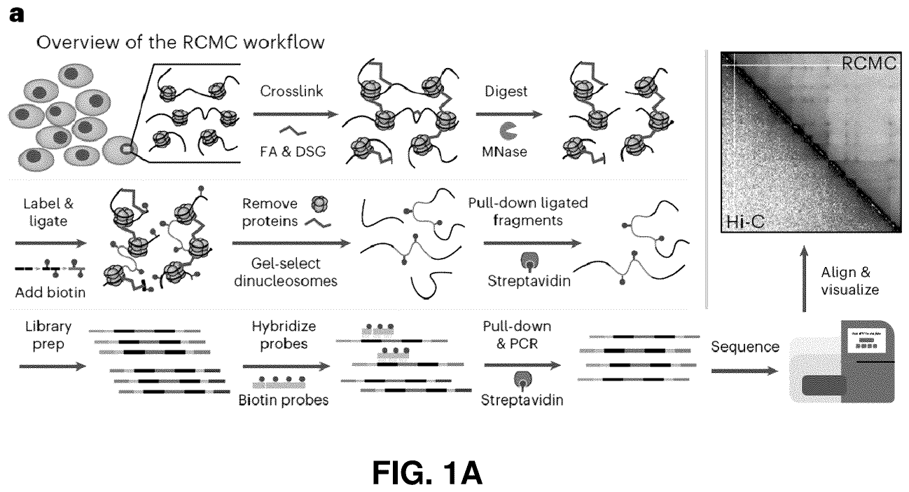

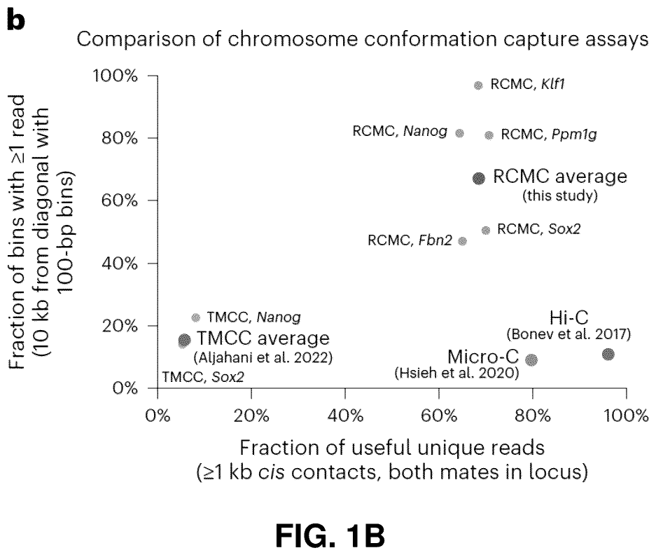

FIG. 1A details the RCMC workflow. In sum, cells are chemically fixed, nuclei are digested, preferably with MNase, and fragments are biotinylated, proximity-ligated, followed by decrosslinking, then the fragments are size-separated, gel-extracted, purified, and library-prepped. This ends the improved Micro-C protocol. The Micro-C protocol is followed by Region Capture. During Region Capture, biotin labelled probes designed against a region of interest are hybridized to the genome-wide Micro-C library, pulled down, PCR-amplified, and sequenced. After sequencing, mapping, and normalization, the data are visualized as a contact matrix. Thus, the improved Micro-C protocol results in a genome-wide library which is then subjected to region capture, following which, a RCMC sequencing library is obtained.

Accordingly, an RCMC method is provided for identifying nucleic acid segments which interact with a sub-group of target nucleic acid segments, the method including the steps of: (1) crosslinking genomic DNA from cells; (2) fragmenting the crosslinked genomic DNA to obtain fragments; (3) repairing the fragment ends, followed by fragment end labelling; (4) ligating nucleic acid fragments; (4) obtaining a purified and size-selected DNA fraction; (5) preparing a sequencing library from the purified ligated fragments; and (6) performing tiling region capture of a region of interest.

The cell sample size introduced into the crosslinking step should generate sample with a sufficient range of fragments to cover the genomic region, for example, more than 5 million (5M) Cells. In some forms, at least more than 5 million (5M) cells are introduced into the crosslinking step, for example, 6M, 7M, 8M, 9M, 11M, 12M, 13M, 14M, 15M, 16M, 17M, 18M, 19M, 20M, 21M, 22M, 23M, 24M, 25M, 26M, 27M, 28M, 29M, 30M. In preferred forms, at least 10M, at least 15M, at least, 20M, or at least 25M and up to 100 M cells are introduced into the crosslinking step. One can readily determine an upper limit of cells that allow for further processing.

In some forms, the chromatin in cells is crosslinked using a suitable crosslinking agent, preferably with at least two crosslinking agents and preferably in a single step including sequential crosslinking (i.e., double crosslinking), prior to quenching of the crosslinking reaction. Preferred crosslinking agents include disuccinimidyl glutarate (DSG) and formaldehyde. There can be cell losses throughout the crosslinking protocol e.g., if the protocol is started with 5M cells, by the time of freezing the cells, there may only be 3M cells remaining by the point where cells are flash-frozen; thus a 40% loss. Therefore, it is important to quantify the number of remaining cells after the crosslinking step to consistently bring it 1M or 5M cells to subsequent steps. Thus, in some forms, cells are counted following crosslinking to quantify losses during cell crosslinking and collection steps. This ensures that the ratio of reagents (e.g., MNase) to cell number is consistent during subsequent steps. Thus, this step helps ensure that the correct amount of cells are entering the protocol, and therefore ensures that the protocol provides the improved results seen herein.

Generally, higher numbers of cells increase the quality of the data output. As demonstrated in the Examples, about 25M cells were used for the full protocol; this is split into five tubes, each containing about 5M cells and taken through the protocol. Ligated fragments from these five samples were pooled to generate libraries. The protocol used about five of these ˜5M cell lots as an effective sample size to generate sample with a sufficient range of fragments to cover the genomic region. In some forms, the improved Micro-C protocol does not employ <5M cells or ˜5M cells (which may not generate sample with a sufficient range of fragments to cover the genomic region).

In some forms fragmenting the crosslinked genomic DNA results in nucleosome-sized fragments (150-200 bp) fragments and the method is performed using any suitable nuclease, more preferably using micrococcal nuclease (MNase), by contacting a cell composition with a MNase composition in an amount and a time effective for digestion of the chromatin. If the cells are from a frozen sample, the cells are thawed and resuspended in a buffer, in some forms containing bovine serum albumin (BSA; at about 100 μg/mL), for improved pelletization. In some forms, fragmenting the crosslinked genomic DNA does not include sonication.

In some forms, NaCl (about 200 mM) is added to reverse crosslinking reaction mix to improve reverse crosslinking efficiency and increase DNA yield.

The nucleosome-sized fragments are end labelled following fragment end repair, using as a label an agent that has a binding partner (of a binding pair) to which it binds by affinity binding. An example of a labelling agent is biotin.

In some forms, centrifugation speeds of 10,000×g in steps prior to proximity ligation are reduced to about 1,750×g, for example, when suspending and selecting cells prior to ligating nucleic acid fragments to reduce damage to cells due to high forces and increase the likelihood of capturing real interactions.

The ligation step ensures that free DNA ends can be ligated to each other, such that a first DNA fragment and a second DNA fragment are ligated to each other. This is the step where the folding of the genome is captured: since the three-dimensional structure of the genome is preserved by crosslinking, regions that were close to each other (i.e., interacting) at the time of crosslinking can be ligated to each other even though they are actually far away in the primary sequence of DNA. Then the crosslinking is reversed, and DNA is extracted. The material is now composed of DNA that contains sequences that were near each other in the three-dimensional space.

In some forms, obtaining a purified DNA fraction in the size range of ˜200-350 bp which contains predominantly ligated di-nucleosomes as well as other ligation products including footprints from a reverse-crosslinked sample, is done by isolating ligation products in the size range of ˜200-350 bp containing predominantly ligated di-nucleosomes using a suitable method, for example using gel electrophoresis (subjecting the sample to gel electrophoresis (for example, using 1% agarose gel) and extracting DNA in the size range of ˜200-400 bp from the 1% agarose gel). In some forms, ligated DNA contact fragments are isolated by pulling down label-bound fragments using a binding partner for the label. For example, where the label is biotin, streptavidin can be used to pull down biotin-bound fragments. When the binding partner is used in a form bound to beads, the beads are preferably used in a proportion of about 25-30 μl of beads per 5×10⁶ cells. The ligated fragments include at least a first and a second DNA region ligated to each other.

In some forms, the stage of library preparation includes one or more steps of: (i) end polishing, (ii) streptavidin purification, (iii) end repair & A-tailing, (iv) adapter ligation, (vi) bead washing, (vii) test PCR run, (ix) pool PCR run, (x) sample purification, (xi) sample quantification, and (xii) pooling of barcoded samples.

The disclosed methods provide improved evaluation and detection of direct intra- and inter-chromosomal interactions between remote regulatory elements; this information can be used to diagnose specific medical and/or biological conditions.

The RCMC protocol contains 3 sub-protocols: (A) RCMC Protocol Pre-Capture; (B) MNase Titration Protocol; and (C) Capture Protocol which are outlined in detail below as exemplary steps. It is within the abilities of one of ordinary skill in the art to vary the specific concentrations of reagents or types of reagents outlined below to obtain the same result depending on other experimental considerations such as sample size. Thus, the disclosed methods are not limited to the specific reagent/reagent concentration. Kits that perform similar functions to the specifically selected kits are commercially available and can be used interchangeably to the commercial kits and reagents disclosed in the detailed protocols below.

### A. RCMC Protocol, Improved Micro-C

This is the bulk of the RCMC workflow and covers all experimental steps from cell culture through Micro-C library prep.

In some forms, the RCMC method includes one or more stages of (1)-(8). In preferred forms, the RCMC method includes the following steps: 1) Prepare crosslinked chromatin from a sample of cells and count the cells following crosslinking; 2) Perform MNase titration; 3) Digest crosslinked chromatin; 4) Fragment end repair and labelling; 5) Proximity ligation and removal of unligated ends; 6) DNA purification and size selection; 7) Library preparation; and 8) Deep sequencing.

Step I. Prepare Crosslinked Chromatin from Cells

In some forms, cells are harvested, and their chromatins are crosslinked, optionally with one or more washing steps. The cells can be obtained from a variety of sources e.g., the cells can be obtained via cell culture or they can be from other sources such as primary cells or blood cells. Generally, higher numbers of cells increase the quality of the data output. In an exemplary form, about 25M cells are used for the full protocol; this is split into five tubes, each containing about 5M cells and taken through the protocol. In some forms, the ligated fragments from these five samples are pooled to generate libraries. In some forms, using five of these ˜5M cell lots is necessary to generate sample with a sufficient range of fragments to deeply cover the genomic region. In some forms, using only ˜ 1M or ˜5M cells is not sufficient to generate sample with a sufficient range of fragments to deeply cover the genomic region.

In some forms, the crosslinking is carried out using formaldehyde and disuccinimidyl glutarate (DSG) sequentially or simultaneously. In preferred forms, the crosslinking is carried out in a single step with formaldehyde and DSG simultaneously without any washing step(s) in between.

In an exemplary form, the stage of preparing crosslinked chromatin from cell culture involves the following steps. Culture cells in the appropriate conditions and medium. Harvest cells. In some forms, the harvesting step involves trypsinization of the cells, optionally one or more wash steps (e.g., with PBS) before and/or after trypsinization. In preferred forms, cell numbers are estimated after both at the beginning of cell harvesting, for example, after inactivating the trypsin with media, as well as at the end, just before freezing down cells. In one form, cells are pelleted by centrifugation for 5 min at 800×g at room temperature. In a preferred form, a PBS wash of the cells post-centrifugation is performed to ensure that all possible media and trypsin have been removed before proceeding to crosslinking. Remove disuccinimidyl glutarate (DSG) from the 4° C. fridge during these steps, allowing it to equilibrate to room temp. Once you know the number of cells you will be harvesting (post-counting), you can simultaneously begin preparing the crosslinking solution for step 3.

In some forms, simultaneous crosslinking with DSG and formaldehyde is performed. In some forms, crosslinking with DSG and formaldehyde is performed separately, i.e., treatment of a sample with a first crosslinking agent (DSG/formaldehyde), followed by a quenching step, followed by treatment with the second crosslinking agent (DSG/formaldehyde). In preferred forms, simultaneous crosslinking with DSG and formaldehyde is performed i.e., without a quenching step between treatment with DSG and formaldehyde.

### (a) Simultaneous Crosslinking

For simultaneous crosslinking, the following steps are preferably followed.

### Crosslinking Using DSG

In preferred forms, a DSG³ (long crosslinker, 7.7 Å) 300 mM stock solution (100×) in DMSO (MW=326.26; 20 mg in 200 μL DMSO) is freshly prepared. Dilute 100 times with room-temperature PBS (200 μL to 19.8 mL) to provide a working solution of 3 mM of DSG. Concentrations of DSG from about 1 to about 5 mM can be used, for example, 1, 2, 3, 4 and about 5 mM.

In some forms, resuspend a cell pellet in the long crosslinker solution at a concentration of 1×10⁶ cells/mL. Incubate for about 35 minutes at room temperature with mixing. Resuspend the pellet first with a 1-mL low-retention tip and then add the rest of the media with a larger pipette without touching the cells. Make sure that mixing is done gently-harsh nutation can cause cell damage and may result in significant cell loss.

### Crosslinking Using Formaldehyde

In some forms, about 16% formaldehyde is added (dropwise, but still rapidly) to a final concentration of about 1% to the DSG-containing crosslinking cell mixture (e.g., 2 mL for a 30 mL samples) and allow crosslinking to continue incubating for 10 min at room temperature with gentle mixing. Preferably, add the formaldehyde in the fume hood; if there are several samples, staggering crosslinking is preferred. Thus, about 10-about 20% formaldehyde can be added, for example, 10, 11, 12, 13, 14, 15, 16, 17, 18% and up to 20% although about 16%, is preferred.

### Quench Crosslinking Reaction

Buffer, preferably, Tris is added to the reaction to quench the reaction. In some forms, Tris pH 7.5 is added dropwise to a final concentration of 0.375 M to quench the reaction (e.g., for a 30 mL sample, 18 mL using 1 M Tris or 6.9 mL using 2 M Tris). Incubate for 5 min at room temperature. Centrifuge for 5 min at 850×g at 4° C. Aspirate supernatant.

### (b) Separate Crosslinking

For separately crosslinking with DSG and formaldehyde, the following steps are followed.

### Crosslinking Using Formaldehyde

Preferably, prepare enough base media (without FBS) or PBS to resuspend cells at a concentration of about 1×10⁶ cells/mL (max. 33 mL in 50 mL tube for adequate mixing) and to it is added formaldehyde, for example, about 16% formaldehyde to a final concentration of 1% (e.g., 2 mL for 30 mL sample) in the fume hood to make the formaldehyde fixation media. Preferably, add the formaldehyde in the fume hood.

Resuspend cells in the formaldehyde fixation media. Incubate for 10 min at room temperature with mixing. Resuspend the pellet first with a 1-mL low-retention tip and then add the rest of the media with a larger pipette without touching the cells. Make sure that mixing is done gently-harsh nutation can cause cell damage and may result in significant cell loss.

To quench the reaction, add Tris pH 7.5 dropwise to a final concentration of 0.375 M (e.g., for a 30 mL sample, 18 mL using 1 M Tris or 6.9 mL using 2 M Tris). Incubate for 5 min at room temperature. Centrifuge for 5 min at 850×g at 4° C. Aspirate supernatant.

Wash cells twice with 1× cold PBS at a concentration of 1×10⁶ cells/mL. Each time, centrifuge for 5 min at 850×g at 4° C. and aspirate supernatant.

### Crosslinking Using DSG

Freshly prepare a DSG³ (long crosslinker, 7.7 Å) 300-mM stock solution (100×) in DMSO (MW×326.26; 20 mg in 200 μL DMSO) to provide a long crosslinker solution. Dilute 100 times with room-temperature PBS (200 μL to 19.8 mL).

Resuspend cell pellet in the long crosslinker solution at a concentration of 1×10⁶ cells/mL. Incubate for 45 minutes at room temperature with mixing. Resuspend the pellet first with a 1-mL low-retention tip and then add the rest of the media with a larger pipette without touching the cells. Make sure that mixing is done gently-harsh nutation can damage cells and may result in significant cell loss.

To quench the reaction, add Tris pH 7.5 dropwise to a final concentration of 0.375 M (e.g., for a 30 mL sample, 18 mL using 1 M Tris or 6.9 mL using 2 M Tris). Incubate for 5 min at room temperature. Centrifuge for 5 min at 850×g at 4° C. Aspirate supernatant.

Regardless of whether crosslinking was done simultaneously or separately, proceed as outlined below.

### (c) Cell Counting

Wash cells. In some forms, cells are washed with 1× cold PBS at a concentration of 1×10⁶ cells/mL followed by centrifugation for 5 min at 850×g at 4° C. Aspirate supernatant. Resuspend cell pellet to a desired volume of cold PBS to aliquot the sample into multiple tubes (e.g., 30×10⁶ cells resuspended to 6 mL and aliquoted to 6 tubes, 1 mL each, will yield 5×10⁶ cells/tube).

Re-count cells to gauge loss during crosslinking and determine the total number of feasible aliquots. Re-counting here is critical for establishing consistency in how many cells actually enter the rest of the protocol. Cell losses of typically about 20% to about 45% can occur as a result of the crosslinking process.

Aliquot the sample and store. Centrifuge for 5 min at 850×g at 4° C. Aspirate supernatant. Snap-freeze cell pellets in liquid nitrogen. Store at −80° C. until needed (up to several years).

When conducting Micro-C instead of RCMC, the total cellular input for the next stage is about 1×10⁶-10×10⁶. When conducting RCMC, in some forms, a total cellular input of about >5×10⁶-20×10⁶ cells is taken to the next stage, spread across tubes each containing about 5×10⁶ cells. In preferred forms, a total input of about 25×10⁶ cells is taken forward for RCMC, spread across five sets of about 5×10⁶ cells. Higher cellular inputs to subsequent stages buffer against potential sample loss throughout the protocol steps and minimize the likelihood of insufficient library complexity for target enrichment.

### (d) MNase Titration

Prior to the Step II, MNase titration is necessary. The RCMC workflow relies on digesting chromatin to primarily mononucleosomally-sized (e.g., 150-200 bp) fragments. Over-digestion of chromatin leads to short fragments and poor proximity ligation, whereas under-digestion risks mapping fewer contacts than possible due to the lower number of mono-nucleosomes available for ligation.

Digestion time and conditions may vary from cell-type to cell-type and can vary by fixation. In preferred forms, an MNase titration is carried out to determine the ideal MNase digestion amounts and conditions before proceeding with the rest of the protocol.

In preferred forms, a new MNase titration is conducted for each new batch of crosslinked cells.

In some forms, the MNase Titration Protocol includes one or more stages of (I)-(III). In preferred forms, the MNase Titration Protocol includes stages of (I)-(III).
(I) Prepare crosslinked chromatin from cells,
(II) Digest crosslinked chromatin with Micrococcal Nuclease (MNase), and
(III) Isolate DNA and visualize the titration.

Further details on the each of Stages (I)-(III) above are provided in Sub-protocol (B) MNase titration Protocol. Before chromatin digestion, it is necessary to perform the MNase titration to determine the optimal concentration of MNase.

### Step II. Digest Crosslinked Chromatin

In some forms, one of more of the following steps are performed to digest crosslinked chromatin with MNase.

Cell membranes were solubilized to extract intact nuclei by resuspending crosslinked 5 M cell pellets in Micro-C Buffer #1 (MB #1). An exemplary MB #1 is provided in Table 1 below.

TABLE 1
“complete” MB#1 solution (Prepared fresh)

| Stock | 5 mL | 10 mL | Final |
|---|---|---|---|
| MB#1 | 4850 μL | 9700 μL | 50 mM NaCl, 10 mM Tris, 5 |
| 10% NP-40 | 100 μL | 200 μL | mM MgCl₂, 1 mM CaCl₂, 0.2% |
| 100 × PIC | 50 μL | 100 μL | NP-40 alternative, 1X PIC. |
| | | | Use protein-grade NP-40 \| make |
| | | | sure to use NP-40 Alternative, not |
| | | | NP-40. |
| | | | PIC = Protease inhibitor cocktail |
| | | | dissolved in MB#1, aliquoted, and |
| | | | stored in −20° C. |

A preferred procedure is outlined in detail below.

Thaw cell pellets of the desired size, preferably totaling about 25×10⁶ cells per sample. In some forms, cell pellets containing about 1 or 5×10⁶ cells are placed on ice and resuspended in complete MB #1 to a concentration of about 1×10⁶ cells/100 μL. In preferred forms, a total of 25×10⁶ cells per sample can be used. Incubate for 20 min on ice. Centrifuge at ˜1,750×g for 5 min at 4° C. Discard supernatant, leaving ˜10-20 μL behind. Supernatant removal should be done with a P200 pipet tip; for larger volumes, append the tip to a P1000 tip. Spins can be performed at <3,000×g until DNA extraction as long as a pellet clearly forms with little nuclear loss in the suspension. In preferred forms, centrifugation at ≤2,000×g should be sufficient until at least proximity ligation. Centrifugal forces ≤2000×g are preferably to reduce the damage to the cells.

Wash nuclei pellet in complete MB #1. In preferred forms, the nuclei pellet is washed in complete MB #1 at a concentration of 1×10⁶ cells/100 μL, resuspending the pellet up & down with a pipette. Centrifuge at ˜1,750×g for 5 min at 4° C. Discard supernatant with a P200 pipette tip, removing as much liquid as possible without disturbing the pellet. Thaw MNase; partially used aliquots are stored at −20° C. (use no more than 3 times), fresh aliquots are stored at −80° C.

Resuspend nuclei pellet in complete MB #1, preferably at a concentration of 1×10⁶ cells/100 μL, pipetting up & down.

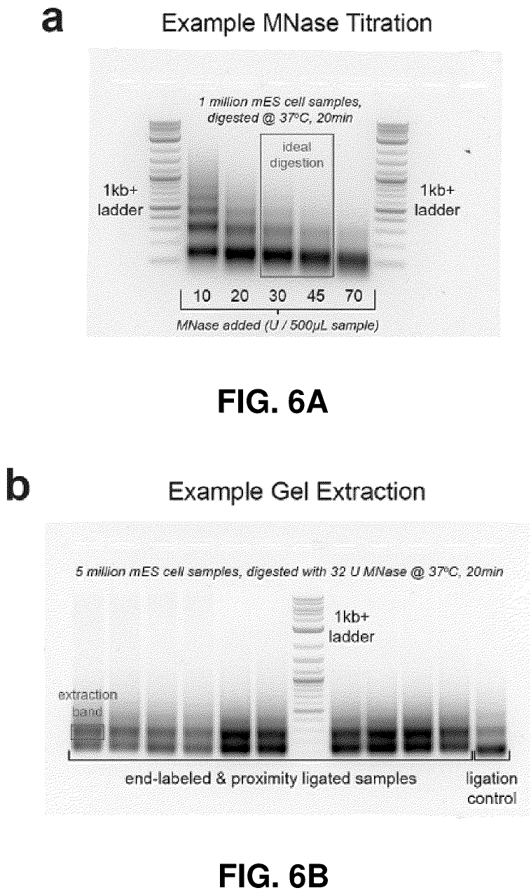

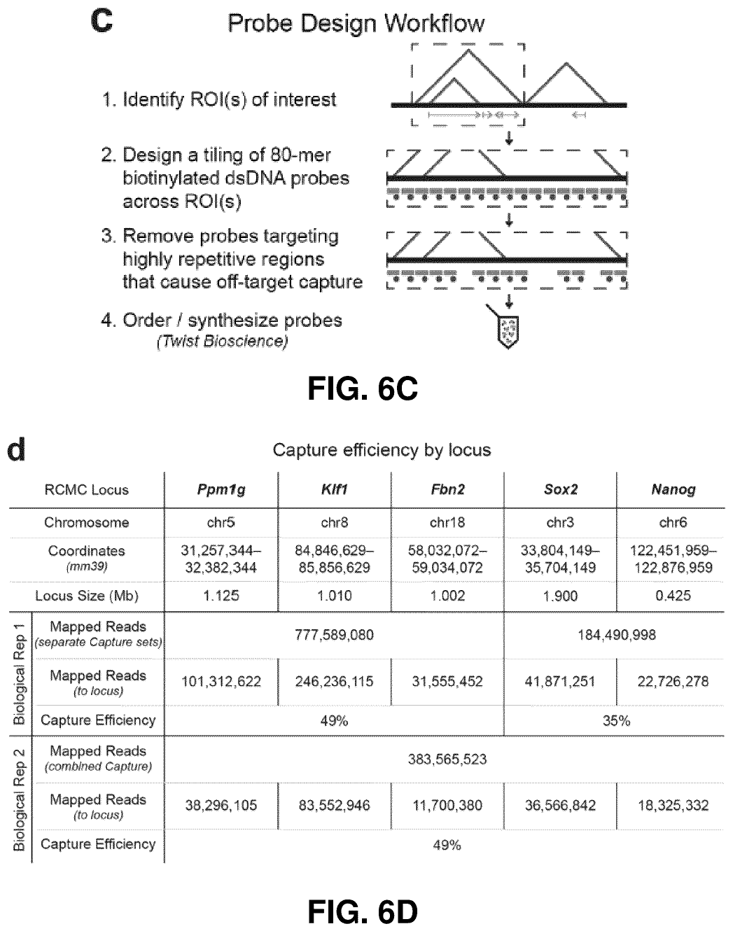

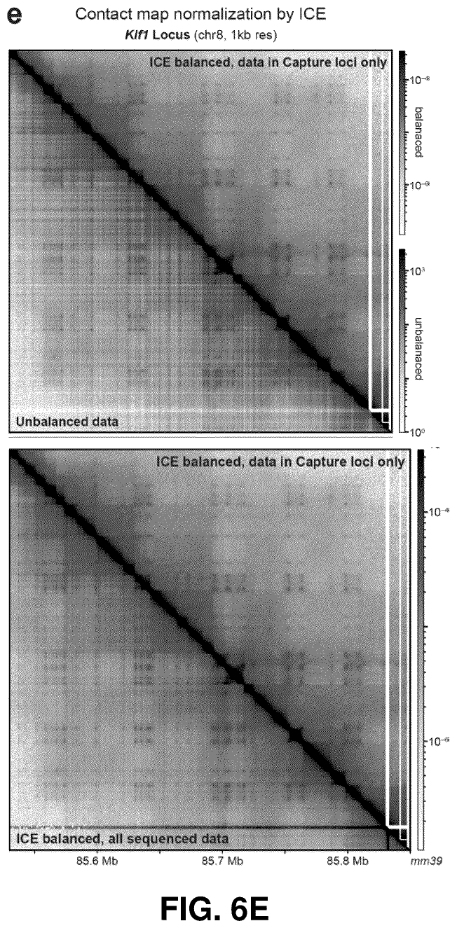

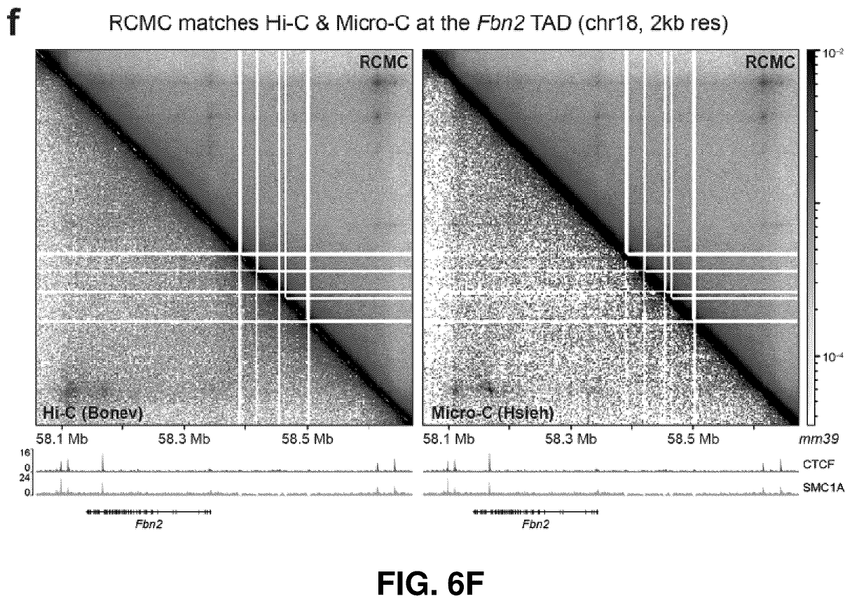

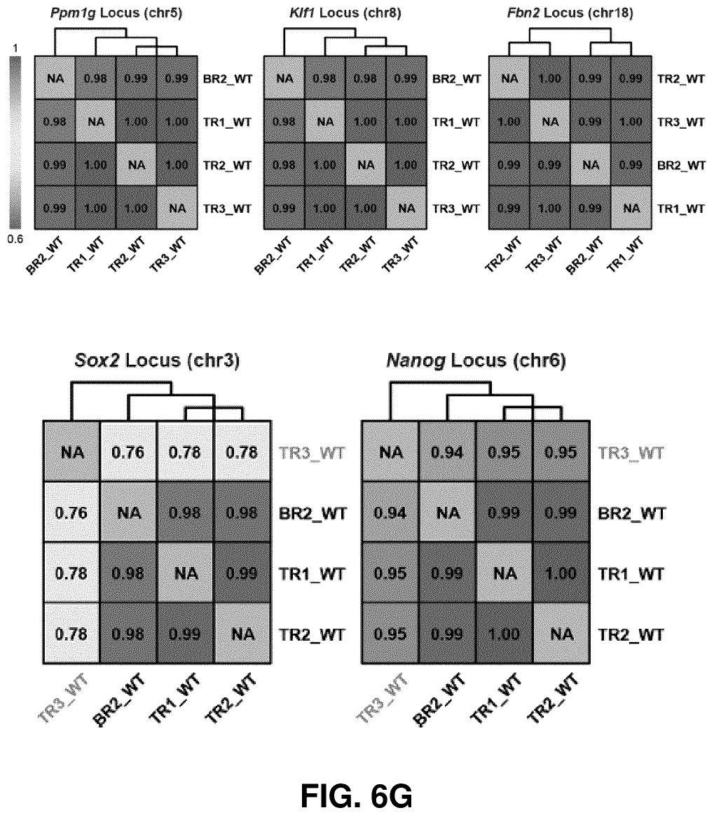

Add the appropriate amount of MNase (as determined by an MNase titration experiment on the same sample batch) to digest chromatin, preferably to an 80-90% monomer/20-10% dimer ratio with a low (<5%) yet detectable trimer concentration. Vortex briefly and incubate using the same conditions used in the MNase titration, usually 10-20 min at 37° C. with shaking at 850-1,000 RPM using a thermomixer; 20 min of digestion with a lower concentration of MNase is preferable over 10 min of digestion with a higher concentration of MNase. The digestion step is critical to ensure efficient ligation. Over-digestion removes DNA ends needed for ligation, whereas under-digestion fails to generate enough monomers to achieve high ligation efficiency. For example, FIG. 6A demonstrates how the ideal digestion condition can be determined in an MNase titration. As shown in lane 10 of FIG. 6A, underdigestion led to insufficient mono-nucleosomes for ligation. Also, as shown in lane 70 of FIG. 6A, overdigestion led to the reaction containing exclusively mono-nucleosomes, and the linker DNA becoming overly digested, thereby resulting in inefficient ligation.

Transfer the nuclei back onto ice and promptly stop the reaction, for example, by adding 500 mM EGTA to a final concentration of 4 mM (0.8 μL for a 100 μL reaction). Vortex briefly to mix. Incubate for 10 min at 65° C. to ensure complete inactivation of the MNase. Centrifuge at 1,750-2,000×g for 5 min at 4° C. Discard supernatant with a P200 tip. EGTA is a stronger Ca²⁺ chelator than EDTA. EGTA efficiently chelates Ca²⁺ ions needed by the MNase and quenches the reaction. In preferred forms, EGTA is used as the Ca²⁺ chelator. Alternatively, a Ca²⁺ chelator having chelating efficiencies similar to EGTA can be used.

Wash/rinse nuclei pellet in a cold buffer, for example MB #2 (Table 2), for example, 1 mL of cold MB #2 twice. Centrifuge for 5 min at 1,750-2,000×g at 4° C. Discard supernatant with a P200 pipette tip. After the second wash, try and remove as much liquid as possible without disturbing the pellet.

TABLE 2
MB#2 solution

| Stock | Final | For 500 mL |
|---|---|---|
| 5M NaCl | 50 mM | 5 mL |
| 1M Tris-HCl pH 7.5 | 10 mM | 5 mL |
| 1M MgCl₂ | 10 mM | 5 mL |

Bring volume to 500 mL with ddH₂O and sterile filter. In some forms, BSA is added to a final concentration of 100 μg/mL to aid pelletization.

In some forms, centrifugation at this stage (e.g., in steps 1, 2,5, and 6) is performed at less than 10,000×g, less than 9,000×g, less than 8,000×g, less than 7,000×g, less than 6,000×g, less than 5,000×g, less than 4,000×g, less than 3,000×g, less than 2,000×g. In preferred forms, centrifugation at this stage is performed at less than 2,000×g. In one form, centrifugation at this stage is performed at less than 1,750×g.

### Step III. End Repair and Labelling

In some forms, the stage of repairing fragment ends involves a step of end chewing followed by a step of end labelling.

To generate blunt ends on digested DNA fragments before proximity ligation and add biotinylated nucleotides, a series of enzymatic processing steps are performed.

### End Chewing

First, to catalyze the addition of 5′-phosphate groups and the removal of 3′-phosphate groups, digested samples generated from 5 M cell inputs are incubated in an end-repair reaction.

An exemplary end-repair reaction is shown in Table 3.

TABLE 3
End-repair reaction

| | | | | | A pre-mix of these reagents |
|---|---|---|---|---|---|
| | 5 × 10⁶ | | 1 × 10⁶ | | can be made just |
| | cells | | cells | | before reaction addition. |
| Stock | 100 μL | | 50 μL | | Final |
| Chromatin | pellet | | Pellet | | 50 mM NaCl, 10 mM Tris-HCl |
| H₂O (first) | 50 | μL | 25 | μL | pH 7.5, 10 mM MgCl₂, 100 |
| 10 × NEBuffer 2.1 | 10 | μL | 5 | μL | μg/mL BSA; 2 mM ATP; |
| 10 mM ATP | 20 | μL | 10 | μL | 5 mM DTT (freshly |
| | | | | | prepare 0.01543 g |
| 100 mM DTT | 5 | μL | 2.5 | μL | DTT in 1 mL HEPES buffer) |
| 10 U/μL T4 PNK | 5 | μL | 2.5 | μL | ~25-50 U / 300 pmol of DNA. |
| (last) | | | | | |
| 5 U/μL Klenow | 10 | μL | 5 | μL | ~1 U / 1 μg of DNA |

Fragment (after
initial incubation)

Incubate for 15 minutes at 37° C., shaking at 1,000 RPM. T4 PNK catalyzes the transfer of a phosphate to the 5′ ends of DNA and removes phosphates from 3′ ends. 5′ fragment overhangs for end-blunting and labelling are created. Add 5 U/μL Klenow Fragment. Incubate for 15 minutes at 37° C., shaking at 1,000 RPM. In the absence of dNTPs, the Klenow fragment only possesses a 3′-5′ exonuclease activity, creating a single-stranded DNA that can be labelled with biotin in the next step.

Next, a mixture of dNTPs in end-labelling buffer is added to the reaction for end labelling as discussed further below. Exemplary end labelling components are shown in Table 4.

TABLE 4
End labelling reaction

| | | | | | A pre-mix of these |
|---|---|---|---|---|---|
| | | | | | reagents can be |
| | 5 × 10⁶ | | 1 × 10⁶ | | made just before |
| | cells | | cells | | reaction addition. |
| Stock | 150 μL | | 75 μL | | Final |
| Chromatin | 100 | μL | 50 | μL | 33 mM NaCl, 1.3 mM ATP |
| | | | | | (from previous step) |
| 1 mM Biotin- | 10 | μL | 5 | μL | dNTPs: 66 μM each |
| dATP | | | | | 23 mM Tris-HCl pH 7.5, |
| 1 mM Biotin- | 10 | μL | 5 | μL | 10 mM MgCl₂, |
| dCTP | | | | | 0.33 mM ATP, 6.67 mM |
| 10 mM dTTP | 1 | μL | 0.5 | μL | DTT |
| 10 mM dGTP | 1 | μL | 0.5 | μL | |
| 10X T4 DNA | 5 | μL | 2.5 | μL | |
| ligase buffer | | | | | |
| 20 mg/mL BSA | 0.25 | μL | 0.125 | μL | 100 μg/mL BSA |
| (200X) | | | | | |
| H₂O | 22.75 | μL | 11.375 | μL | |

Incubate the reaction mixture for at least 45 minutes at 25° C. with interval mixing at 1,000 RPM. Use a thermomixer that allows interval mixing: 1 minute of shaking, followed by 3 minutes still, repeated. Add 500 mM EDTA to a final concentration of 30 mM (9 μL in 150 μL) to quench the reaction. Briefly vortex and incubate at 65° C. for 20 minutes. Centrifuge at 1,750-2,000×g for 5 minutes at 4° C. Discard supernatant with P200 tips.

Rinse once with 1 mL of cold MB #3, pipetting up and down. An exemplary formulation for MB #3 is shown in Table 5.

TABLE 5
MB#3 solution

| Stock | Final | For 500 mL |
|---|---|---|
| 1M Tris-HCl pH 7.5 | 50 mM | 25 mL |
| 1M MgCl₂ | 10 mM | 5 mL |

Bring volume to 500 mL with ddH₂O and sterile filter. In some forms, BSA is added to a final concentration of 100 μg/mL to aid pelletization.

Centrifuge at 1,750-2,000×g for 5 minutes at 4° C. Discard supernatant with P200 tips.

### Step IV. Proximity Ligation and Removal of Unligated Ends

In some forms, the stage of proximity ligation and purge of unligated ends includes the steps of (1) proximity ligation, (2) removal of biotin-dNTPs from unligated ends, and (3) reverse crosslinking.

Proximity ligation is performed by incubating labelled chromatin in a ligation reaction, exemplified herein in Table 6.

TABLE 6
Proximity ligation reaction

| | | | | | A pre-mix of these |
|---|---|---|---|---|---|
| | | | | | reagents can be |
| | 5 × 10⁶ | | 1 × 10⁶ | | made just before |
| | cells | | cells | | reaction addition. |
| Stock | 500 μL | | 250 μL | | Final |
| Chromatin | pellet | | pellet | | 50 mM Tris, 10 mM MgCl₂, |
| H₂O | 422.5 | μL | 211.25 | μL | 1 mM ATP, |
| 10x T4 DNA ligase buffer | 50 | μL | 25 | μL | 10 mM DTT |
| 20 mg/mL BSA (200X) | 2.5 | μL | 1.25 | μL | 100 μg/mL BSA; |
| 400 U/μL T4 DNA ligase | 25 | μL | 12.5 | μL | Add T4 DNA ligase last |

Incubate for at least 2.5 hours (overnight is alright) at 25° C. with slow rotation using a gentle nutator.

The samples are centrifuged at 3,000×g for 5 minutes at 4° C. Aspirate with P200 tips, and the supernatant is discarded.

To remove biotinylated dNTPs from all unligated fragment ends, samples are digested by an exonuclease, for example, 1,000 U Exonuclease III. An exemplary unligated fragment removal reaction is shown in Table 7.

TABLE 7
Removal of biotin-dNTPs from unligated ends

| | | | A pre-mix of these |
|---|---|---|---|
| | | | reagents can be |
| | | | made just before |
| | 5 × 10⁶ cells | 1 × 10⁶ cells | reaction addition. |
| Stock | 200 μL | 100 μL | Final |
| Chromatin | pellet | pellet | 10 mM Bis-Tris-Propane-HCl, |
| H₂O | 170 μL | 85 μL | 10 mM MgCl₂, |
| 10X NEBuffer #1 | 20 μL | 10 μL | 1 mM DTT |
| 100 U/μL Exonuclease III | 10 μL | 5 μL | |

Incubate at 37° C. for 15 min with interval mixing (1 minute shaking @ 1,000 RPM, 3 minutes still).

The samples are then subjected to reverse crosslinking. To prepare ligated DNA for library generation, DNA is reverse crosslinked and proteins and RNA are digested in a reverse crosslinking reaction. An exemplary reverse crosslinking reaction is shown in Table 8.

TABLE 8
Reverse crosslinking reaction

| | 5 × 10⁶ cells | | 1 × 10⁶ cells | | | |
|---|---|---|---|---|---|---|
| Stock | 265 μL | | 133 μL | | Final | |
| Chromatin | 200 | μL | 100 | μL | | |
| 20 mg/mL | 26 | μL | 13 | μL | 2 mg/mL | |
| Proteinase K | | | | | Proteinase K | |
| 10% SDS solution | 26 | μL | 13 | μL | 1X SDS | |
| 5M NaCl | 10.4 | μL | 5.2 | μL | 250 | mM |
| 10 mg/mL RNaseA | 2.6 | μL | 1.3 | μL | 0.1 | mg/mL |

Incubate at 65° C. overnight while shaking at 1,000 RPM.

In some forms, the reaction step of reversing crosslinking includes NaCl, at a concentration of between about 10 mM and about 500 mM, preferably between about 100 mM and about 300 mM, more preferably, between 200 mM and about 250 mM, inclusive.

Preferably, a master mix is not prepared since the SDS would be extra concentrated, and/or filter tips are used once RNaseA has been added.

In some forms, the reaction mixture is incubated at 65° C. overnight. In some cases, if incubated in a water bath, the sample lids should be carefully covered with Parafilm to avoid any accidental lid openings. In exemplary forms, the reaction is incubated at 65° C. while shaking at 1,000 RPM in a thermomixer.

Following reverse crosslinking, the sample may be stored at 4° C. between any protocol steps to proceed at a later time point, e.g., the following day.

### Step V. DNA Purification and Size Selection

In some forms, the stage of DNA fragment purification includes one or more of the following steps: (a) optional DNA extraction & precipitation steps; (b) purification of reverse-crosslinked DNA; (c) size selection of 200-350 bp DNA; and (d) purification of gel-extracted DNA.

a. Optional Phenol-Chloroform: Isoamylic Alcohol (PCI) Extraction

In some forms, all of the following steps are performed in a fume hood to extract DNA. In preferred forms, this step of DNA purification by PCI extraction is skipped. In some forms, skipping this step can further increase yields without adversely affecting sample quality. An exemplary DNA extraction protocol is provided below.

Transfer sample to labelled phase-lock tubes. Phase-lock tubes are known in the art and are commercially available. An exemplary phase-lock tube that can be used is 5PRIME Phase Lock Gel Light tubes (Quantabio Cat #2302820). A quick spin may be necessary pre-transfer to remove gel from the tube lid. Add an equivalent volume of PCI to the sample volume (if using 5×10⁶ cells, add 265 μL of PCI to 265 μL of sample). PCI often comes with a top “containment” layer of liquid-avoid pipetting from this top layer and instead pipette from the PCI solution beneath it. Vortex for 20 seconds and then spin for 15 minutes at 19,800×g at room temperature. Transfer the upper layer to a new low-retention tube. Optionally, add 50 μL TE to each phase-lock tube and vortex again. Spin for 10 minutes at 19,800×g at room temp. This second spin will help with the removal of any residual aqueous layer DNA. Transfer the upper layer to the previously used low-retention tube.

### Optional Ethanol Precipitation

In some forms, DNA is precipitated using ethanol precipitation. In preferred forms, this step of DNA purification by ethanol precipitation is skipped. An exemplary DNA precipitation protocol is as follows:

Add 0.1×volumes of 3M sodium acetate and 2.5×volumes of cold 100% ethanol to the sample volume. Invert the tube and incubate for at least 1 hour at −80° C. (overnight is alright but not necessary). Spin at 19,800×g for 15 minutes at 4° C. Discard ethanol with a P200 pipette and wash the pellet with 1 mL of cold 80% ethanol. A pellet may be faint or impossible to see. This would likely result from not starting with many cells. Spin at 19,800×g for 5 minutes at 4° C. Remove as much ethanol as possible (if needed spin the tube briefly once again) and air dry the pellet for 4 minutes at 37° C. with an open lid. The pellet shouldn't have a discernable layer of ethanol on it, but it also shouldn't be super dry. Add 50 μL of TE buffer on top of the pellet and let it dissolve at 37° C. for 30 minutes. Intermittently vortex and tabletop spin down to help dissolve all DNA. Cast a gel (usually 100 mL of a 1% gel made using regular agarose with SYBR Safe stain in the 12-well turquoise cassettes) at this time so it can set as you use the DCC kit.

b. DNA Purification

In some forms, DNA purification is carried out using the Zymo DNA Clean & Concentrator (e.g., DCC-25) kits following manufacturer's instructions. However, any suitable DNA purification method and kit can be used.

In preferred forms, the following DNA purification steps are used following the manufacturer's instructions for the DCC-25 kit, with modifications.

Add 300 μL of Zymo DNA binding buffer per 50 μL of DNA sample and load to the column. Wash twice with 400 μL of wash buffer. Perform a quick “dry spin” after the second wash to remove any residual wash buffer. Elute twice, each time with 25 μL of EB (50 μL total). To maximize yield, warm up elution buffer to 60-70° C. before adding it to the middle of the column filter. Be mindful of the recommended elution volume for the column, and adjust/increase volumes accordingly to ensure saturation.

c. Size-Selection of Predominantly Di-Nucleosomal DNA in the Size Range of ˜200-350 bp

In some forms, the size selection of dinucleosomal DNA includes one or more of the following steps. In preferred embodiments, this step is carried out using 1% TAE agarose gel. In further preferred embodiments, 3.5% TAE or 3% TBE agarose or low melting point agarose gel is not used. However, any commonly used DNA gel mixture can work for this step.

Quantify the sample concentration, for example by using a Nanodrop or Qubit. This should help you benchmark extraction yield and can help avoid “overloading” a gel well. For example, loading 1-5 μg of DNA per well of a standard 10-well gel is recommended, but more than 7 μg may lead to gel running issues. In some forms, when there is more DNA, the DNA can be split among two or more lanes. In preferred forms, DNA from a single 5M sample tube is split across two adjacent gel wells and recombined following gel extraction.

Add 10× Orange G loading dye to the sample in greater than 10× conc. (e.g., add 12 μL dye to 50 μL sample). This is necessary to densify the sample, so it sits in the gel well and doesn't float out. Do not vortex samples to mix; this can trap air bubbles in the sample and cause it to float out of the well.

Load the sample(s) onto the gel; if necessary, load to two large wells. Also load 1 kb+ DNA ladder. Run the gel, for example, at 120V for 60 minutes.

Cut the band containing the ligation products. In one form, use a GelDoc Imager to image the gel and cut out the DNA band containing di-nucleosomal fragments and associated ligation products. Cut the band between 250-400 bp or 200-350 bp to excise the entirety of the band. Avoid cutting below 200 bp to eliminate monomers.

d. Extraction of Predominantly Di-Nucleosomal DNA

In some embodiments, DNA fragments (˜200-400 bp or ˜250-350 bp) containing predominantly di-nucleosomes are isolated by gel extraction for example, from a 1% agarose gel. In preferred embodiments, DNA fragments (˜200-350 bp) containing predominantly di-nucleosomes are isolated by gel extraction for example, from a 1% agarose gel.

Suitable gel extraction methods can be used including commercially available gel extraction kits such as the Zymo gel extraction kit. An exemplary protocol includes generally, follow the kit instructions, and preferably, wash twice with 400 μL of wash buffer. Perform a quick “dry spin” after the second wash to remove any residual wash buffer. Elute twice, each time with 9 μL of EB (18 μL total). To maximize yield, warm up elution buffer to 60-70° C. before adding it to the middle of the column filter.

In some forms, DNA is quantified, for example, with Qubit at this point. In preferred forms, the DNA amount should be at least 500 ng of gel-extracted and purified DNA per 5×10⁶ cells of input for a good RCMC result.

### Step VI. Library Preparation

In some forms, the stage of library preparation includes one or more steps of: (i) optional end polishing, (ii) streptavidin purification, (iii) end repair & A-tailing, (iv) adapter ligation, (vi) bead washing, (vii) test PCR run, (ix) pool PCR run, (x) sample purification, (xi) sample quantification, and (xii) pooling of barcoded samples.

### Optional End Polishing

In preferred forms, the end polishing step is skipped without detriment to the samples. In some forms, the end polishing step is assembled at room temperature, with one example of a reaction given below. For example, sample ends are polished and blunted again using the End-It enzyme reaction (Lucigen #ER81050). An exemplary end polishing reaction is shown in Table 9.

TABLE 9
End polishing reaction

| Stock | 25 μL |
|---|---|
| Input DNA | 17 μL |
| 10X End-it buffer | 2.5 μL |
| 10X ATP | 2.5 μL |
| 10X dNTP | 2.5 μL |
| End-it enzyme mix | 0.5 μL |

Incubate for 45 min at 25° C. (without mixing)
Inactivate the enzyme mix by incubating for 10 minutes at 65° C.

If an end polishing step is performed, in some forms, the reaction sample can be stored at 4° C. or −20° C. to proceed at a later time point, e.g., the following day.

### Ligated DNA Isolation (Streptavidin Purification)

Ligated DNA contact fragments are isolated by pulling down biotin-bound fragments. The end polished ligated DNA contact fragments are isolated by pulling down biotin-bound fragments using streptavidin, for example, using Dynabeads MyOne Streptavidin C1 or T1 beads. In some forms, streptavidin purification involves beads pre-wash and sample wash steps. In an exemplary form, the streptavidin purification involves the following steps:

### Beads Pre-Wash

An exemplary protocol bead pre-wash is as follows. First, prepare a fresh tube with 25-30 μL of C1 or T1 streptavidin beads per 5×10⁶ cells. Add 1 mL of 1× TBW to this tube to perform a couple wash steps prior to adding biotinylated sample to the beads. 250 mL of TBW includes: 2× BW (1×=125 mL) and 100% Tween-20 (R) (0.1%=250 μL); bring volume to 250 mL with ddH₂O and sterile filter. 2×BW for 250 mL includes NaCl (2 M=200 mL of 5 M NaCl stock); 1 M Tris-HCl pH 7.5 (10 mM=5 mL) and 0.5 M EDTA pH 8.0 (1 mM=1 mL

Using more beads for pulldown can improve yields, likely because smaller quantities of beads may get saturated (there are multiple biotin per Micro-C DNA fragment).

If carrying multiple identical 5×10⁶ cell input samples, combine them into a single tube and bring the tube volume to 300 μL with ddH₂O.

Nutate the beads and TBW mixture for 2 minutes, followed by the magnetic stand for 30-60 seconds. Carefully remove supernatant with a P200 tip attached to a P1000 tip without disturbing the beads. Resuspend the beads in 300 μL of 2× BW per sample tube.

Add the beads to the sample tubes in a 1:1 ratio. Mix the DNA and the beads for 20+ minutes at room temperature on a gentle nutator (overnight mixing also works well).

### Sample Wash Steps:

Spin briefly in a benchtop microcentrifuge and add 950 μL of 1× TBW. Invert the tube and shake at room temperature, 1200 RPM for 5 minutes on a mixer. Spin briefly in a bench microfuge and place on the magnet for 30-60 seconds. Carefully remove the supernatant and add 950 μL of 1× TBW to repeat the wash a second time. Spin briefly in a benchtop microcentrifuge, place on the magnet for 30-60 seconds and carefully remove the supernatant. With the tube on the magnetic stand, slowly rinse the beads with 10 mM Tris-HCl pH 7.5 without disturbing the beads. Remove 10 mM Tris once the End-Repair mix from the next step is ready. Preferably, never let beads dry out.

In preferred forms, Streptavidin T1 beads are used. In other forms, Streptavidin C1 or other beads are used. In further preferred forms, after the DNA sample is added washing steps do not involve any heating step or any washing solution of more than 30° C., more than 35° C., more than 40° C., or more than 45° C., or more than 50° C., or more than 55° C.

### End Repair, A-Tailing and Adaptor Ligation

Isolated ligated DNA samples are subjected to end repair and A-tailing using methods known in the art, for example, using commercially available kits (NEBNext Ultra II kit (New England BioLabs #E7645)). An exemplary end repair and A-tailing reaction is shown in

### Table 10.

TABLE 10
End repair and A-tailing reaction

| Stock | 60 μL |
|---|---|
| H₂O | 50 μL |
| End Prep Reaction Buffer | 7 μL |
| End Prep Enzyme Mix | 3 μL |

30 min @ 20° C. w/interval mixing
30 min @ 65° C.

In an exemplary form, the end repairing and A-tailing involves the following steps: Remove the 10 mM Tris from the beads above and resuspend them in the End Repair mix by either pipetting or vortexing. Make sure to avoid bubbles as this will reduce the efficiency of the end repair and A-tailing reaction. Incubate for 30 minutes at 20° C. with interval mixing at 1,000 RPM (1 minute on every 3 minutes off). Inactivate the enzymes by incubating for 30 minutes at 65° C. Transfer to ice.

An exemplary adapter ligation reaction is shown in Table 11.

TABLE 11
Adapter ligation reaction

| Stock | 96.5 μL | |
|---|---|---|
| Input DNA | 60 | μL |
| NEB Illumina adapter (first) | 2.5 | μL |
| Ligation Master Mix | 30 | μL |
| Ligation Enhancer (PEG 8000) | 1 | μL |

Vortex briefly and incubate for 30 minutes @ 20° C.
with interval mixing at 1,000 RPM (as before).

| | | |
|---|---|---|
| Add USER enzyme | 3 | μL |

Incubate for 15 minutes @ 37° C. with interval mixing at 1,000 RPM (as before).

In preferred forms, when the sample has 5 million cells or less per tube, half the volume of reaction mixtures in end repair, A-tailing, and adapter ligation is used (the only exception to halving is the amount of adapter, which is diluted based on the NEBNext kit's recommendations). The end repair, A-tailing, and adapter ligation steps preferably have the volumes specified in the NEBNext Kit protocol.

In the adapter ligation step, preferably, dilute the adapter in ddH2O or Tris pH 7.5-8.0 based on how much DNA is present. Use NEB's recommendations for the NEBNext prep. Also, the Ligation Master Mix and Enhancer can be mixed prior to addition to samples. The Ligation Master Mix is highly viscous, so take care to mix thoroughly with a pipette.

### Bead Wash

In some forms, bead washing includes one or more of the following steps:
i. Add 950 μL of 1× TBW, invert the tube, and mix at 1,200 RPM at RT for 3 minutes on a mixer.
ii. Spin the tube briefly, transfer to the magnetic stand, and carefully remove the supernatant.
iii. Repeat the 1× TBW wash (steps i and ii) a second time.
iv. Slowly rinse the beads with 10 mM Tris-HCl pH 7.5 without disturbing the beads (keep in magnetic stand).
v. Resuspend the beads in at least 20 μL of EB buffer (e.g., from the Zymo kit); you can do more, e.g., 50 μL or up to 100-120 μL depending on desired number of test PCRs and pool PCRs. Plan for 1 μL apiece per test PCR reaction and 15 μL apiece per pool PCR. In preferred forms, 2 test PCRs and 2-4 pool PCRs are run per sample depending on initial cell input, with higher cell inputs receiving more pool PCRs. Spreading bead-bound sample across multiple pool PCRs helps prevent streptavidin beads from saturating and inhibiting the amplification reaction.

Briefly vortex and spin to gather the sample-bound beads.

### Test PCRs

To determine the minimum number of PCR cycles to meet input material guidelines for capture or sequencing, a test library amplification is performed. In some forms, the reaction mixture is assembled in 200 μL PCR tubes on ice (e.g., 10 μL final reaction volume). In one form, the reaction mixture is as shown in Table 12.

TABLE 12
Test PCR run reaction mixture

| Stock | Total: 10 μL |
|---|---|
| Streptavidin beads (add last) | 1 μL |
| H₂O | 2 μL |
| 2X amplification mix (KAPA, Q5, etc.) | 5 μL |
| 10 μM universal primer | 1 μL |
| 10 μM index primer | 1 μL |

Mix the reaction by either pipetting up and down or vortexing.

In some forms when using low cell inputs (e.g., 1×10⁶ cells), 0.25×the primer volumes are used; if so, add 0.25 μL of each primer and 3.5 μL of water. In some forms, 2×KAPA HiFi Hot Start Mix or 2× Q5 NEB mix is used; if so, change the thermocycler program accordingly.

An exemplary Test PCR run is as follows: Prepare two test PCRs to test two different amplification amounts (e.g., 10 cycles and 13 cycles). Run the test PCR with the appropriate thermocycler conditions, as in Table 13. Add 3 μL of 10× Orange-G to each test PCR and load all of the sample on a regular 1-1.5% agarose gel side-by-side with 1 kb+ DNA ladder. Generally, there is no need to remove beads, they can stay in the well without disturbing DNA migration. In one form, the gel is run at 120 V for about 40 minutes.

Preferably, take a picture and calculate how much material has been obtained by amplification using the ladder for comparison. For example, a 13-cycle test PCR gel band is estimated at 250 ng total. 500 ng is needed for target enrichment (or 50 ng for regular Micro-C sequencing)—which is 2 times more the test PCR—but only 1/32^{nd} of the sample bead pool was used for this test run, meaning that the higher pool PCR input should necessitate fewer PCR cycles. So, 14 test PCR cycles would hit 500 ng and 14−log₂(32)=14−5=9 pool PCR cycles should hit 500 ng.

Also use this gel to check for the presence of adapter dimers (increase with more test PCR cycles) and primer dimers (decrease with more test PCR cycles). The presence of adapter dimers may necessitate an additional round of AmPure XP bead purification (below).

Exemplary PCR thermocycler conditions are as shown in Table 13.

TABLE 13
Test PCR thermocycler conditions

| | |
|---|---|
| Q5 NEB | KAPA HiFi |

| | | | | | |
|---|---|---|---|---|---|
| Start with | 98° C. | 30 | sec | 98° C. | 45 sec |
| 10-14 cycles | 98° C. | 10 | sec | 98° C. | 15 sec |
| | 65° C. | 1 | min | 60° C. | 30 sec |
| | | 15 | sec | | |
| | | | | 72° C. | 30 sec |
| Ending with | 65° C. | 5 | sec | 72° C. | 60 sec |

| | | | |
|---|---|---|---|
| 4° C. | On hold | 4° C. | On hold |

### Pool PCRs

In some forms, the reaction mixture is assembled in 200-μL PCR tubes in ice (50 μL total volume). An Exemplary pool PCR reaction is shown in Table 14.

TABLE 14
Exemplary pool PCR reaction

| Stock | Total: 50 μL | |
|---|---|---|
| Streptavidin beads (add last) | 15 | μL |
| H₂O | 0 | μL |
| 2X Q5 NEB mix | 25 | μL |
| 10 μM universal primer¹⁰ | 5 | μL |
| 10 μM index primer¹⁰ | 5 | μL |

An exemplary pool PCR protocol is as follows. Prepare 2-4 identical pool PCRs per sample, as previously determined. Mix the reaction by either pipetting up and down or vortexing. In preferred forms, no more than 10 pool PCR cycles are necessary to achieve 500 ng of sample library; in exemplary forms, 5-8 pool PCR cycles are sufficient to achieve 500 ng of sample library.

In some forms when using low cell inputs (e.g., 1×10⁶ cells), 0.25×the primer volumes are used; if so, add 1.25 μL of each primer and a total of 22.5 μL of streptavidin bead-bound sample and water for a total reaction volume of 50 μL.

In some forms, 2×KAPA HiFi Hot Start Mix or 2× Q5 NEB mix is used; if so, change the thermocycler program accordingly. Exemplary pool PCR thermocycler conditions are as shown in Table 15.

TABLE 15
Pool PCR thermocycler conditions

| | |
|---|---|
| Q5 NEB | KAPA HiFi |

| | | | | | |
|---|---|---|---|---|---|
| Start with | 98° C. | 30 | sec | 98° C. | 45 sec |
| 6-10 cycles | 98° C. | 10 | sec | 98° C. | 15 sec |
| | 65° C. | 1 | min | 60° C. | 30 sec |
| | | 15 | sec | | |
| | | | | 72° C. | 30 sec |
| Ending with | 65° C. | 5 | sec | 72° C. | 60 sec |

| | | | |
|---|---|---|---|
| 4° C. | On hold | 4° C. | On hold |

Although the C1 & T1 bead datasheets note that beads with similar chemistries (namely, M280 beads with the same core chemistry as T1 beads) can significantly inhibit PCRs when >75 μg of beads are present in a single reaction, 40 μL (300 μg) of both C1 and T1 beads have successfully tested in individual PCR reactions and found no noticeable inhibition. It should also be noted that T1 beads outperformed the C1 beads by 1.5 to 3-fold (about 1-2 cycles' worth of amplification). Thus, in preferred forms, T1 beads are used instead of C1 beads.

### Post-PCR Processing

In some forms, one or more steps are carried out post-PCR:
1. Transfer all of the PCR reaction to a low-retention 1.5 mL tube. Combine any identical pool PCRs into the same sample tube.
2. Place the tube on a magnetic rack and allow the streptavidin beads to move to the side of the tube until the liquid is clear.
3. Move the liquid to a new low-retention 1.5 mL tube and proceed.
   In some forms, the AmPure and streptavidin beads are mixed together. In other forms, for better bead separation, removal of streptavidin beads is done before adding the cleanup beads.

### 0.9×AmPure XP Beads Purification

In some forms, AmPure XP beads purification includes one or more of the following steps:
(i) Allow the AmPure XP beads to equilibrate to RT and mix them well;
(ii) For each 50 μL of post-PCR sample, add 45 μL of beads and pipet up and down until thoroughly resuspended;
(iii) Incubate at RT for 15 min to allow the DNA to bind to beads;
(iv) Place the tube onto the magnetic rack and incubate until solution is clear;
(v) Remove and discard the supernatant without disturbing the beads;
(vi) Add 200 μL of freshly prepared 80% EtOH without disturbing the beads;
(vii) Incubate the solution for 30 seconds while still on the magnetic rack;
(viii) Remove and discard the EtOH without disturbing the beads;
(ix) Repeat the EtOH wash steps (vi-viii);
(x) Remove residual ETOH using a P20 and/or vacuum without disturbing the beads;
(xi) Air-dry the beads for 5 minutes (do not overdry the beads!);
(xii) Remove the tube from the magnetic rack & add at least 25 μL of elution buffer or water to the sample. The aim is to have a final concentration of DNA between 4 and 20 nM (in theory, you should use 50 μL for 1000 fmol to get 20 nM, but one should account for 50% of material loss);
(xiii) Thoroughly re-suspend the beads by pipetting up and down 10-20×;
(xiv) Incubate at RT for 2 minutes to allow DNA to elute off of the beads;
(xv) Place the tube back on the magnetic rack and incubate until the solution is clear and;
(xvi) Transfer the supernatant to new low-bind tube.

In some forms, two or more consecutive rounds of AmPure cleanup to ensure contaminating adapter dimers are eliminated.

Qubit and qPCR Quantify Samples.

In some forms, sample is quantified. Preferably, the library fragment distribution is determined using a Fragment Analyzer.

### Pool Barcoded Samples

The sample can then be submitted for sequencing if genome-wide Micro-C is desired. For the RCMC disclosed herein, the sample is subject to target enrichment for Region Capture Micro-C.

In some forms, barcoded samples can be pooled in a desired ratio. In some forms, the barcoded samples can be pooled using standard pooling, which is done at a 1:1 ratio so that each library is sequenced equally. In some forms, if libraries are desired to be sequenced in different amounts, they may be pooled according to the desired proportions. For example, if four samples are being pooled and higher depth is preferred for one of them, they may be pooled in a 2:1:1:1 or another ratio. In preferred forms, barcoded samples are pooled at a 1:1 molar ratio.

If doing regular Micro-C:
Consider using ˜1 pmol total DNA, splitting this amount between barcoded samples (e.g., 5 samples, 200 fmol each; 20 samples, 50 fmol each).
Determine the sequencing technology you would like to use and its associated requirements (sample volume & concentration). Paired-end sequencing with 35-150 bp read lengths is recommended.
   If doing Region Capture Micro-C:
In general, any of several commercially available target enrichment strategies can be used to pull down regions of interest.
The Twist Biosciences's Target Enrichment Protocol is merely provided herein as an example. The protocols provided by Roche, Agilent, or other target enrichment protocols can be used here.

### B. MNase Titration Protocol

As discussed above, the RCMC workflow relies on digesting chromatin to primarily mononucleosomally-sized (e.g., 150-200 bp) fragments. Over-digestion of chromatin leads to short fragments and poor proximity ligation, whereas under-digestion risks mapping fewer contacts than possible due to the lower number of mono-nucleosomes available for ligation.

Digestion time and conditions may vary from cell-type to cell-type and can vary by fixation. In preferred forms, an MNase titration is carried out to determine the ideal MNase digestion amounts and conditions before proceeding with the rest of the protocol.

Conduct an MNase titration for each new batch of crosslinked cells.

In some forms, the MNase Titration Protocol includes one or more stages of (I)-(III). In preferred forms, the MNase Titration Protocol includes stages of (I)-(III).
(I) Prepare crosslinked chromatin from cells,
(II) Digest crosslinked chromatin with Micrococcal Nuclease (MNase), and
(III) Isolate DNA and visualize the titration.
   (I) Prepare Crosslinked Chromatin from Cells

In preferred embodiments, the stage of preparing crosslinked chromatin from cell culture involves the steps described under RCMC Protocol, Pre-Capture.

(II) Digest Crosslinked Chromatin with Micrococcal Nuclease (MNase)

In some forms, one of more of the following steps are performed to digest crosslinked chromatin with MNase.
1. Prepare fresh, “complete” MB #1 solution. An exemplary formulation for “complete” MB #1 solution is provided in Table 16/

TABLE 16
“complete” MB#1 solution

| Stock | 5 mL | 10 mL | Final |
|---|---|---|---|
| MB#1 | 4850 μL | 9700 μL | 50 mM NaCl, 10 mM Tris, 5 |
| 10% NP-40 | 100 μL | 200 μL | mM MgCl₂, 1 mM CaCl₂, 0.2% |
| 100x PIC | 50 μL | 100 μL | NP-40, 1X PIC. |
| | | | Use protein-grade NP-40 \| make |
| | | | sure to use NP-40 Alternative, not |
| | | | NP-40. |
| | | | Protease inhibitor cocktail |
| | | | dissolved in MB#1, aliquoted, and |
| | | | stored in −20° C. |

2. Thaw a cell pellet of the desired size. In some forms, a cell pellet containing about 1 or 5×10⁶ cells is placed on ice and resuspended in complete MB #1 to a concentration of about 1×10⁶ cells/100 μL. Incubate for 20 min on ice. Centrifuge at ˜1,750×g for 5 min at 4° C. Discard supernatant, leaving ˜10-20 μL behind. In preferred embodiments, supernatant removal should be done with a P200 pipet tip; for larger volumes, append the tip to a P1000 tip.
3. Wash nuclei pellet in complete MB #1. In preferred forms, the nuclei pellet is washed in complete MB #1 at a concentration of 1×10⁶ cells/100 μL, resuspending the pellet up & down with a pipette. If performing the titration on 1×10⁶ cells with a 5×10⁶ cell input, aliquot 100 μL of cells to 5×low-retention 1.5 mL Eppendorf tubes. Centrifuge at ˜1,750×g for 5 min at 4° C. Discard supernatant with a P200 pipette tip, removing as much liquid as possible without disturbing the pellet. Thaw MNase; partially used aliquots are stored at −20° C. (use no more than 3 times), fresh aliquots are stored at −80° C. If using 1×10⁶ cell aliquots or if pipetting larger volumes is preferred, prepare a 1:20 dilution in 10 mM Tris-HCl, pH 7.5 (2 μL MNase+38 μL buffer).
4. Resuspend nuclei pellet in complete MB #1, preferably at a concentration of 1×10⁶ cells/100 μL, pipetting up & down.
5. Add increasing amounts of MNase. An exemplary MNase titration is given below:

If digesting 1×10⁶ cell aliquots:
2 U: 2 μL of a 1 U/μL MNase dilution
4 U: 4 μL of a 1 U/μL MNase dilution
7 U: 7 μL of a 1 U/μL MNase dilution
12 U: 12 μL of a 1 U/μL MNase dilution
21 U: 21 μL of a 1 U/μL MNase dilution

If digesting 5×10⁶ cell aliquots:
10 U: 0.5 μL of the 20 U/μL MNase stock
20 U: 1 μL of the 20 U/μL MNase stock
35 U: 1.75 μL of the 20 U/μL MNase stock
60 U: 3 μL of the 20 U/μL MNase stock
105 U: 5.25 μL of the 20 U/μL MNase stock

The target is to digest chromatin to an 80-90% monomer/20-10% dimer ratio, with a faint yet detectable (<5%) trimer percentage. Briefly vortex the tubes and incubate for 20 min at 37° C. with shaking at 1000 RPM. Prepare a 1.5% agarose gel.
6. Transfer the nuclei back onto ice and promptly stop the reaction by adding 500 mM EGTA to a final concentration of 4 mM (0.8 μL for 100 μL of sample). Vortex briefly to mix. Incubate for 10 min at 65° C. to ensure complete inactivation of the MNase. Centrifuge at 1,750-2,000×g for 5 min at 4° C. Discard supernatant with a P200 tip.

### (III) Isolate DNA and Visualize the Titration

7. Resuspend each pellet in Reverse Crosslinking solution and resuspend up and down. Reverse crosslinks for a minimum of 2 hours to overnight at 65° C. The components of an exemplary reverse crosslinking reaction is provided in Table 17. In some forms, a pre-mix of these reagents can be made just before the addition.

TABLE 17
Reverse crosslinking reaction

| | 5 × 10⁶ | | 1 × 10⁶ | | | |
|---|---|---|---|---|---|---|
| | cells | | cells | | | |
| Stock | 265 μL | | 133 μL | | Final | |
| Tris-EDTA (TE) buffer | 200 | μL | 100 | μL | | |
| (first) | | | | | | |
| 20 mg/mL | 26 | μL | 13 | μL | 2 mg/mL | |
| Proteinase K | | | | | Proteinase K | |
| 10% SDS solution | 26 | μL | 13 | μL | 1X SDS | |
| 5M NaCl | 10.4 | μL | 5.2 | μL | 250 | mM |
| 10 mg/mL RNaseA | 2.6 | μL | 1.3 | μL | 0.1 | mg/mL |

Incubate at 65° C. overnight while shaking at 1,000 RPM.

8. Optional Phenol-Chloroform: Isoamylic alcohol (PCI) extraction. Preferably, all steps are performed in the fume hood.
   i. Bring sample volumes to 200 μL (add 50 μL) with 1× TE, and transfer sample to labelled phase-lock tubes. A quick spin may be necessary pre-transfer to remove gel from the tube lid.
   ii. Add 200 μL of PCI to the sample volume. PCI often comes with a top “containment” layer of liquid-avoid pipetting from this top layer and instead pipette from the PCI solution beneath it.
   iii. Vortex for 20 seconds and then spin for 15 minutes at 19,800×g at room temperature.
   iv. Transfer the upper layer (˜150 μL) to a new low-retention tube.
9. ZymoClean: DNA purification with the Zymo DNA Clean & Concentrator (e.g., DCC-25) kits. DNA purification kits are commercially available and any other DNA purification kits can be used in the disclosed methods.
   i. Add 5 volumes (1 mL) of DNA binding buffer and load to the column (you may have to load the column multiple times to load all of the sample).
   ii. Wash twice with 400 μL of wash buffer. Perform a quick “dry spin” after the second wash to remove any residual wash buffer.
   iii. Elute with 25 μL of elution buffer (EB).
10. Quantify the DNA by Nanodrop; this should help avoid “overloading” a gel well. For example, loading 1-5 μg of DNA per well of a standard 10-well gel should be good, but >7 μg may lead to gel running issues.

Add 10× loading dye to the samples. Using Orange G as a dye will minimally interfere with the size of the nucleosomal fragments.

Load the samples onto the 1.5% agarose gel; also load 1 kb+ DNA ladder.

Run the gel (e.g., 120V for 60 min) and visualize it using a gel imager. Pick the sample with the ideally digested ratio of monomers to dimers (a faint trimer band should be visible, while a tetramer band should not be visible).

### C. Capture Protocol

The RCMC method applies in general to any combination of probe panels and regions, regardless of the target enrichment strategy being used. In some forms, target enrichment is performed following the hybridization protocol of any major DNA synthesis company. In preferred forms, Capture is performed following the Standard Hybridization Target Enrichment Protocol provided by Twist Bioscience.

In some forms, one or more of the following exceptional steps are performed:
Exceptional Step 1: Conducting a test PCR to confirm how many amplification cycles are necessary. In some forms, fewer cycles than recommended are necessary e.g., capturing 3 Mb total from a 2-4 μg capture input should meet sequencing submission requirements in 6-7 cycles for most sequencing platforms.
Exceptional Step 2: utilizing the full bead-bound sample for the pool PCR instead of half, and split it across two identical pool PCRs.
Exceptional Step 3: when performing Capture on non-human samples, Twist's Blocker Solution (which is human-specific) is replaced with Cot-1 DNA specific to the species of interest to similarly prevent nonspecific binding to probes.
Exceptional Step 4: In some forms, multiple Capture panels are combined (e.g., three 1-Mb panels) e.g., the multiple Capture panels are combined, vacuum dried, and resuspended in the protocol-specified volume before proceeding.

### Probe Design

Probe design is an important determinant of the success for an RCMC experiment, as poorly designed probe panels may suffer from significant off-target pulldown, poor Capture efficiency, and/or be wasteful of sequencing costs. An exemplary protocol for designing probes includes the following steps:
Step 1: the coordinates of the region(s) of interest are determined. This includes utilizing architectural and regulatory relationships of interest (e.g., known enhancer-promoter pairs, key genes with incompletely characterized regulation, genomic structures of interest) and extant datasets (if available, e.g., ChIP-seq for epigenetic markers of regulatory features, ATAC-seq, RNA-seq) to define the coordinates. In some forms, regions a few hundred kb in length are chosen. In preferred forms, regions span 1-5 Mb in size, thus allowing most contact fragments associated with any given point within the region of interest to have their contact pair mate fragment also lie within the region.
Step 2: Contact Twist Biosciences with an interest in designing probes for a region. Ask for a probe tiling for the region. In some forms, single-stranded probes and shorter (e.g., 70 bp) or longer (e.g., 120 bp) probes are used and tiled with some degree of overlap. In preferred forms, double-stranded DNA 80-mer probes are tiled end-to-end with no overlap or gaps across the entire locus. Utilize repeat masking to remove probes likely to capture repetitive regions.
Step 3: Double-check the tiling to confirm that no important sites within the region (e.g., active genes, epigenetic marker peaks, CTCF binding sites) have been excluded. Add specific sets of probes back into the design to recover any missing coverage that is desirable. A final probe coverage of ˜70-85% of the locus is an expected outcome following repeat masking, depending upon the number of repeat elements in the region.
Step 4: Finalize the design and order the probes and associated Capture reagents.

In preferred form, Twist Biosciences is used to order capture probes. In other forms, other DNA synthesis and hybridization companies are used.

The invention will be further understood in view of the following non-limiting examples.

### EXAMPLES

### Materials and Methods

### Experimental Procedure

### Overview of the RCMC Experiment

RCMC was developed by merging Micro-C¹² with tiling region capture of a locus^{28,29} including innovative additional steps that significantly improved the results obtained with RCMC when compared to Micro-C¹² alone or in combination with tiling region capture of a locus^{28,29}. An overview of the RCMC protocol is provided herein, and a detailed protocol is provided as above in the “Detailed Description of the Invention”. The data generated herein ome from merging of two RCMC biological replicates for each of the five tested conditions (WT, transcriptional inhibition for 45 min or for 4 h, cohesin depletion and a cohesin depletion control). For four of the tested conditions (all except transcriptional inhibition for 4 h), the first biological replicate is a compilation of three technical replicates generated from the same batch of harvested cells. Biological replicates were generated by harvesting (culturing, crosslinking, aliquoting, and snap-freezing) 125-200 M cells for each tested condition, after which downstream RCMC steps (Micro-C and Capture) were applied to five snap-frozen 5 M cell aliquots to generate one to three technical replicates for each biological replicate. Reaction volumes for performing RCMC on both 1 M and 5 M cell samples are provided above in the “Detailed Description of the Invention”.

### Cell Culture

mESCs (JM8.N4 mESCs52; Research Resource Identifier: RRID: CVCL_J962; obtained from the KOMP Repository at UC Davis) were cultured at 37° C. with 5% CO₂ on plates coated with 0.1% gelatin solution (Sigma-Aldrich #G1890) under feeder-free conditions in medium consisting of KnockOut DMEM (ThermoFisher #10829-018) with 15% FBS (HyClone, SH30396.03, lot no. AE28209315) and 1,000 U/mL LIF (homemade⁵³), 1 mM MEM Non-Essential Amino Acid Solution (ThermoFisher #11140-050), 2 mM GlutaMAX (ThermoFisher #35050061), 100 μg/mL penicillin-streptomycin (ThermoFisher #15140-122) and 0.1 mM 2-mercapocthanol (ThermoFisher #31350010) supplemented with 2i: 10 μM MEK inhibitor (Tocris #PD0325901) and 3 μM GSK inhibitor (Sigma-Aldrich #SML1046). FIM RAD21-mAID-BFP-V5 JM8.N4 mESCs were previously generated and validated in the laboratory³⁸. mESCs were fed daily by replacing half of the medium and passaged every 2 days with TrypLE Express Enzyme (ThermoFisher #12605036). One day before treatment and harvesting, cells were swapped to medium as described above without 2i.

HEK293T cells were obtained from ATCC (CRL-3216) and were cultured at 37° C. with 5% CO₂ in DMEM supplemented with 10% FBS, 2 mM L-glutamine, 1×penicillin-streptomycin and 0.5 mM β-mercaptocthanol.

### Depletion of Cohesin

Depletion of cohesin was achieved using indole-3-acetic acid (IAA) treatment of the cell line clone FIM RAD21-mAID-BFP-V5 as previously described^{13,38}. A 250 mM IAA (BioAcademia #30-003-10) stock was prepared by dissolving the drug in DMSO. FIM RAD21-mAID-BFP-V5 JM8.N4 mESCs³⁸ were grown to ˜80% confluency in medium as described above, with a swap to 2i-free medium 24 h before treatment. Cells were washed once with PBS and fed fresh 2i-free medium containing either only DMSO (untreated control) or 500 μM IAA (cohesin depleted), incubated for 3 h and then harvested.

### Inhibition of Transcription

Inhibition of RNA Pol II activity was achieved using triptolide treatment as previously described¹². A 1 mM triptolide (Sigma-Aldrich #T3652) stock was prepared by dissolving the drug in DMSO. WT JM8.N4 mESCs52 were grown to ˜80% confluency in medium as described above, with a swap to 2i-free medium 24 h before treatment. Cells were washed once with PBS and fed fresh 2i-free medium containing 1 μM triptolide, incubated for 45 min or for 4 h and then harvested.

### Crosslinking

Cells were doubly crosslinked to fix protein-protein and protein-DNA interactions using DSG (disuccinimidyl glutarate, 7.7 Å) (ThermoFisher #20593) and formaldehyde (ThermoFisher #28906), respectively. Crosslinking medium was prepared by diluting freshly made DSG stock solution (300 mM DSG in DMSO) to 3 mM in 1× PBS (ThermoFisher #10010031). Trypsinized cells were resuspended to single cells, counted, washed in PBS, and then resuspended in crosslinking medium at a concentration of 1 M cells/mL. The crosslinking reaction was gently mixed at room temperature for 35 min, after which formadehyde was added to a final concentration of 1%. The double crosslinking reaction was mixed at room temperature for an additional 10 min before quenching with Tris buffer pH 7.5 (K-D Medical #RGE-3370) at a final concentration of 0.375 M. Treatments for non-WT samples (1 μM triptolide, 500 μM IAA or DMSO) were added to all harvesting reagents used before Tris quenching (PBS, trypsin, trypsin-quenching media, and crosslinking medium) to avoid post-treatment rescue during the crosslinking process. Crosslinked cells were washed twice with 1× PBS, recounted to quantify any sample loss during fixation and then partitioned into 5 M cell aliquots that were pelleted and snap-frozen in liquid nitrogen for storage at −80° C.

### MNase Titration

Digesting the crosslinked genome to the nucleosome-sized fragments (150-200 bp) necessary to capture nucleosome-resolution DNA contacts requires a titration to identify the ideal MNase digestion concentration and reaction conditions. Accordingly, MNase titrations were performed for each batch of crosslinked cells before performing the RCMC protocol. The titration involved MNase digestion of 1 M or 5 M cell samples varying MNase concentrations, reversal of crosslinks, DNA purification, and gel-based separation to visualize the distribution of fragment sizes (see corresponding sections below). Ideal digestion concentrations were identified by samples digested to primarily (˜80%) mononucleosomal fragments (150-200 bp), few (˜15-20%) dinucleosomal fragments (200-350 bp) and a faint but visible band (<5%) of trinucleosomal fragments (400-500 bp) (FIG. 6A).

### MNase Digestion

Cell membranes were solubilized to extract intact nuclei by resuspending crosslinked 5 M cell pellets in Micro-C Buffer #1 (MB #1; 50 mM NaCl, 10 mM Tris-HCl pH 7.5, 5 mM MgCl₂, 1 M CaCl₂), 0.2% NP-40 Alternative (Millipore Sigma-Aldrich #492018), 1× Protease Inhibitor Cocktail (Sigma-Aldrich #5056489001)) at 1 M cells per 100 μl for 20 min on ice. Following an MB #1 wash, samples were resuspended in 100 μl MB #1 and the ideal amount of 20 U/μL MNase (Worthington Biochem #LS004798) determined by the MNase titration was added. This digestion reaction was mixed at 37° C. for 20 min on a thermomixer before being quenched with 4 mM EGTA (bioWORLD #40520008) and heat inactivated at 65° C. for 10 min. Digested nuclei were washed twice with ice-cold Micro-C Buffer #2 (50 mM NaCl, 10 mM Tris-HCl pH 7.5, 10 mM MgCl₂, and 100 μg/mL BSA (Sigma-Aldrich #B8667)).

### End Repair and Labelling

To generate blunt ends on digested DNA fragments before proximity ligation and add biotinylated nucleotides, a series of enzymatic processing steps were performed. First, to catalyze the addition of 5′-phosphate groups and the removal of 3′-phosphate groups, digested samples generated from 5 M cell inputs were incubated in end-repair reactions (50 U T4 Polynucleotide Kinase (New England BioLabs #M0201), 50 mM NaCl, 10 mM Tris-HCl pH 7.5, 10 mM MgCl₂, 100 μg/mL BSA, 2 mM ATP (ThermoFisher #R1441), and 5 mM DTT (Sigma-Aldrich #10197777001), in water) at 37° C. for 15 min while mixing. To create 5′ fragment overhangs for end-blunting and labelling, 50 U DNA Polymerase I Klenow Fragment (New England BioLabs #M0210) was added to the reaction and incubated at 37° C. for 15 min while mixing. Next, a mixture of dNTPs in end-labelling buffer (66 μM each of dTTP (Jena Bioscience #NU-1004), dGTP (Jena Bioscience #NU-1003), biotin-dATP (Jena Bioscience #NU-835-BIO14), and biotin-dCTP (Jena Bioscience #NU-809-BIOX), 1× T4 DNA Ligase Buffer, 100 μg/mL BSA, in water) was added to the reaction. This reaction was incubated at room temperature for 45 min with interval mixing before being quenched by 30 mM EDTA (Invitrogen #15575020) and heat inactivated at 65° C. for 20 min. Finally, end-blunted and biotin-labelled nuclei were washed once with Micro-C Buffer #3 (50 mM Tris-HCl pH 7.5, 10 mM MgCl₂, and 100 μg/mL BSA).

### Proximity Ligation and Removal of Unligated Biotin

Proximity ligation was performed by incubating labelled chromatin in a ligation reaction (10,000 U T4 DNA Ligase (New England BioLabs #M0202), 1× T4 DNA Ligase Buffer, 100 μg/mL BSA, in 500 μL water) at room temperature for at least 2.5 h with gentle mixing. To remove biotinylated dNTPs from all unligated fragment ends, samples were digested by 1,000 U Exonuclease III (New England BioLabs #M0206) in reaction buffer (1× NEBuffer #1 in water) at 37° C. for 15 min with interval mixing.

### DNA Purification and Size-Selection

Reverse crosslinking: To prepare ligated DNA for library generation, DNA was reverse crosslinked, and proteins and RNA were digested by adding 1% SDS (Sigma-Aldrich #L3771), 2 mg/mL Proteinase K (Viagen Biotech #501-PK), 250 mM NaCl and 100 μg/mL RNaseA (ThermoFisher #EN0531) to the samples and incubating at 65° C. overnight. DNA was extracted using phenol: chloroform: isoamylic alcohol (Sigma-Aldrich #P2069) in a 1:1 volumetric ratio using 5PRIME Phase Lock Gel Light tubes (Quantabio #2302820). The aqueous phase was further purified using the Zymo DNA Clean & Concentrator kit (Zymo Rescarch #D4034) according to the kit manual.

Gel extraction: Dinucleosome-sized DNA fragments (200-350 bp) were isolated by extraction from a 1% agarose gel (VWR #97062) (FIG. 6B). Gel extracts were purified using the Zymo Gel Purification kit (Zymo Research #D4008), and samples were quantified by Qubit 1× dsDNA High Sensitivity Assay (Invitrogen #Q33231). Sample ends were polished and blunted again using the End-It enzyme reaction (Lucigen #ER81050) at 25° C. for 45 min, followed by reaction inactivation at 65° C. for 10 min.

Ligated fragment pulldown: Ligated DNA contact fragments were isolated by pulling down biotin-bound fragments using Dynabeads MyOne Streptavidin T1 beads (Invitrogen #65601). DNA samples were bound to beads in a Binding and Wash Buffer (1 M NaCl, 5 mM Tris-HCl pH 7.5, 500 μM EDTA) at room temperature for at least 30 min with mixing. After two washes with Binding and Wash Buffer containing 0.1% Tween-20 (Sigma-Aldrich #P8074), the bead-bound samples were washed once with 10 mM Tris-HCl pH 7.5 before library preparation.

### Library Preparation

Illumina library preparation was performed using the NEBNext Ultra II kit (New England BioLabs #E7645) to end-repair, A-tail, and adapter ligate the bead-bound samples. All steps were performed as directed by the manual, except those incubations included interval shaking (1 min on, 3 min off) at 1,000 RPM. Sample washes were performed using Binding and Wash Buffer with 0.1% Tween-20 (Sigma-Aldrich #P8074) and 10 mM Tris-HCl pH 7.5 washes. To determine the minimum number of PCR cycles to meet input material guidelines for capture or sequencing, a test library amplification was performed with 5% or less of the prepped library to quantify the yield. The test PCR reaction mixture was run on an agarose gel and yield was quantified using image quantification software Image Studio Lite (LI-COR Biosciences). Ten or fewer PCR cycles to meet capture input requirements is should be used, to reduce PCR duplicates, and the RCMC replicates herein used seven to eight PCR cycles for final library amplification. All library amplifications were done using sequencing indices from the NEB Multiplex Oligos for Illumina Primer Set 1 (New England BioLabs #E7335) and the KAPA HiFi HotStart ReadyMix enzyme (Roche #07958927001). Following library amplification, the T1 Dynabeads containing the original bead-bound samples were removed and the amplified libraries were purified to remove adapter dimers, primers, and contaminants using AmPure XP beads (Beckman Coulter #A63880). Purified libraries were quantified via Fragment Analyzer and qPCR at the MIT BioMicro Center to determine library concentrations for pooling before capture.

### Capture Probe Design

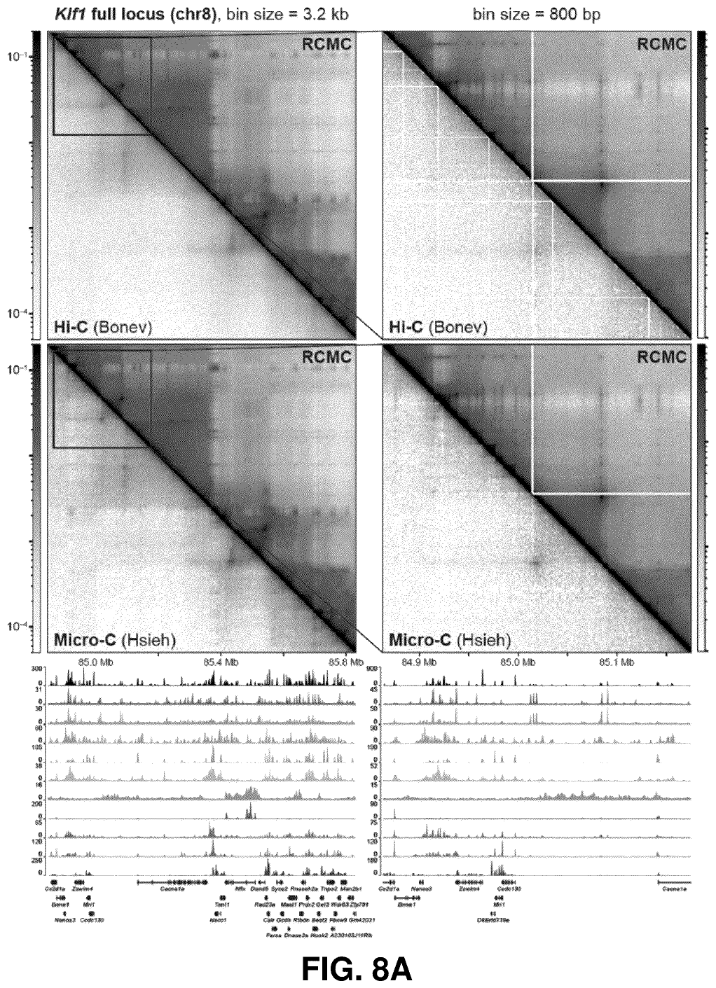

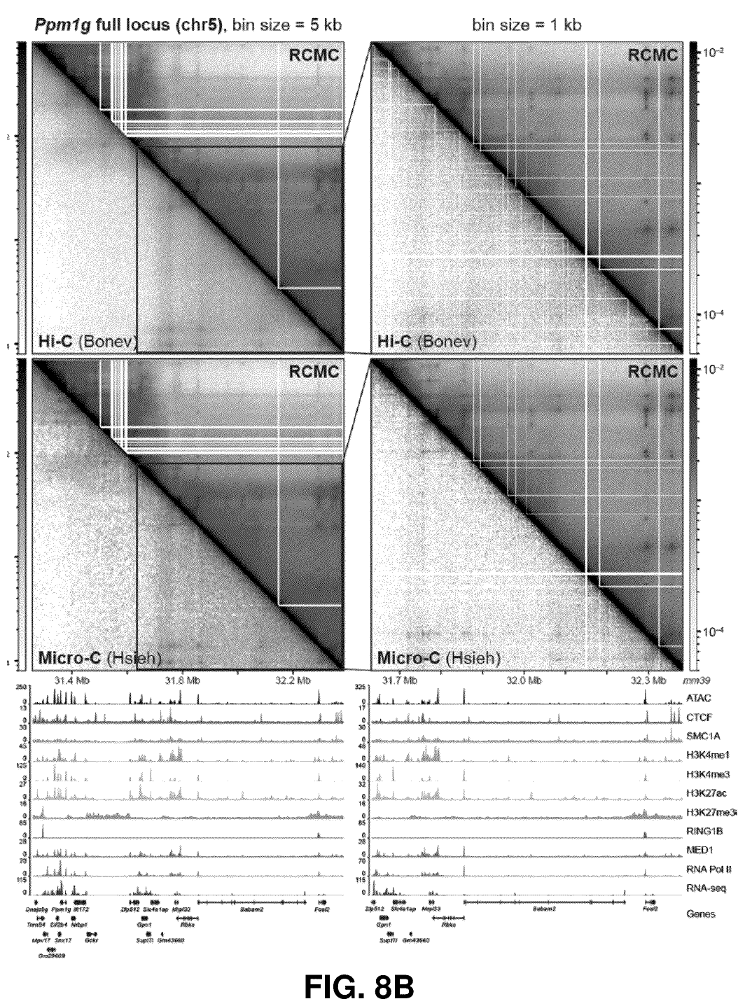

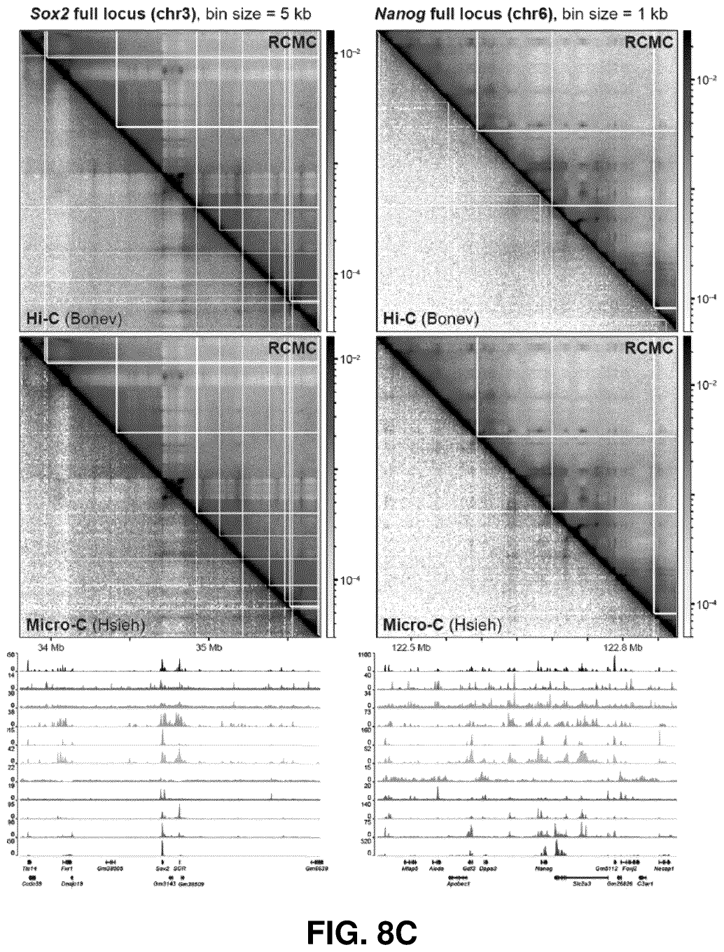

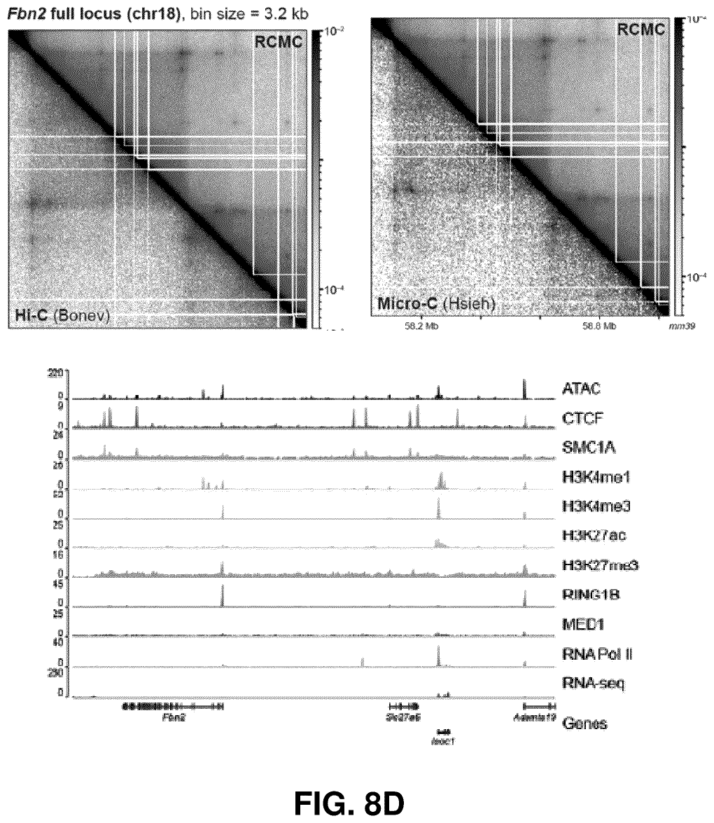

Target loci of interest were identified based on genomic features or E-P relationships of interest. Klf1 and Ppm1g were selected as gene-rich loci, Fbn2 was selected as a gene-poor control with a well-established CTCF- and cohesin-mediated loop³⁸, and Sox2 and Nanog were later selected as loci for comparing RCMC against TMCC (FIG. 8). Using the UCSC Genome Browser and HiGlass visualization of existing mESC 3C datasets, locus bounds were selected to include visible local structures and genomic features in roughly 1-Mb-sized regions. Once loci had been selected, 80-mer probes were designed to tile end-to-end without overlap across the capture loci through Twist Bioscience (FIG. 6C). Probes with high predicted likelihoods of off-target pull-down (for example, such as those in high-repeat regions) were masked and removed from the probe tiling, and probe coverage was double-checked to ensure the inclusion of key genomic features (for example, all promoters and CTCF sites in the locus) before finalization. Probe panels were synthesized and purchased as Custom Target Enrichment Panels from Twist Bioscience.

### Capture of Target Loci

Capture was performed in accordance with Twist Bioscience's Standard Hybridization Target Enrichment Protocol. Briefly, pooled sample libraries were dried and mixed with Hybridization Mix (Twist Bioscience #104178), Custom Panels (Twist Bioscience #101001) and Universal Blockers (Twist Bioscience #100578), as well as Mouse Cot-1 DNA (Invitrogen #18440016). The library pool was hybridized to the biotinylated probe panel overnight, after which streptavidin beads (Twist Bioscience #100983) were used to pull down probes with hybridized ligated fragments and then washed (Twist Bioscience #104178) to remove unbound fragments. Another round of PCR amplified the target-enriched library using the Equinox Library Amplification Mix (Twist Bioscience #104178), including a test PCR (as described above) to identify the number of amplification cycles necessary to meet sequencing requirements. With 2-4 μg input library for capture, the RCMC samples generated herein needed five to six cycles of post-capture PCR amplification. Following PCR amplification, the captured library was purified (Twist Bioscience #100983) and then quantified via both Fragment Analyzer and qPCR at the MIT BioMicro Center in preparation for sequencing submission.

Three technical replicates of the pre-capture Micro-C library were generated for each of the four initially tested conditions (WT, 45 min transcriptional inhibition with triptolide, RAD21 depletion and a RAD21 depletion control), after which each replicate was simultaneously captured for the Klf1, Ppm1g and Fbn2 loci. After the publication of TMCC¹⁷, additional probes for the Sox2 and Nanog loci were designed and a single additional capture experiment was conducted pooling all three pre-capture Micro-C libraries for simultaneous Sox2 and Nanog capture. Subsequently, a biological replicate was generated for each of the initially tested conditions, with the inclusion of the additional perturbation of 4 h transcriptional inhibition with triptolide. Pre-capture Micro-C libraries were constructed for each of the five conditions, after which each library was simultaneously captured for all five target loci. Finally, a biological replicate of the 4 h transcriptional inhibition perturbation was generated; once a pre-capture Micro-C library for it was generated, it was pooled with the WT library from the first technical replicate of the first biological replicate, and the pooled libraries were simultaneously captured for all five target loci.

### Sequencing

Following qPCR quantification, post-capture libraries across samples (WT, transcriptionally inhibited, cohesin depleted, and DMSO-treated control) were pooled in a 1:1 molar ratio. Pooled libraries were sequenced by paired-end 2× 50 cycle sequencing kits with Illumina NovaSeq SP or SI flow cells on a NovaSeq 6000 system by the Broad Institute of MIT and Harvard's Walk-Up Sequencing services. Basecalls for NovaSeq output were performed using bcl2fastq v2.20.0.422.

### Data Analysis

### Mapping and Normalizing RCMC

RCMC paired-end reads generated by the Illumina NovaSeq sequencers were downloaded as .fastq files for each sample, pair mate, and flow cell lane. Read quality was verified using FastQC (v0.11.9). Paired end reads were aligned to the UCSC mm39 genome using bowtie2 (v2.3.5.1) with-local-reorder-very-sensitive-local. Aligned paired-end reads were then parsed with pairtools (v0.3.0) parse with-add-columns mapq-walks-policy mask-min-mapq 2. Parsed reads were filtered for PCR duplicates and unmapped/multiple mapping reads with pairtools dedup with --max-mismatch 1. Remaining reads were indexed (pairix v0.3.7) and filtered (pairtools select) to retain only those reads where both read mates lay in a locus of interest. These filtered reads were subsequently converted to .cool format using cooler (v0.8.11) cload pairs, creating binned read counts across the genome for 50-bp bins. Finally, .cool files were converted to the .mcool format with cooler zoomify including the—balance option, compiling read counts for bins from 50 bp up to 10 Mb in size.

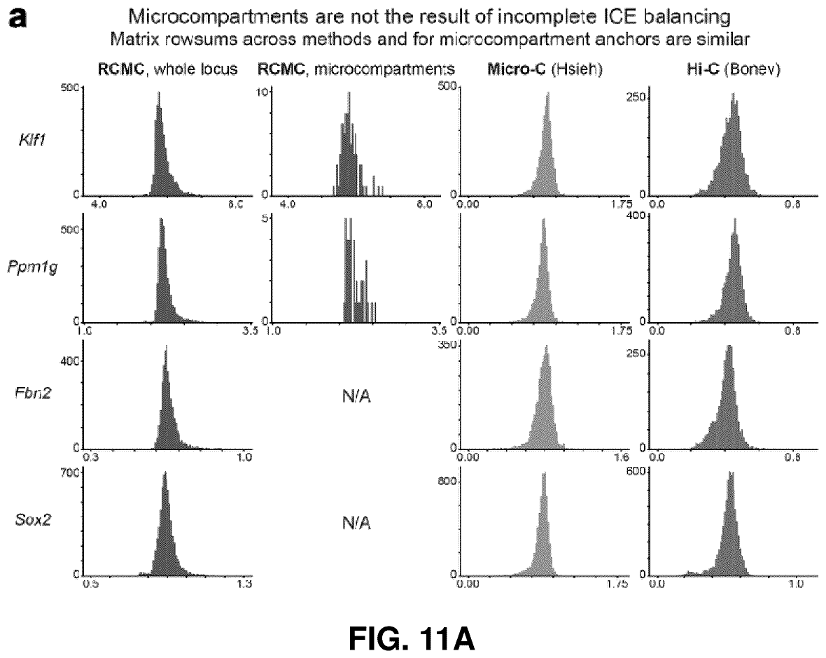

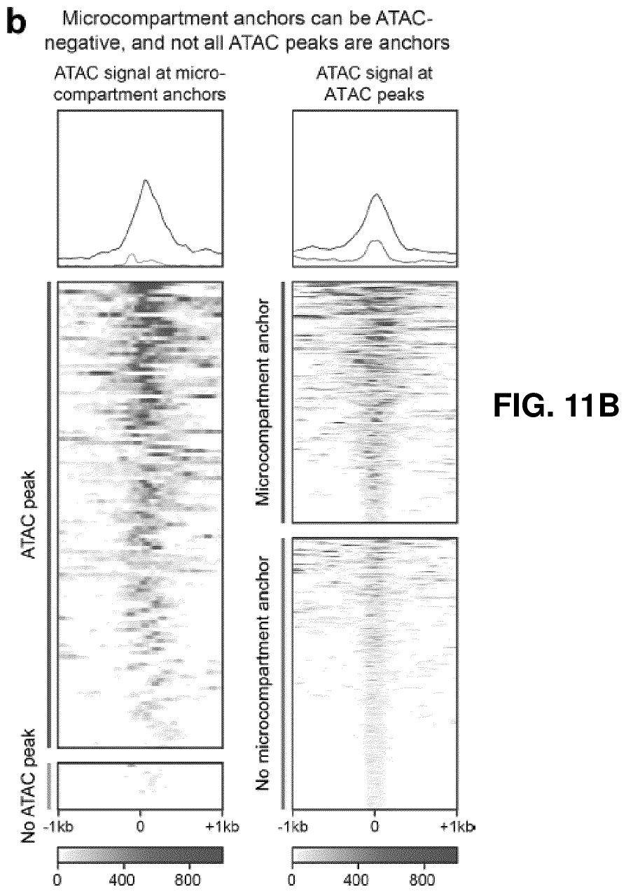

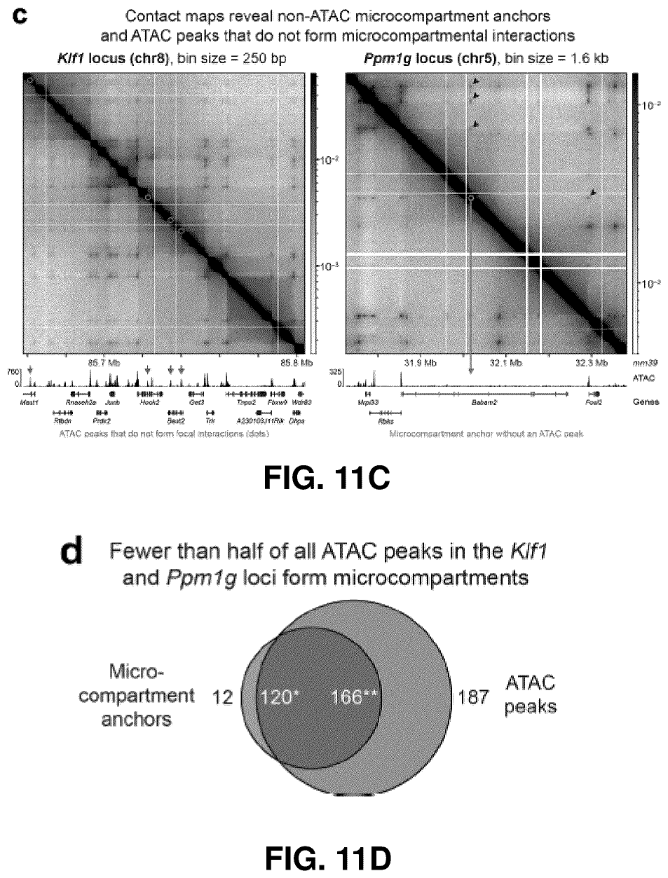

Contact matrices were balanced using iterative correction and eigendecomposition (ICE)³⁰, which normalizes all rows and columns of a contact matrix sum to the same value. Applying ICE balancing to all mapped reads generated subpar normalization and generated an artifact where ‘stripes’ containing no capture probe coverage appeared to have greater contact densities than adjacent probe-covered regions (FIG. 6E). ICE balancing to .mcool files containing data only within captured regions of interest (ROIs) did not result in these artifacts, and was therefore used in for all RCMC data in this study. The success of ICE balancing applied to these ROI-only .mcools was evaluated against published whole-genome Hi-C³¹ and Micro-C¹² data-sets in mESCs (FIG. 11A). The sum of each row of each of the RCMC, Micro-C, and Hi-C balanced contact matrices at 250-bp resolution within capture ROIs was calculated, plotted as a histogram distribution of row sums and verified to match the distribution of column sums. The subset of RCMC rows containing microcompartment anchors was also plotted to confirm that they match the distribution of row sums across the whole locus, ruling out that microcompartments are an artifact of incomplete ICE normalization³⁰.

### Visualizing RCMC.

RCMC contact maps were visualized alongside genomic annotations, published ChIP-seq, RNA-seq, and ATAC-seq datasets using the HiGlass⁵⁴ browser (http://higlass.io/) and software (v0.8.0). Contact maps shown in figures were generated using cooltools (v0.5.0) (https://cooltools.readthedocs.io/). Genomic tracks (that is, ChIP-seq, RNA-seq, and ATAC-seq) and gene annotations in figures were generated using CoolBox⁵⁵ (v0.3.3). In generating the genomic tracks, 27 public datasets were analyzed (Table 18) using processed bigWig files that were CrossMapped⁵⁶ (v0.6.1) (http://crossmap. sourceforge.net/) to the mm39 reference genome. Tracks were visualized using the Integrative Genomics Viewer⁵⁷ (v2.10.3) to scale tracks by identifying local maxima and minimizing noise.

### Supplementary Protocol

### Experimental Procedure

### Nuclear Extract and Immunoblotting

To confirm depletion of RAD21-mAID, nuclear extracts were prepared from cell pellets harvested in parallel to the preparation of cells for RCMC. Cell pellets were washed with PBS and stored at −80° C. prior to extraction. Cell pellets were thawed on ice and resuspended in 10 volumes of buffer A (10 mM HEPES pH 7.9, 1.5 mM MgCl₂, 10 mM KCl, 0.5 mM DTT, 0.5 mM PMSF, and Complete protease inhibitor (PIC, Roche #11697498001)). Cells were incubated for 10 min on ice before centrifugation at 1,500×g for 5 min at 4° C. Cell pellets were then resuspended in 3 volumes of buffer A containing 0.1% IGEPAL CA-630 (Sigma-Aldrich #18896) and inverted to mix. Nuclei were pelleted again at 1,500×g for 5 min at 4° C. Nuclei were resuspended in 1 volume of buffer C (250 mM NaCl, 5 mM HEPES pH 7.9, 26% glycerol, 1.5 mM MgCl₂, 0.2 mM EDTA, 0.5 mM DTT and 1×PIC), the volume of the suspension measured, and NaCl concentration adjusted to 400 mM. After 1 h of incubation on ice, nuclear proteins were recovered as the supernatant by centrifugation at 18,000×g for 20 min. Protein concentration was determined using the Qubit Protein BR Assay (ThermoFisher Scientific #A50668).

Nuclear extracts were prepared for loading by mixing with SDS loading buffer and boiling at 95° C. for 5 min. Proteins were resolved by SDS-polyacrylamide gel electrophoresis (SDS-PAGE), using 4-15% Mini-PROTEAN TGX gels (Bio-Rad #4561084) at 200 V for 45 min in 1×Tris/Glycine/SDS buffer (Bio-Rad #1610732). Proteins were transferred to PVDF membrane by wet transfer using the eBlot L1 system (GenScript #L00686, Standard pre-programmed transfer settings). Membranes were blocked using 5% milk in 1× PBS with 0.1% Tween 20 (PBST) for 30 min at room temperature. Membranes were then incubated overnight at 4° C. in the same buffer with primary antibodies (α-RAD21, Abcam #ab154769, 1:1,000; α-TBP, Abcam #ab818, 1:3,000). Membranes were washed for 3×5 min with PBST before incubation with HRP-conjugated secondary antibody (Rabbit IgG HRP Linked Whole Ab, Millipore Sigma #GENA934, 1:5,000; Mouse IgG HRP Linked Whole Ab, Millipore Sigma #GENA931, 1:5,000) for 1 h in 5% milk in PBST. Following secondary antibody incubation, membranes were washed for 3×5 min in PBST and rinsed with PBS prior to incubation with Clarity Max Western ECL Substrate (Bio-Rad #1705060) for 1 min. Membranes were then imaged using the ChemiDoc Imaging System (BioRad #17001401).

### Calibrated RNA Polymerase II ChIP-Seq

To confirm successful triptolide inhibition of RNA Pol II, spike-in calibrated RNA Pol II ChIP-seq was performed on cells harvested in parallel to preparation of treated cells for RCMC. For each condition, 5×10⁷ cells were suspended in 10 mL of PBS and crosslinked in 1% formaldehyde for 10 min at room temperature, followed by quenching with 125 mM glycine. For whole-cell spike-in, HEK293T cells were prepared in the same way in batches of 2×10⁶ cells. Following fixation, cells were washed with PBS, snap-frozen in liquid nitrogen, and stored at −80° C. To prepare chromatin, cells were thawed on ice, and each sample containing 5×10⁷ mESCs was mixed with 2×10⁶ HEK293T cells and resuspended in a small volume of ice-cold PBS. The cell mixture was then resuspended entirely in 750 μL of FA-lysis buffer (50 mM HEPES, pH 7.9, 150 mM NaCl, 2 mM EDTA, 0.5 mM EGTA, 0.5% NP-40, 0.1% sodium deoxycholate, 0.1% SDS, 10 mM NaF, 1 mM AEBSF, 1× PIC) and incubated on ice for 10 min. The suspension was sonicated using an E220evolution focused-ultrasonicator (Covaris; peak incident power: 140 W, duty factor 5%, cycles per burst 200, temperature 4° C., time 12.5 min) in milliTUBE 1 mL with AFA Fiber (Covaris #520135). Chromatin was taken as the supernatant following centrifugation of the sonicated samples at 20,000×g for 10 min at 4° C.

For each chromatin immunoprecipitation reaction, 300 μg of chromatin was diluted to 1 mL with FA-lysis buffer and then precleared for 1 h at 4° C. with protein A magnetic Dynabeads (Invitrogen #10001D) blocked with 1 mg/mL BSA and 1 mg/mL yeast tRNA. Diluted and pre-cleared chromatin was incubated overnight at 4° C. with antibody (α-RPB1 NTD, Cell Signaling Technology #14958, 10 μL per ChIP). Antibody-bound chromatin was pulled down using blocked protein A Dynabeads for 3 h at 4° C. Bead were then washed with FA-lysis buffer, FA-lysis buffer with 500 mM NaCl, DOC buffer (10 mM Tris-HCl pH 8, 250 mM LiCl, 2 mM EDTA, 0.5% IGEPAL CA-630, 0.5% Na-deoxycholate), and twice with TE prior to elution in elution buffer (1% SDS, 0.1 M NaHCO₃) by incubation for 30 min at 30° C. Eluted chromatin was de-crosslinked at 65° C. overnight with 200 mM NaCl and 2 μL RNase A. Input equivalent to 5% of the original ChIP reaction was treated identically. Reverse crosslinked samples were incubated with proteinase K for 1 h at 45° C., and then purified using the ChIP DNA Clean and Concentrator kit (Zymo Research #D5205).

Libraries for ChIP-seq were prepared for all samples using the NEBNext Ultra II DNA Library Prep kit for Illumina (NEB #E7645) according to the manufacturer's protocol, and samples were individually indexed using NEBNext Multiplex Oligos for Illumina. Library concentration and fragment size were assessed by qPCR and Fragment Analyzer (Agilent).

### Data Analysis

### Comparing Data Across Methods

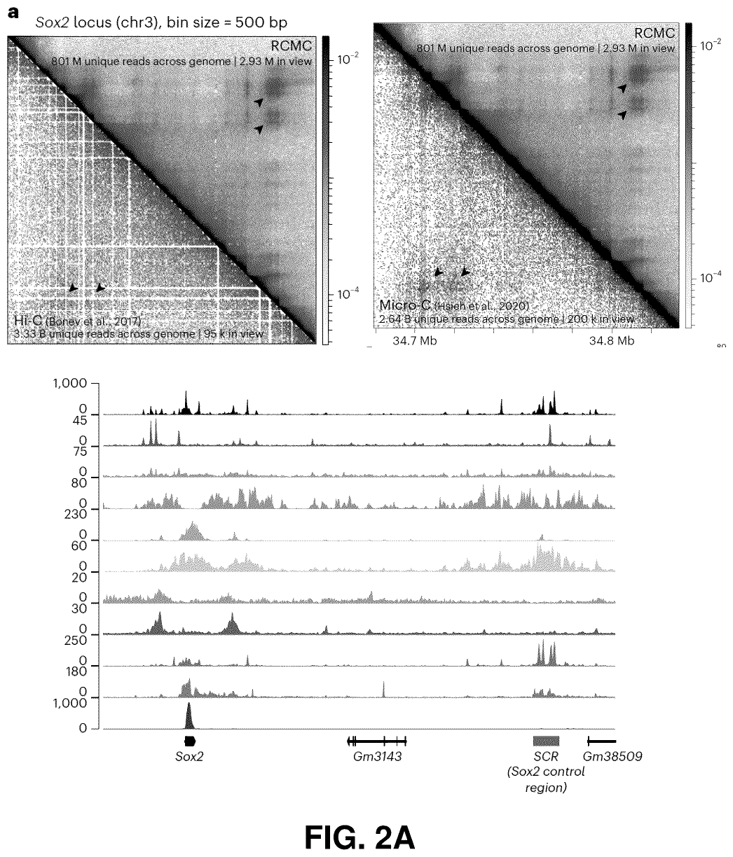

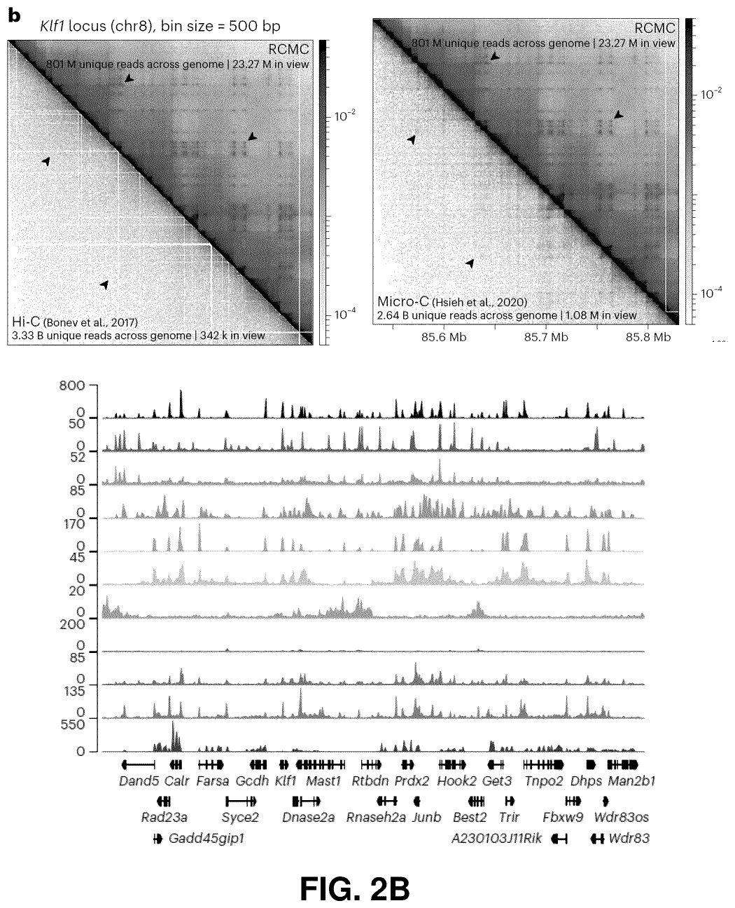

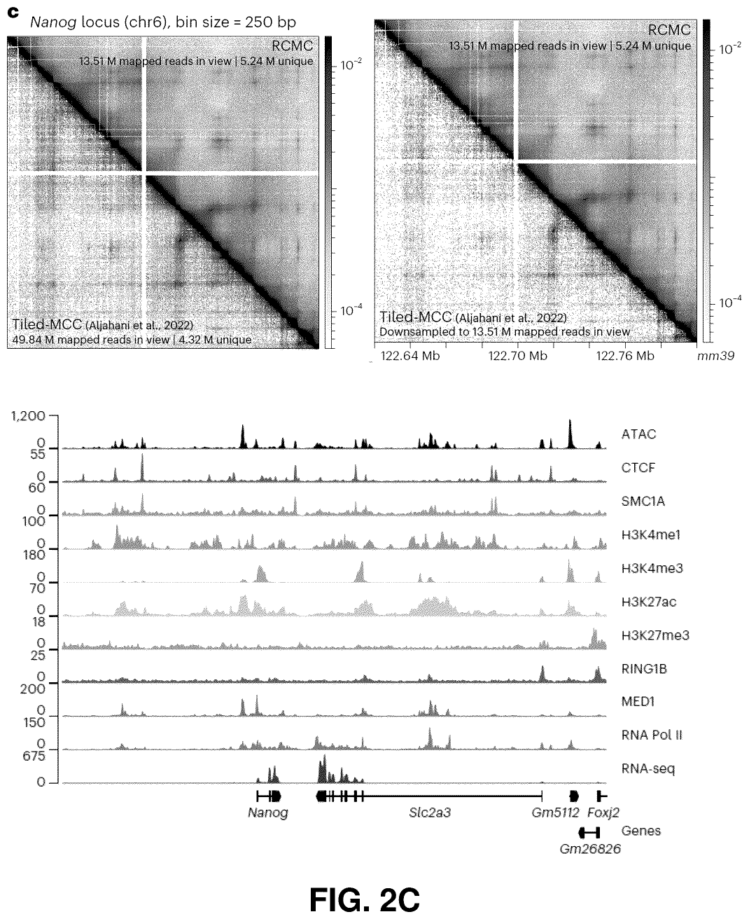

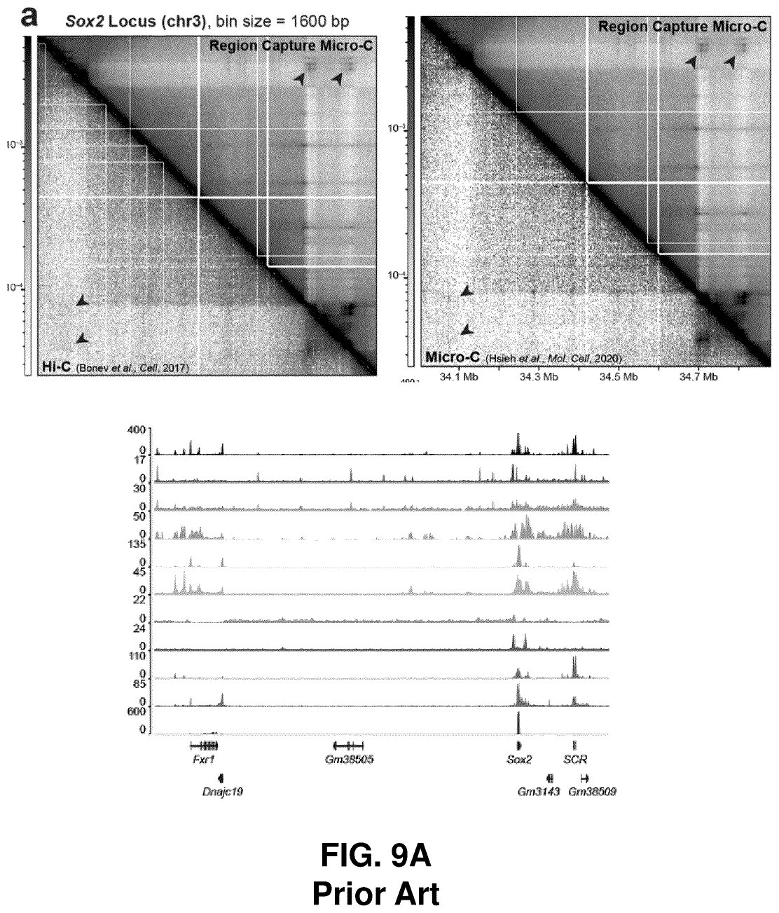

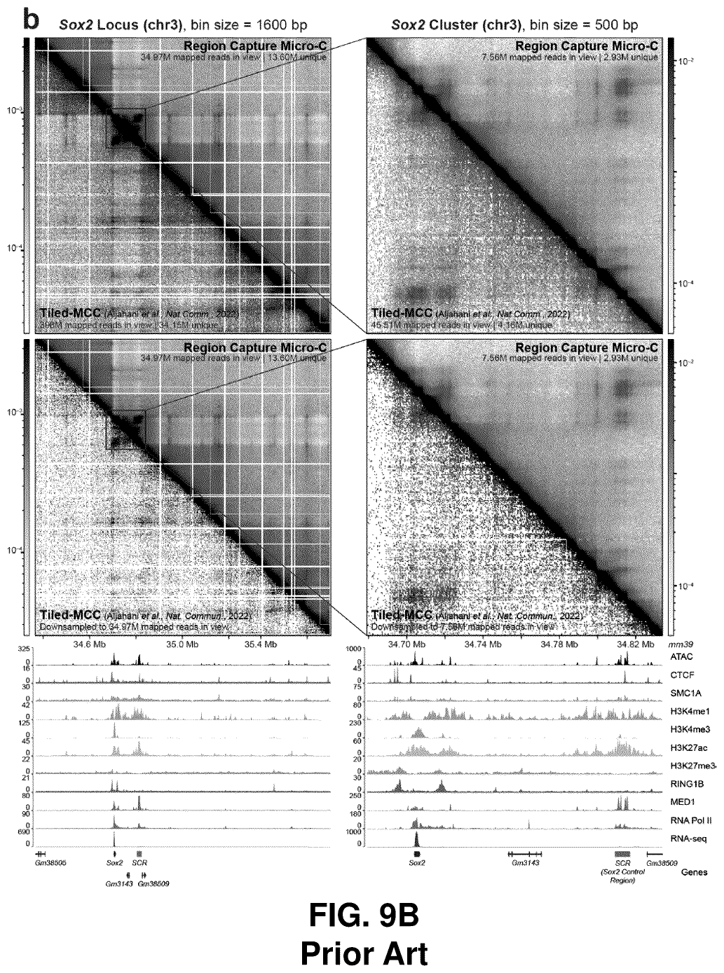

Mapped sequencing reads were filtered using pairtools select to quantify read counts according to chosen evaluation criteria (FIGS. 1B, 6D, 7C). Filtering was performed identically across the RCMC, TMCC, Micro-C, and Hi-C datasets on .pairs files containing mm39-mapped reads. RCMC .pairs files were generated as described above, while mm10-aligned .pairs files containing all unique reads were downloaded for Hi-C (GSE96107) and Micro-C (GSE130275) and CrossMapped to the mm39 genome. TMCC .pairs files were generated through two methods. For TMCC contact map generation and all analyses using unique contacts (FIGS. 1B, 2C, 7A-C, 9B), curated lists of unique contacts (in mm10) were downloaded for wild-type TMCC data (GSE181694), converted to a .pairs data format, and CrossMapped to the mm39 genome. For quantifications of total mapped reads (FIG. 7C, downsampling in FIGS. 2C, 9B), raw sequencing data files were downloaded for TMCC and aligned to the mm39 genome in a similar manner to RCMC.

Quantifications of read coverage across bins (FIGS. 1B, 7B) were calculated in Python using cooler to load unbalanced 100 bp resolution .cool files into memory as matrices. These matrices were then iterated through to determine the fractions of bins containing at least one read at different contact distances.

Genome-wide equivalents for RCMC data were calculated by extrapolating the number of unique contacts mapped to a Capture locus to a region the size of the entire mouse genome. This approach assumes homogeneous read coverage throughout the genome; in reality, however, read coverage is unevenly distributed between regions depending on the specific region and which 3C method is used. Specifically, both genome-wide Hi-C (3.3B total unique) and Micro-C (2.64B total unique) also had higher coverage at the Klf1 region than the genome-wide average. As such, compared to Hi-C at the Klf1 locus, RCMC captured ˜38-fold more unique contacts. Similarly, compared to genome-wide Micro-C5 at the Klf1 locus, RCMC captured ˜ 12-fold more unique contacts. Nevertheless, even with ˜100-fold downsampling of RCMC data at the Klf1 locus, the observed focal interactions remained more clearly visible in RCMC than in Micro-C (FIG. 7F).

### Contact Decaying Curve Analysis

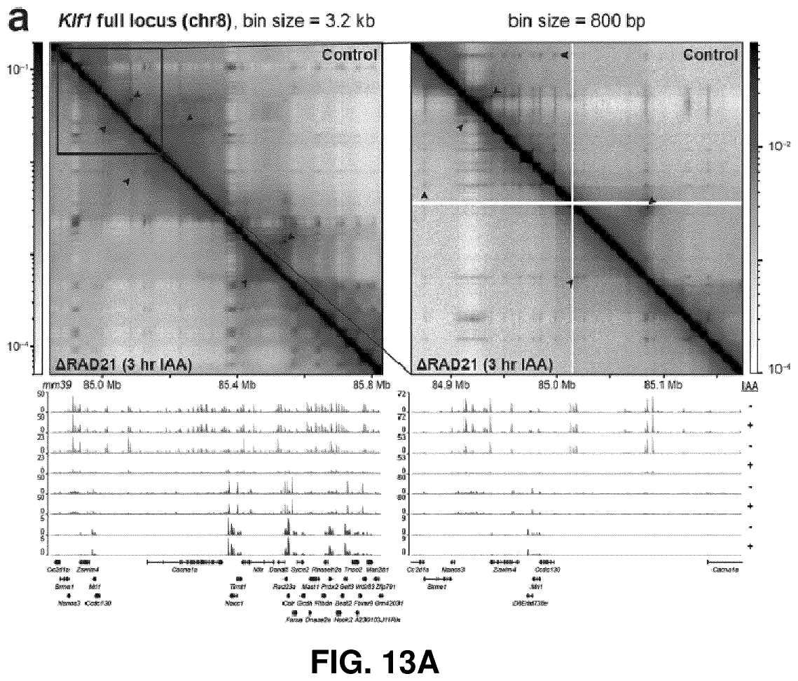

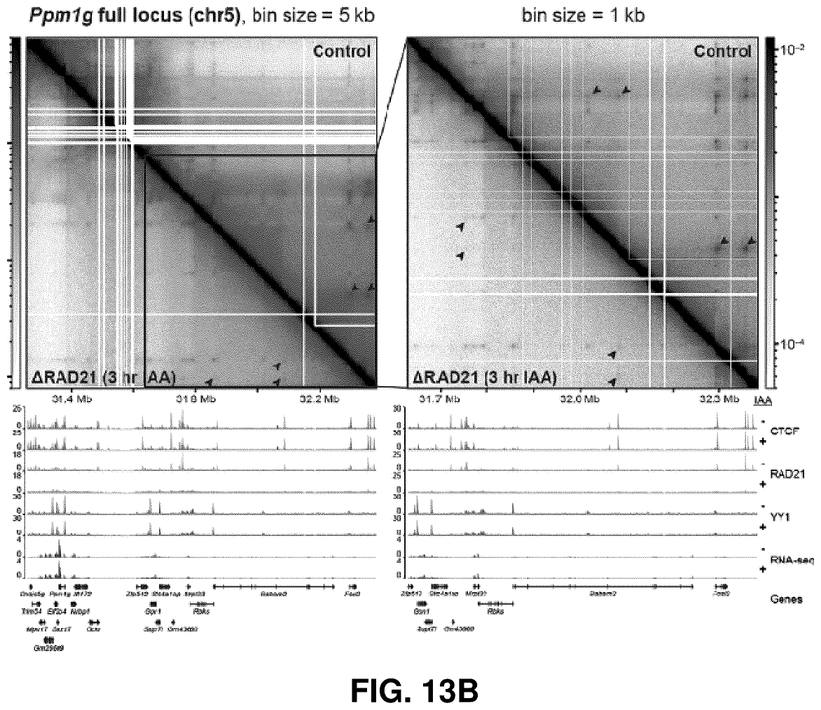

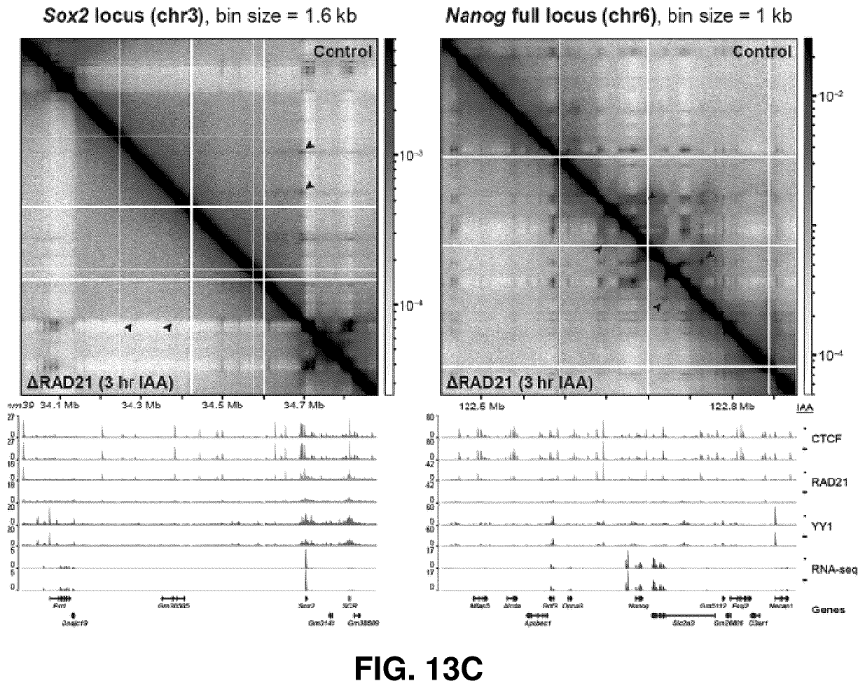

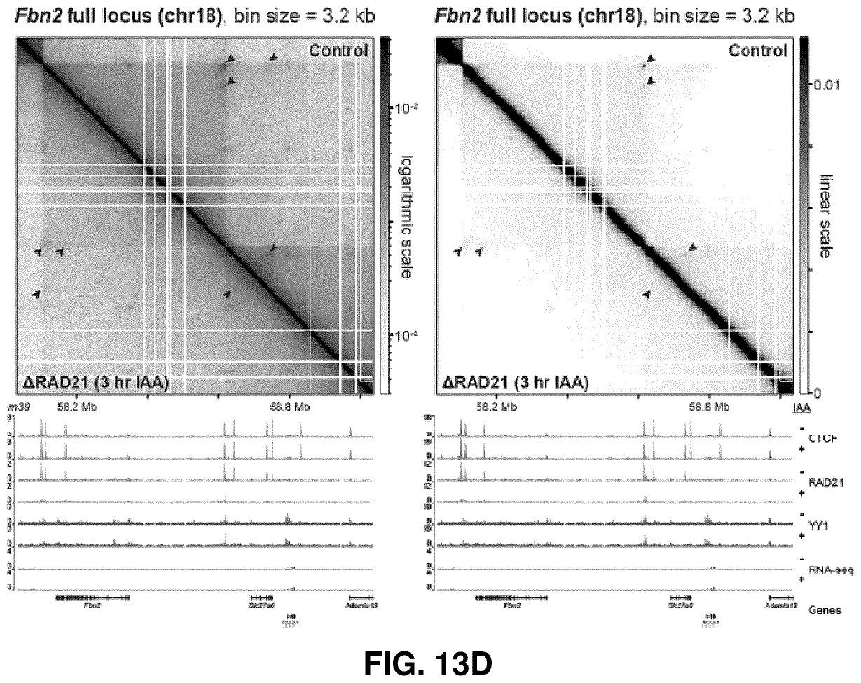

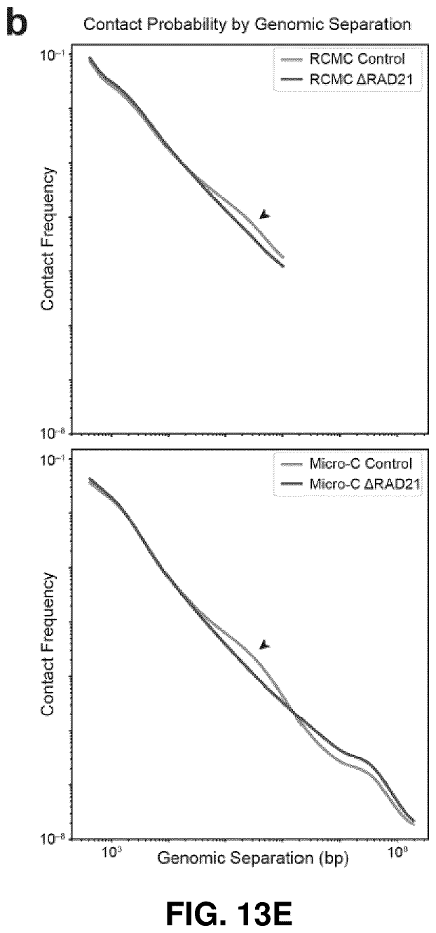

Contact decay curves were generated by plotting contact probability against genomic separation using cooltools (FIGS. 7A, 13B). Balanced and smoothed curves were generated using contact matrices across each chromosome binned to 50 bp resolution. RCMC and TMCC curves were truncated at 1 Mb genomic separation due to noise at larger genomic separations (the largest Captured locus for each method is between 1-2 Mb in size).

### Replicate Reproducibility Analysis

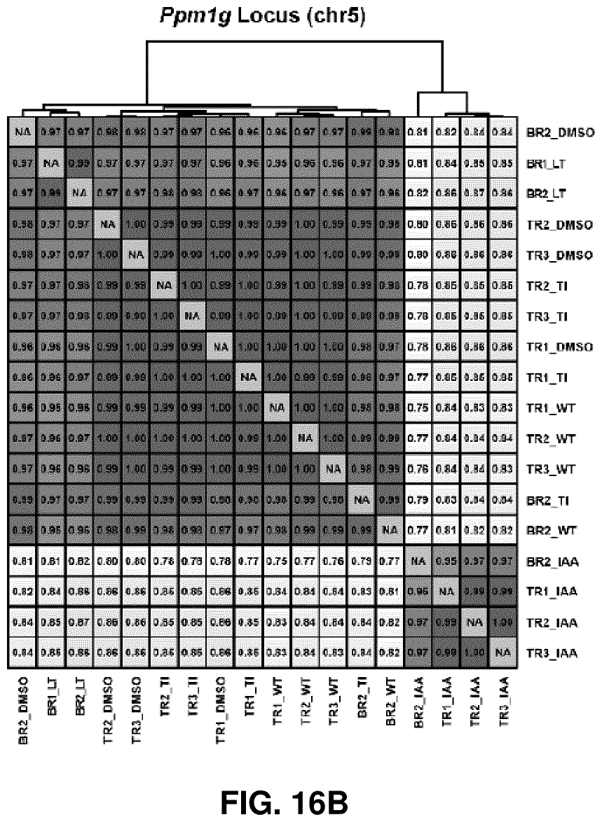

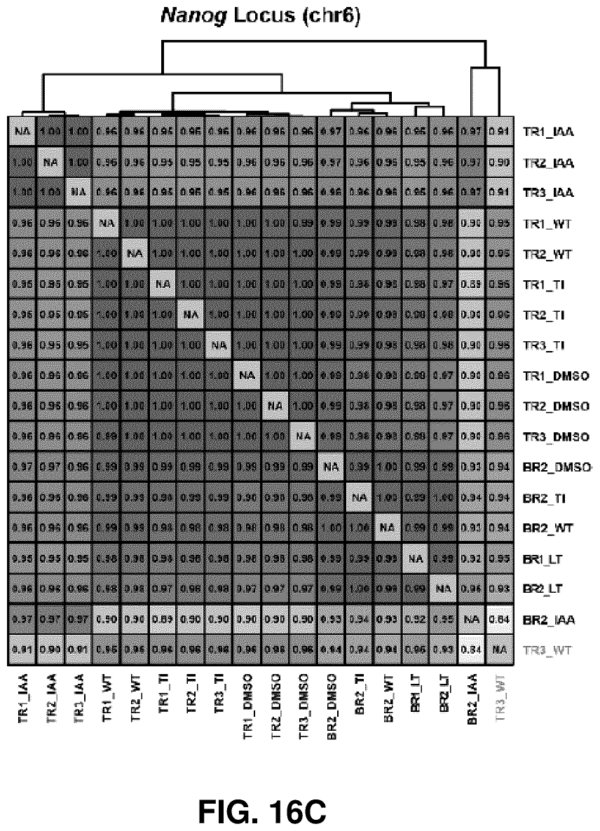

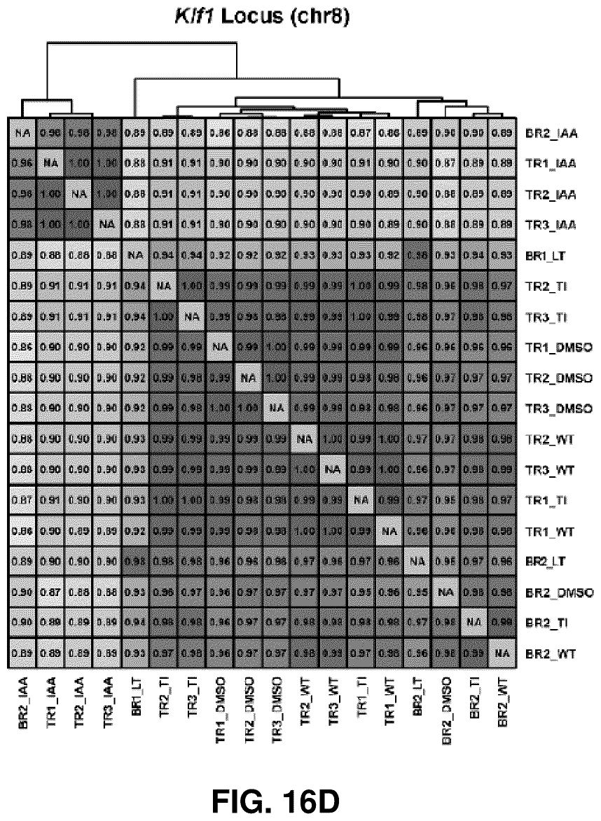

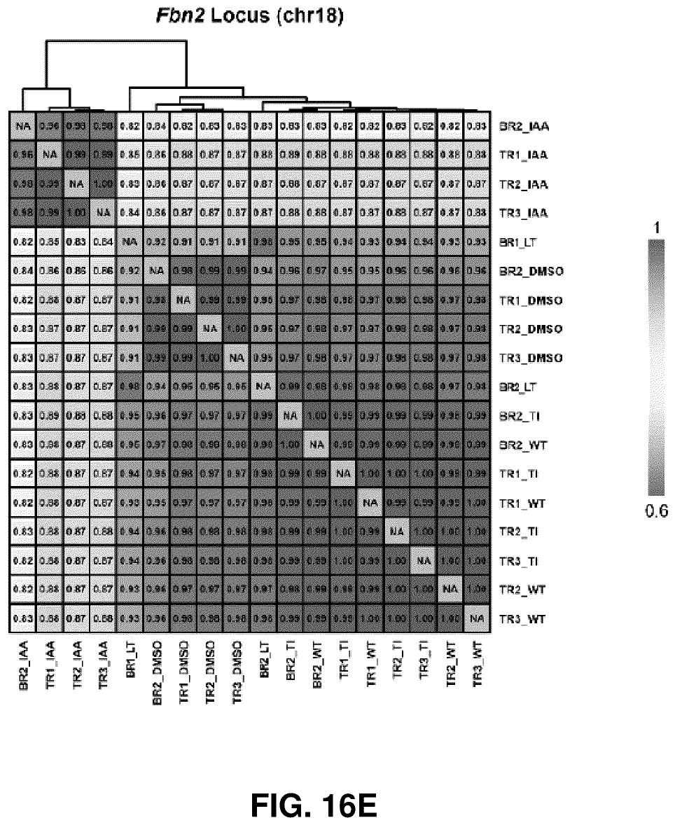

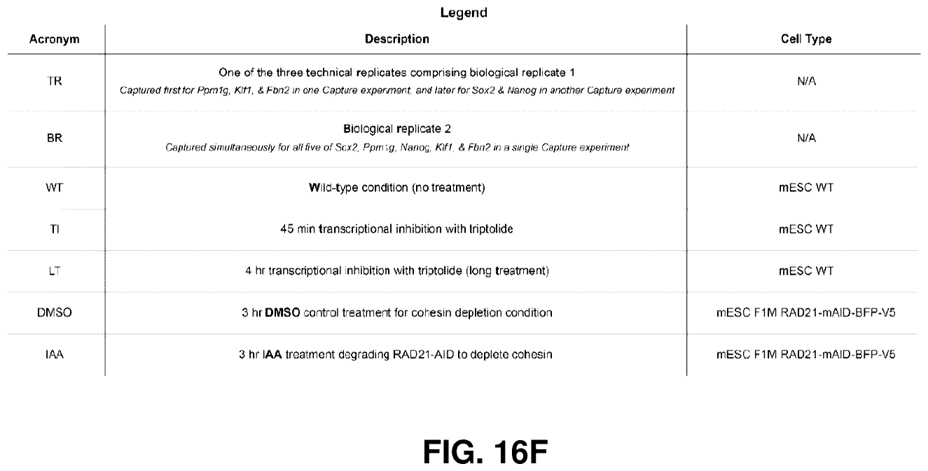

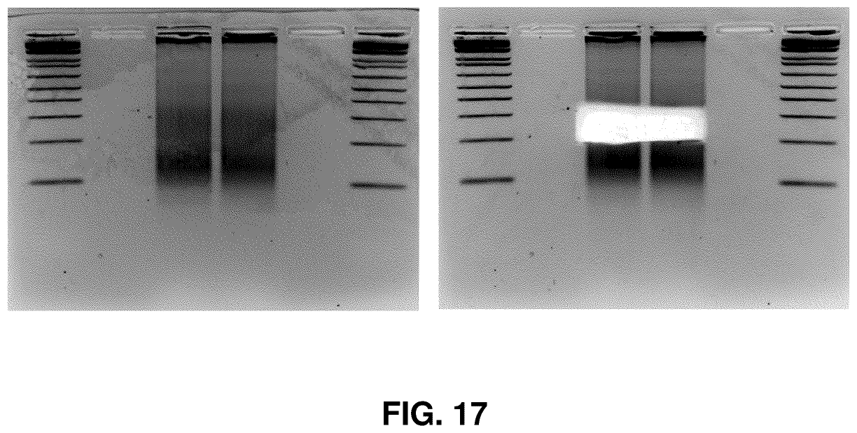

The reproducibility of RCMC technical replicates (FIGS. 6G, 16A-E) was evaluated using HiCRep (v1.12.2) for contact maps at 10 kb resolution, with parameters lbr=0 and ubr=5000000. Reproducibility scores were calculated for each region independently for all replicates using the optimal h-value determined from a single replicate.

### Downsampling

RCMC and Tiled-Micro-Capture-C (TMCC) were downsampled using pairtools sample to randomly select a subset of the mapped contact pairs. To downsample RCMC (FIGS. 7D-F), a .pairs file containing all mapped reads across all replicates was downsampled using downsampling ratios corresponding to orders of two from 2 to 128. Each downsampled .pairs output file was then filtered for reads with both mates within one of the five Captured loci, the unique reads were extracted, and a .mcool was generated for visualization purposes.

To ensure parity in comparison across methods when downsampling TMCC, .pairs files containing all mapped reads (all unique and duplicate reads with both mates in the viewpoint) were first generated for viewpoints of interest in the Sox2 (FIG. 9B) and Nanog (FIG. 2C) loci. These files yielded the total number of mapped reads necessary to generate the density of information present in the viewpoint, and a downsampling ratio was calculated to match the total number of mapped TMCC reads in the viewpoint with RCMC. After a randomly downsampled TMCC .pairs file was generated, the unique reads were extracted and used to generate a .mcool for visualization purposes.

### Chromatin Contact Analysis

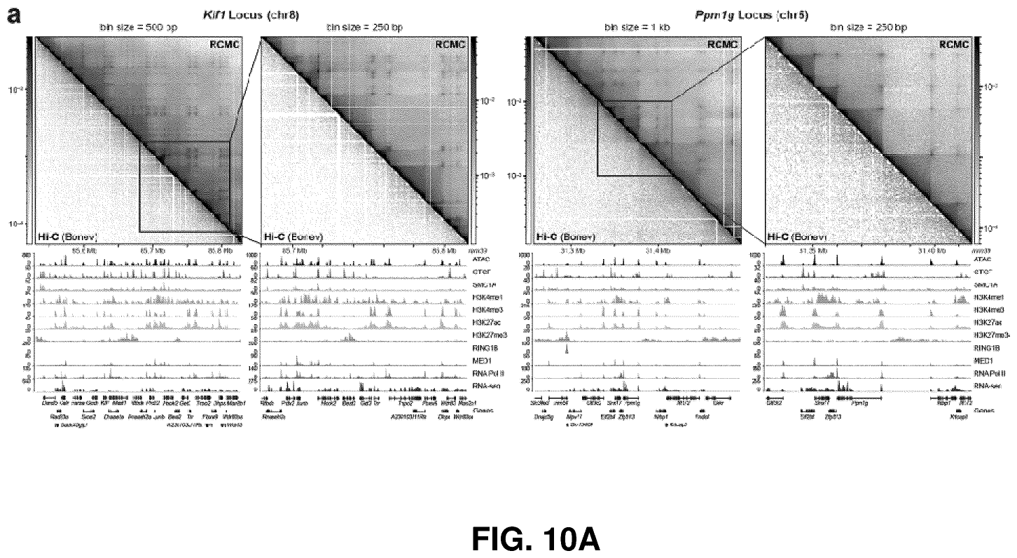

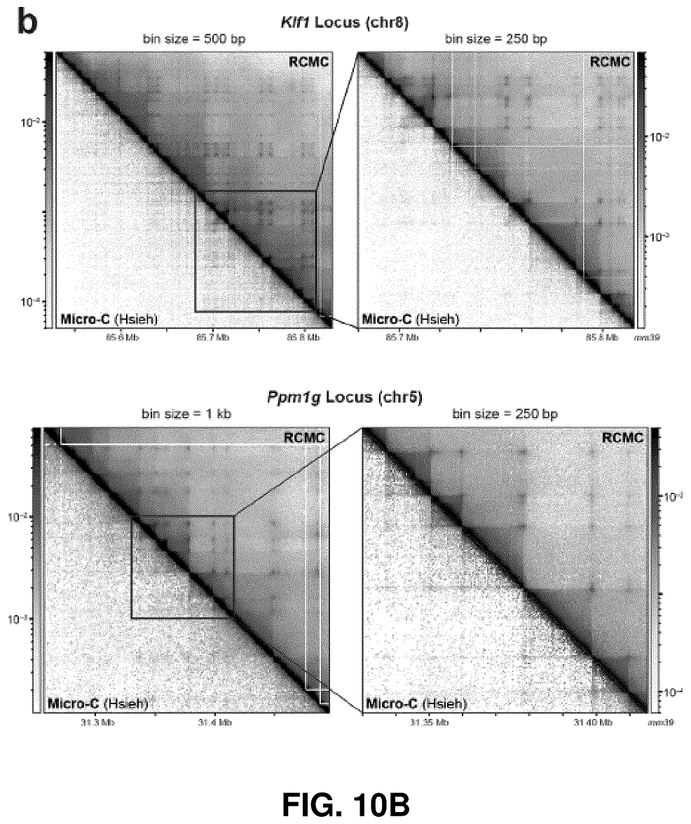

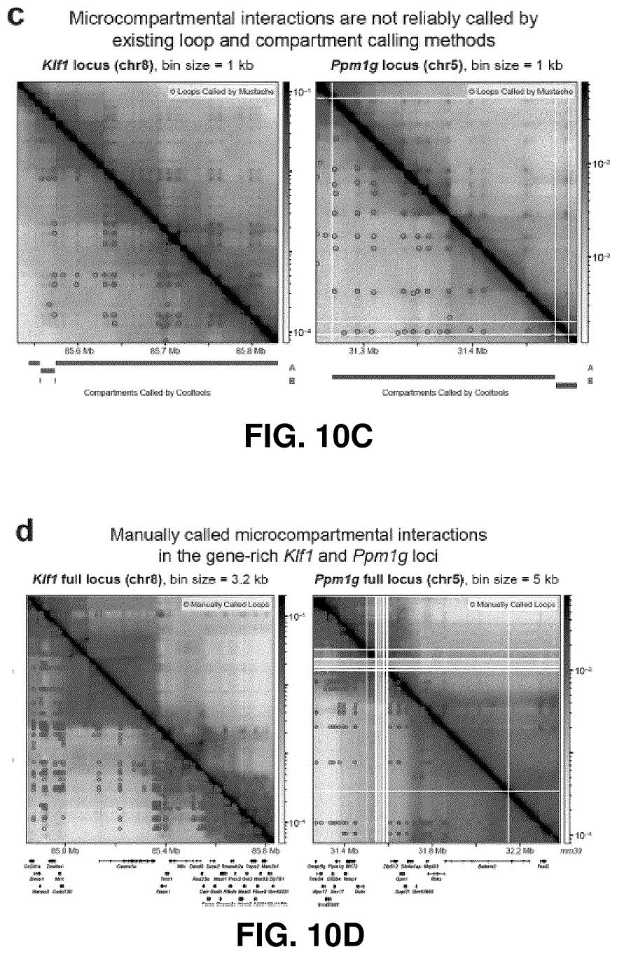

Chromatin loops were called on WT Capture ROI-only RCMC data using Mustache (v1.2.4) (github.com/ay-lab/mustache) at 0.25, 0.5, 1, 2, 5, and 10 kb data resolutions with sparsity thresholds of 0.7 and q-value thresholds of 0.1 (FIG. 10C). Loop-calling was also tested using Chromosight (v1.6.1) (github.com/koszullab/chromosight) and SIP (v1.6.1) (github.com/PouletAxel/SIP), with Mustache producing similar or greater numbers of loop calls. Finer resolutions of loop calling called more microcompartmental contacts, but still missed some contacts while also increasingly misidentifying stripes as loops, overlapping loops, and clustering calls at short contact distances off of the diagonal.

Manual contact-calling was performed in an attempt to minimize these artifacts in microcompartment analysis. Contacts were defined as punctate foci of interaction (i.e., “dots”), visibly discernible as being enriched relative to their local background. Diffuse and overly faint interactions, homogeneously enriched stripes, and short-range contacts just off of the diagonal (i.e., under ˜5 kb contact distance) were not identoiified as contacts. Contacts were called on ICE-balanced WT Capture ROI-only RCMC data at 250 bp resolution across the full Klf1 and Ppm1g loci using the HiGlass browser interface. Scale bar limits were dynamically modulated to minimize background and clearly distinguish focal enrichment. A total of 1091 focal contacts (loops) spanning 132 contact anchors were manually annotated across the two loci (FIG. 10D). Called contacts ranged in length from 4.6 to 1,018 kb between anchors, with a strong majority of contacts laying in the Klf1 locus.

### Compartmentalization Analysis

Compartments were called by applying eigendecomposition to the WT RCMC contact matrix using cooltools (FIG. 10C). Capture ROI-only RCMC data was first normalized to remove the distance-dependent effect of contact frequency. Eigendecomposition was then performed, with GC content serving as a correlate for orienting eigenvectors to indicate A-(gene-rich or active chromatin) or B-(gene-poor or inactive chromatin) compartments. Finally, eigenvectors were binarized and visualized as BED tracks using CoolBox. Compartment calling was performed at 0.5, 1, 5, and 50 kb data resolutions, with all producing similar output (calls at 1 kb resolution are shown for clarity's sake) or outright failing to call compartments.

### Microcompartment Anchor Classification

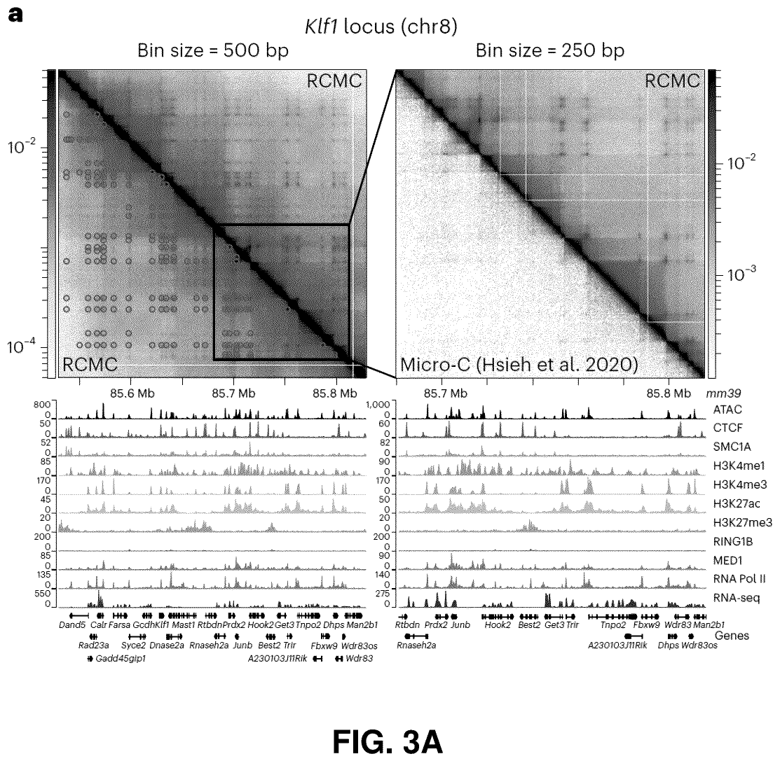

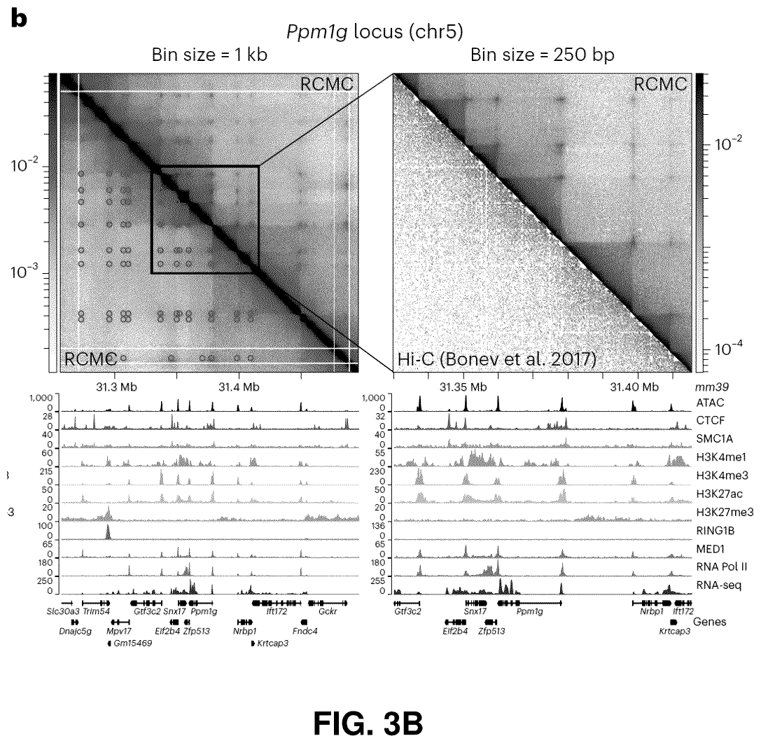

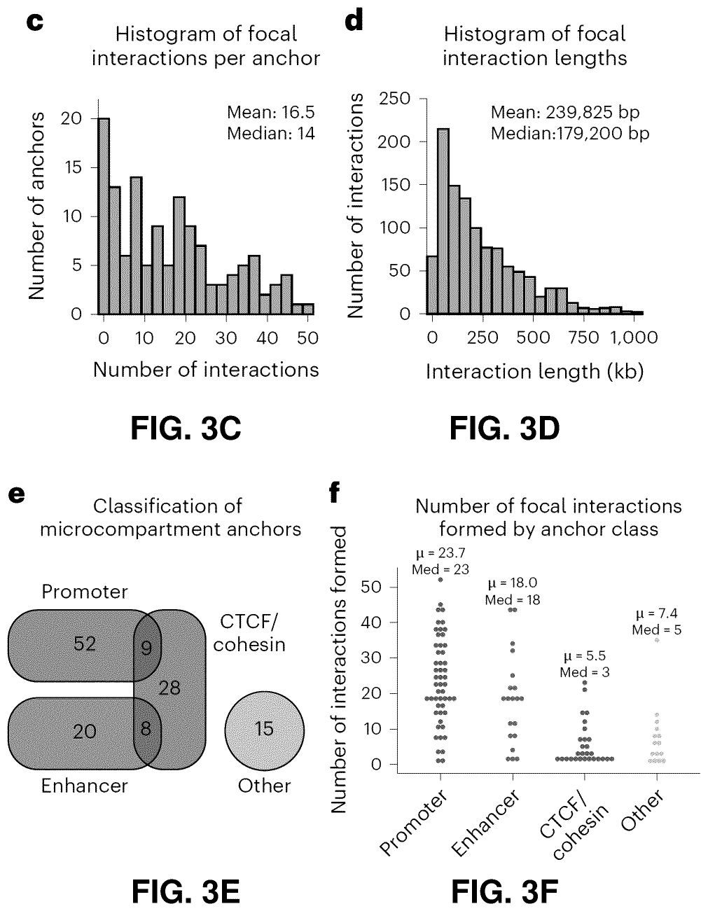

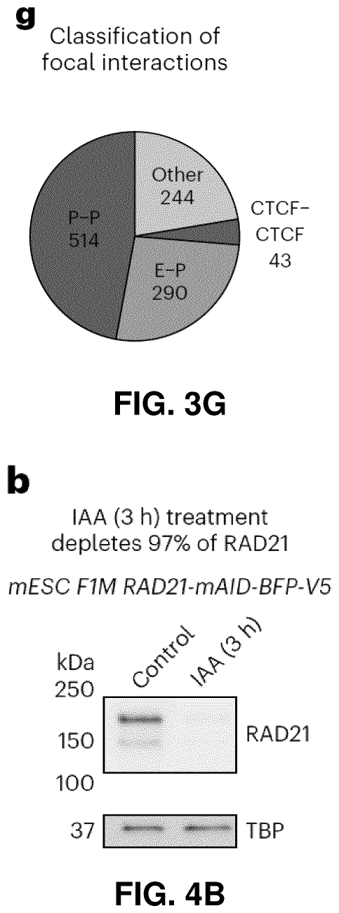

To classify microcompartments anchors as promoter, enhancer, or CTCF and cohesin-bound (FIG. 3E), microcompartment anchor locations were compared with the corresponding chromatin features as follows. Promoter regions were defined using all TSS locations in the mm39 UCSC RefGene annotation¹⁰±2 kb. Enhancers or CTCF and cohesin-bound sites were defined based on overlap of H3K4mel (ENCFF282RLA) and H3K27ac (GSE90893), or CTCF (GSE90994) and SMCIA (GSE123636), respectively. For all datasets, bigwig files were converted to bedgraph files using UCSC bigWigToBedGraph (v377)¹¹, followed by peak calling using MACS2 bdgpeakcall¹² (v2.2.7.1). For CTCF, called peaks were then overlapped with CTCF sites identified using FIMO (v5.4.1): first, fasta-get-markov was used to generate a background model using the mm39 genome assembly, then motifs were identified using-max-stored-scores 50000000-thresh 1e-3. Finally, locations of motifs were overlapped with peaks identified in ChIP-seq data, and only the motif with the highest score for each peak was maintained. The peaks (H3K4me1, H3K27ac, SMC1A) or identified sites (CTCF) were then overlapped using bedtools intersect (v2.30.0) to give enhancers or CTCF and cohesin-bound regions. Anchors of microcompartments ±1 kb were then overlapped with each of the three features to classify them as promoter, enhancer, or CTCF and cohesin-bound. Regions which were classified as both promoter and enhancer regions were treated as promoter-only due to the inability to distinguish H3K4mel/H3K27ac overlap at enhancers and promoters. Anchors overlapping none of these three features were classified as “Other”. Anchor classifications were then considered combinatorially to classify microcompartment interactions between regions (FIG. 3G).

Anchor overlap with ATAC-seq (GSE98390) peaks and vice versa was determined in the same way as for ChIP-seq data, after limiting ATAC-seq peaks to only those falling within the captured Klf7 and Ppm1g regions, to identify sets of regions which did or did not overlap (FIG. 11D).

The number of interactions formed by each anchor and the lengths of the interactions they form (FIGS. 3C, 3D, 3F) was determined and visualized in R¹³ (v4.1.2) using the GenomicRanges¹⁴ (v1.46.1) and ggplot2¹⁵ (v3.3.6) packages.

### Heatmap and Metaplot Generation

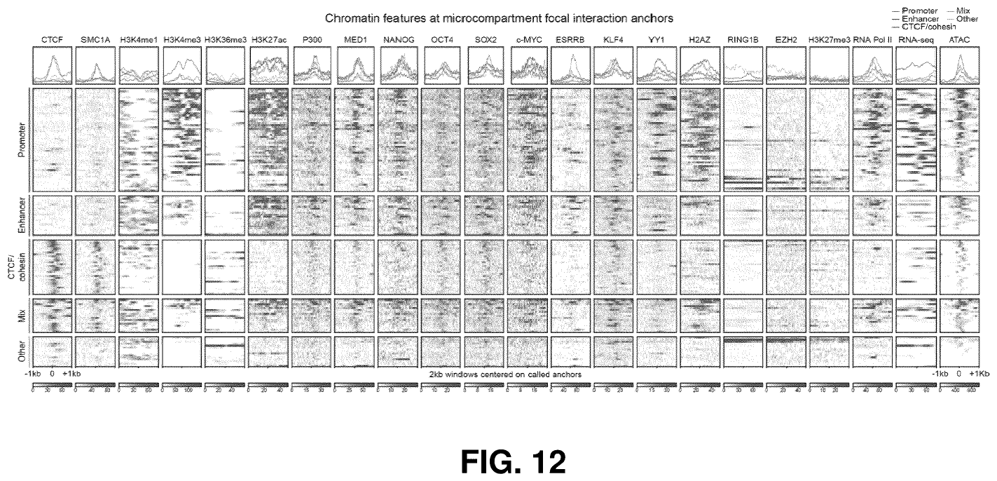

Heatmaps and metaplots were generated for microcompartments and ATAC-seq peaks using deeptools¹⁶ (v3.5.1) compute matrix followed by plot heatmap in a region ±1 kb around the center of each site for genomics data listed in Table 18 (FIGS. 11B, 12). Regions were sorted in all cases according to decreasing ATAC-seq signal. For RNA Pol II ChIP-seq, metaplots over gene bodies were generated using a list of unique genes derived from the RefSeq database¹⁷, with gene bodies scaled to 10 kb in length, leaving 500 bp at the 5′ end of the gene body unscaled, and including an unscaled region of ±3 kb before the TSS and after the TES, respectively.

### ChIP-Seq Data Analysis

Calibrated RNA Pol II ChIP-seq paired-end reads were aligned to a concatenated genome containing mouse (mm39) and human (hg38) genomes using bowtie2 (‘-no-mixed’ and ‘-no-discordant’ options). Only reads which aligned uniquely were retained for downstream analysis. PCR duplicates were removed using Sambamba markdup. For visualization and metaplot analysis of sequencing data, reads for each condition were randomly subsampled using normalization factors based on hg38 spike-in. To account for variation in spike-in cell mixing between different samples, normalization factors were corrected using the ratio of human to mouse read counts in corresponding input samples. Subsampled datasets were used for all visualization and downstream analysis.

TABLE 18

| | | | GEO/ | | |
|---|---|---|---|---|---|
| Organism | Sample | Description | ENCODE# | FIGS. | Reference |
| mESC | ChIP, CTCF | Architectural protein, loop | GSE90994 | 2, 3, EX1, EX2, EX3, | 1 |
| WT | | extrusion factor | | EX4, EX5, EX7 | |
| | ChIP, SMC1A | Architectural protein, loop | GSE123636 | 2, 3, EX1, EX2, EX3, | 2 |
| | | extrusion factor | | EX4, EX5, EX7 | |
| | ChIP, H3K4me1 | Histone marker, enhancers | ENCFF282RLA | 2, 3, EX2, EX3, EX4, | 3 |
| | | | | EX5, EX7 | |
| | ChIP, H3K4me3 | Histone marker, active genes | ENCFF523UIR | 2, 3, EX2, EX3, EX4, | 4 |
| | | | | EX5, EX7 | |
| | ChIP, | Histone marker, gene bodies | ENCFF848LEV | EX7 | 5 |
| | H3K36me3 | | | | |
| | ChIP, H3K27ac | Histone marker, enhancers | GSE90893 | 2, 3, EX2, EX3, EX4, | 6 |
| | | | | EX5, EX7 | |
| | ChIP, P300 | Enhancer factor | GSE90893 | EX7 | 7 |
| | ChIP, MED1 | General transcription factor | GSE22562 | 2, 3, EX2, EX3, EX4, | 8 |
| | | | | EX5, EX7 | |
| | ChIP, NANOG | Pluripotency transcription factor | GSE71932 | EX7 | 9 |
| | ChIP, OCT4 | Pluripotency transcription factor | GSE90893 | EX7 | 10 |
| | ChIP, SOX2 | Pluripotency transcription factor | GSE90893 | EX7 | 11 |
| | ChIP, c-MYC | Pluripotency transcription factor | GSE90893 | EX7 | 12 |
| | ChIP, ESRRB | Enhancer factor | GSE90893 | EX7 | 13 |
| | ChIP, KLF4 | Pluripotency transcription factor | GSE90893 | EX7 | 14 |
| | ChIP, YY1 | Architectural protein, | GSE99518 | EX7 | 15 |
| | | transcription factor | | | |
| | ChIP, H2AZ | Variant of H2A; found at active | GSE51579 | EX7 | 16 |
| | | promoters | | | |
| | ChIP, RING1B | Repressive chromatin factor | GSE96107 | 2, 3, EX2, EX3, EX4, | 17 |
| | | | | EX5, EX7 | |
| | ChIP, EZH2 | Repressive chromatin factor | GSE85717 | EX7 | 18 |
| | ChIP, | Histone marker, repressive | GSE90893 | 2, 3, EX2, EX3, EX4, | 19 |
| | H3K27me3 | chromatin | | EX5, EX7 | |
| | ChIP, RNA | Transcription | GSE58019 | 2, 3, EX2, EX3, EX4, | 20 |
| | Pol II | | | EX5, EX7 | |
| | RNA-seq | Transcription | GSE123636 | 2, 3, EX2, EX3, EX4, | 21 |
| | | | | EX5, EX7 | |
| | ATAC | Chromatin accessibility assay | GSE98390 | 2, 3, EX2, EX3, EX4, | 22 |
| | | | | EX5, EX6, EX7 | |
| mESC | ChIP, CTCF | Architectural protein, loop | GSE178982 | 4, EX8 | 23 |
| RAD21- | | extrusion factor | | | |
| mAID | ChIP, RAD21 | Architectural protein, loop | GSE178982 | 4, EX8 | 24 |
| (+/−cohesin | | extrusion factor | | | |
| depletion) | ChIP, YY1 | Architectural protein, | GSE178982 | EX8 | 25 |
| | | transcription factor | | | |
| | RNA-seq | Transcription | GSE178982 | 4, EX8 | 26 |
| GM12878 | ChIP, CTCF | Architectural protein, loop | ENCFF364OXN | EX10 | 27 |
| WT | | extrusion factor | | | |
| | ChIP, H3K27ac | Histone marker, enhancers | ENCFF180LKW | EX10 | 28 |
| | RNA-seq | Transcription | ENCFF604VIC | EX10 | 29 |

### Pile-Up and Contact Strength Analysis

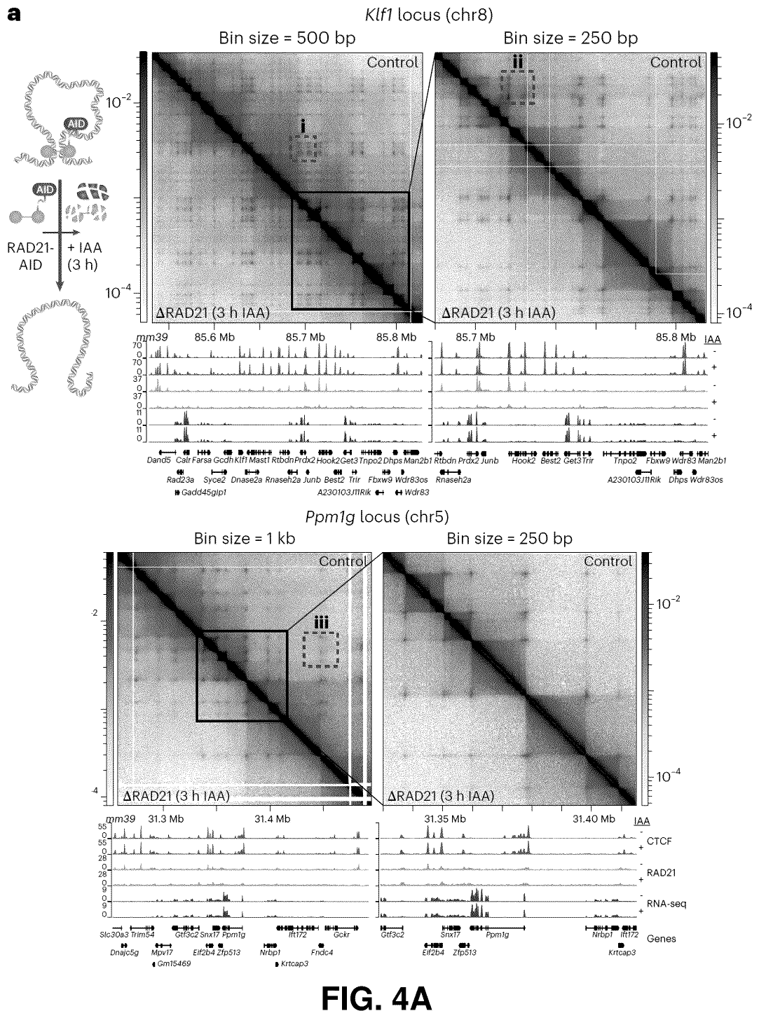

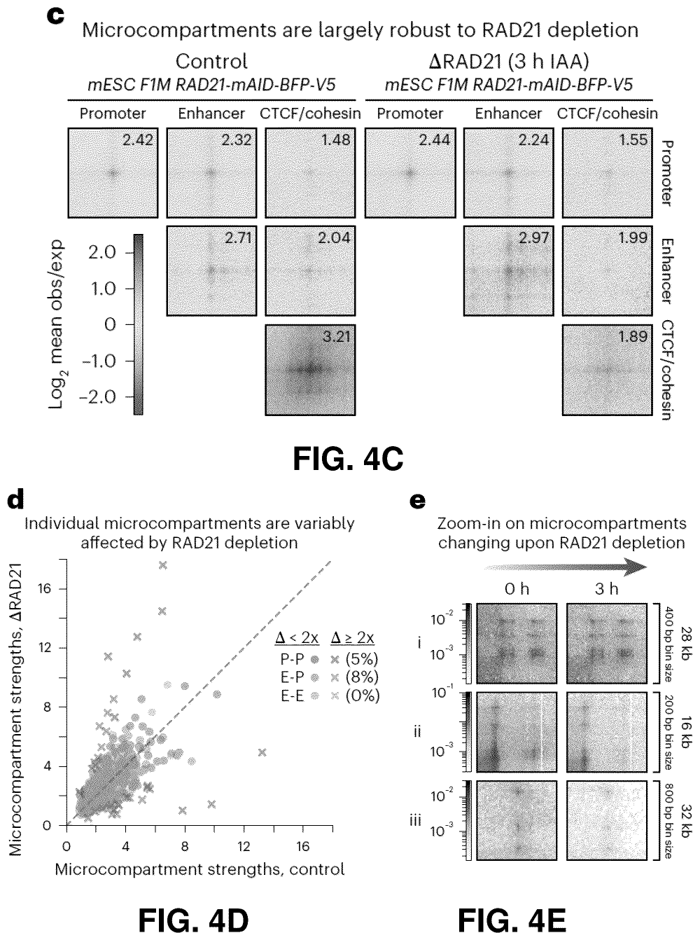

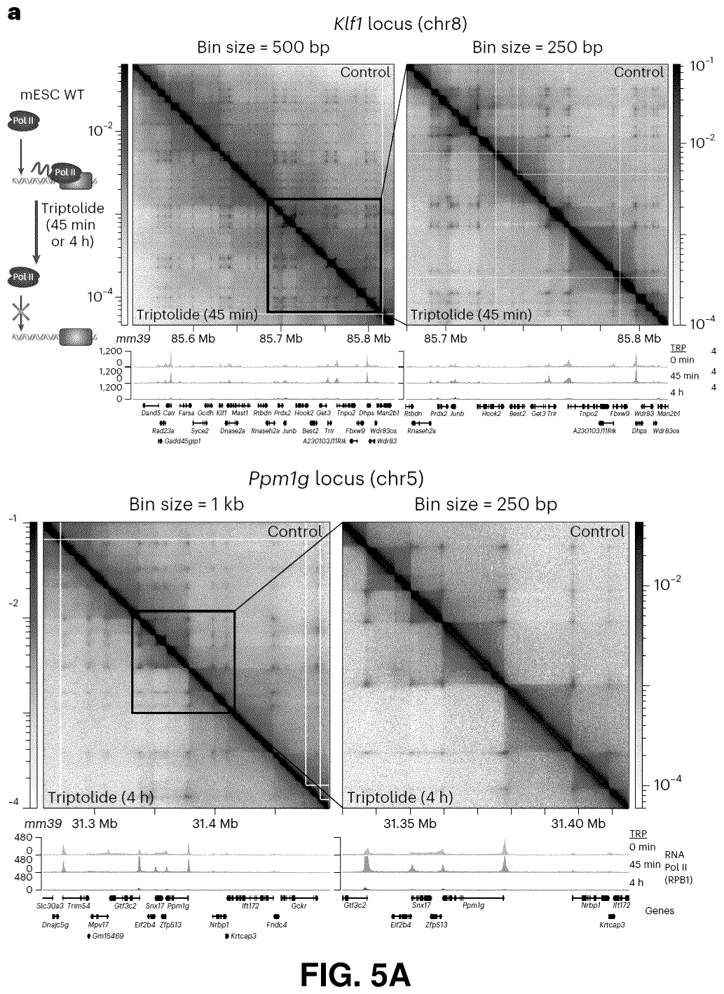

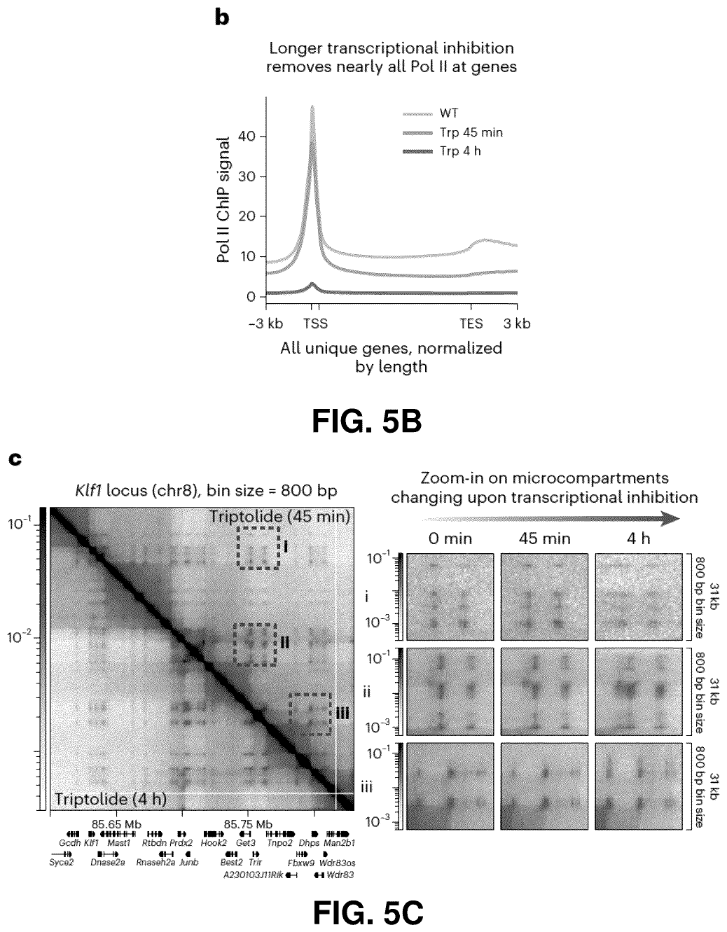

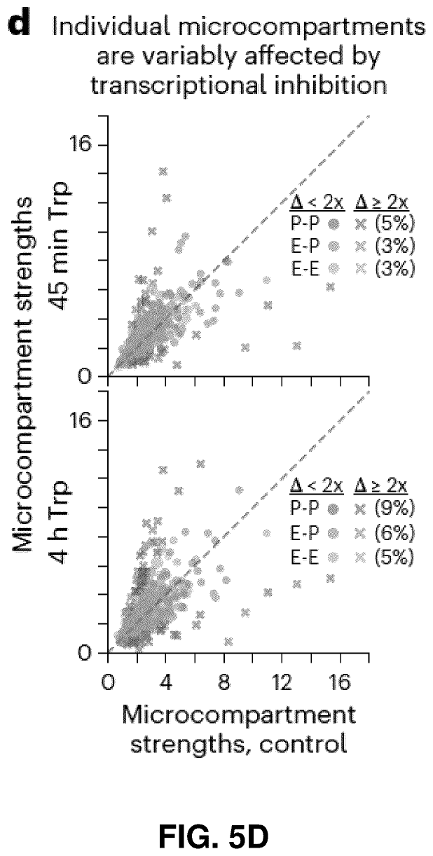

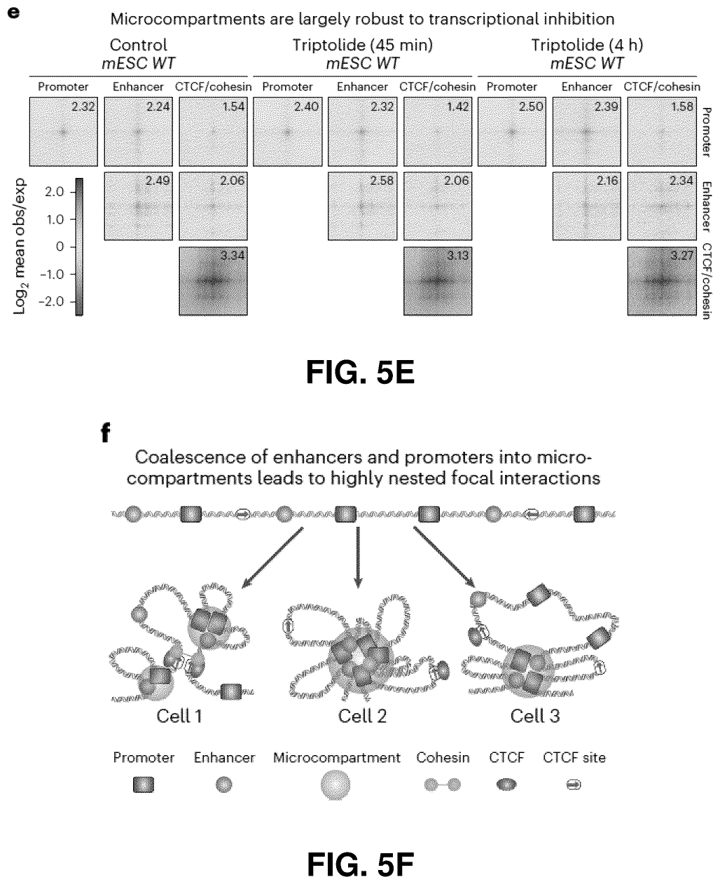

Pile-up visualizations and intensity quantifications of microcompartmental contacts were performed using cooltools to generate aggregate peak analyses (FIGS. 4C, 5E) and individual contact strength plots (FIGS. 4D, 5D). Plots for all contacts of a given classification (e.g., all WT Enhancer-Promoter microcompartments) were generated and analyzed individually or averaged for a 20 kb window centered on the contact at 250 bp resolution. Background-normalized dot enrichment values were calculated using 1250×1250 bp (5×5 pixel) boxes; the dot's average intensity values were determined by a box in the center of the viewing window whereas the background's average intensity values were determined by boxes in the top left and bottom right corners of the viewing window (chosen for their equivalent contact distances as the dot).

Visualizing Hi-C Data from Harris et al.

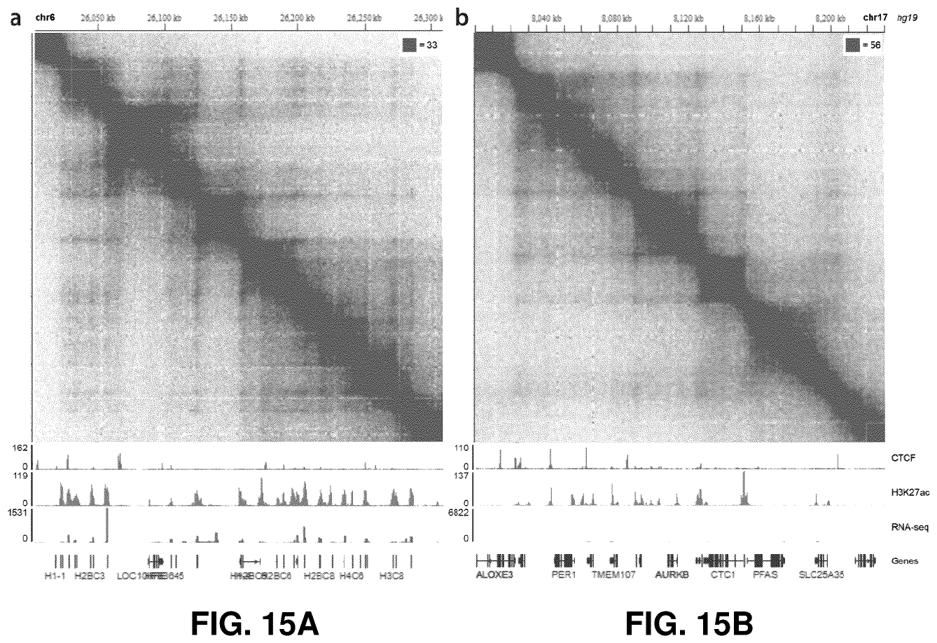

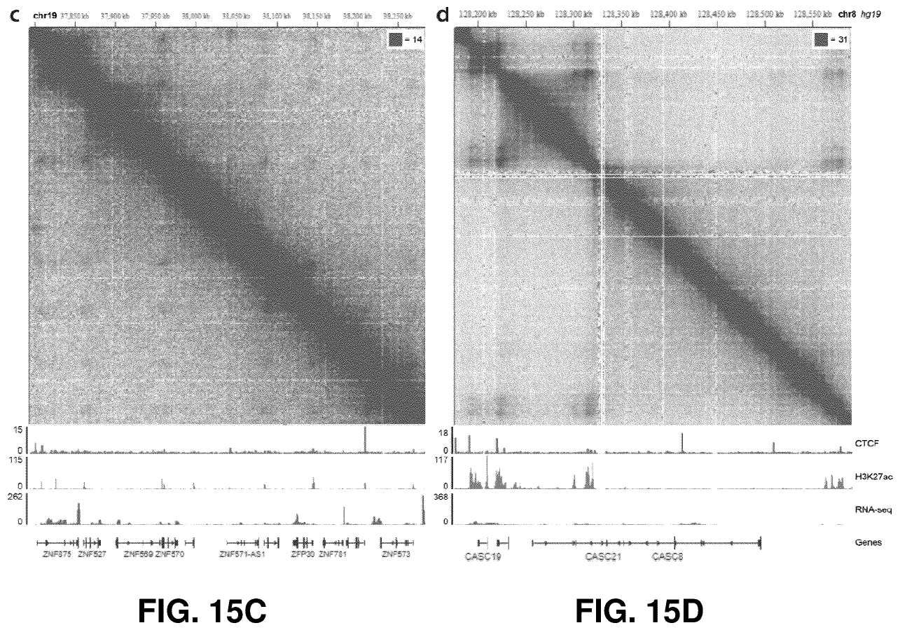

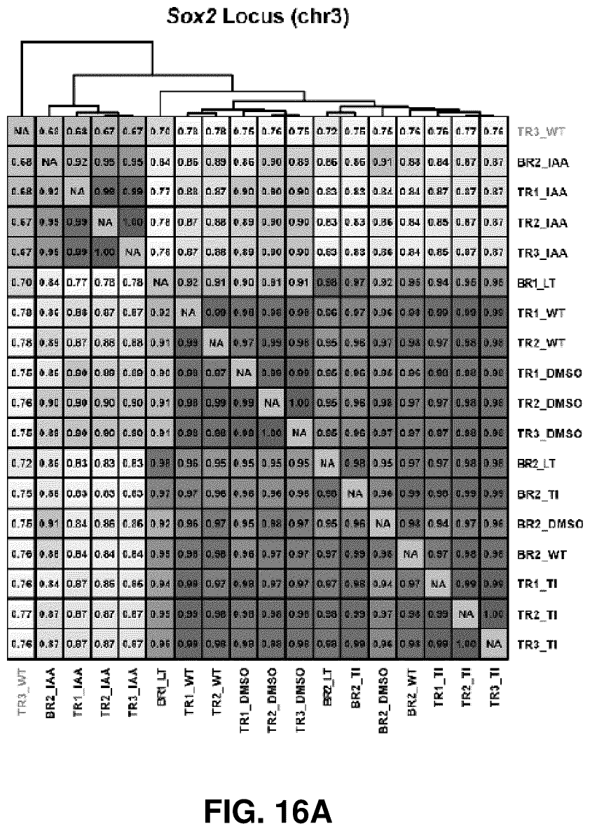

Hi-C contact maps generated in Harris et al. Nature Communications 2023 were visualized (hg19 reference genome) alongside genomic annotations, published ChIP-seq, and RNA-seq datasets in GM12878 cells using the Juicebox web interface (aidenlab.org/juicebox/). Screen captures were used to generate FIG. 15.

### Results

### RCMC: Development and Benchmarking

To develop Region Capture Micro-C(RCMC), the regular Micro-C protocol^{11,13,15} was improved using various design considerations, to maximize library complexity and combined the improved Micro-C with a tiling region capture approach^{28,29} (FIG. 1A). Briefly, mouse embryonic stem cells (mESCs) were crosslinked with disuccinimidyl glutarate (DSG) and formaldehyde (FA) and digested to nucleosomes with MNase (FIGS. 6A-B), after which fragment ends were repaired with biotin-labelled nucleotides and then proximity ligated. After protein removal and reversal of crosslinks, dinucleosomal fragments were-selected and ligated dinucleosomal fragments pulled down, and a Micro-C sequencing library was prepared. Avoiding repetitive regions, 80-mer biotinylated oligos tiling five regions of interest were designed, each spanning between 425 kb and 1,900 kb (FIG. 6C), and pulled down the tiled regions of interest with 35-49% efficiency in a single step (FIG. 6D). After paired-end sequencing and normalization³⁰ (FIG. 6E), contact maps were obtained (FIG. 1A). To validate RCMC contact maps, they were compared them to high-resolution Hi-C³¹ and Micro-C¹² for the same regions. RCMC data matched both Hi-C³¹ and Micro-C¹² data at 2-kb resolution (FIG. 6F), was reproducible (FIG. 6G) and gave the expected contact frequency scaling (FIG. 7A). Thus, RCMC captures all information in target regions obtained in prior multibillion contact studies 12.31

Having validated RCMC, it was next benchmarked against other 3C datasets. Despite capturing ˜2.6-3.3 billion unique contacts, the deepest Hi-C³¹ and Micro-C¹² datasets in mESCs give sparse contact maps at fine (subkilobase) resolutions (FIG. 1B). In contrast, because RCMC focuses its sequencing reads in only regions of interest, almost all 100-bp-sized interaction bins showed at least one interaction for the most deeply sequenced region (Klf1 FIG. 1B; FIGS. 7A-E), and the RCMC maps matched genome-wide Micro-C¹² even after downsampling by ˜100-fold (FIGS. 7D-F). Indeed, with relatively modest sequencing (FIG. 7C) the genome-wide equivalent of ˜317 billion unique contacts at the Klf1 region was captured.

To visualize the improvements afforded by RCMC, contact maps were plotted comparing RCMC to Hi-C³¹ and Micro-C¹² at the five captured regions (FIGS. 8, 9A-B). While A/B-compartments, TADs, and CTCF and cohesin-mediated structural loops are well-resolved in prior high-resolution Hi-C³¹ and Micro-C¹² studies, resolving E-P interactions has proven more challenging^{8,18}. To test the ability of RCMC to resolve E-P interactions, a region around the Sox2 gene and its regulatory elements (FIG. 2A) was captured. Sox2 encodes a key pluripotency transcription factor, whose expression in mESCs is controlled by a well-characterized ˜100-kb distal enhancer (Sox2 control region (SCR))³²⁻³⁴. Although long-range Sox2-SCR interactions are visible in Hi-C and Micro-C, RCMC resolved the fine-scale substructure of the Sox2-SCR interactions; rather than one broad loop, Sox2 forms multiple individual focal interactions with subelements of the SCR marked by Mediator binding and ATAC peaks (FIG. 2A). Furthermore, RCMC also revealed previously unobservable long-range interactions between a ˜600-700 kb distal region near the Fxr1 gene and Sox2 and the SCR as well as strong compartmental exclusion of a ˜550-kb intervening region (FIG. 9A). Next studies focused on a ˜300-kb segment of the most deeply sequenced region, the region around Klf1 (FIG. 2B). Notably, RCMC revealed patterns of highly focal and nested interactions in the Klf1 region that are not visible in genome-wide Hi-C or Micro-C data (FIG. 2B). These interactions are named microcompartments (see Discussion for rationale and definition). In conclusion for mapping genomic interactions within specific regions, RCMC outperforms genome-wide Hi-C and Micro-C at a fraction of the cost.

Related methods Micro-Capture-C (MCC)¹⁶ and Tiled-Micro-Capture-C (TMCC)¹⁷ have been reported. Unlike RCMC, (T) MCC uses only formaldehyde for fixation³⁵, skips the pull-down of ligation products and the gel purification of dinucleosomes (FIG. 1A) and instead uses sonication to generate small fragments containing both ligated and unligated DNA. This allows (T) MCC to precisely sequence the ligation junction, which for RCMC requires longer-read sequencing. Thus, this affords (T) MCC base-pair resolution when capturing the interactions between regulatory elements^{16,17}. However, by not enriching for the informative ligation products, (T) MCC mainly captures unligated DNA fragments, resulting in most sequencing reads being uninformative (FIG. 1B). Indeed, with only slightly deeper sequencing, RCMC captured ˜200 million unique 1-kb cis contacts in the target regions compared to just ˜9-13 million for TMCC, underscoring the more than one order of magnitude higher efficiency of RCMC (FIG. 7C). To directly compare RCMC to TMCC, probes were designed against the same Nanog region used in TMCC¹⁷. Due to the less efficient nature of TMCC, even with almost four-fold higher sequencing at the Nanog region, TMCC maps were noisier than RCMC, which became even more evident when the TMCC's sequencing depth was subsampled to match RCMC (FIG. 2C and FIG. 9B). In summary, the data establishes that RCMC is more efficient for general 3D genome structure mapping of a region, whereas (T) MCC may be applied when it is necessary to resolve ligation junctions with base-pair resolution.

### RCMC Reveals Nested Focal Interactions in Gene-Rich Regions

RCMC data revealed highly nested and focal interactions in both the Klf1 and Ppm1g regions, which were not visible in multibillion contact genome-wide Hi-C³¹ and Micro-C¹² datasets (FIGS. 2B, 3A,3B and FIG. 10A, 10BExisting loop^{36,37} and compartment calling algorithms³⁶ were applied to identify these interactions, but they did not reliably detect them (FIG. 10C). 132 anchors forming a total of 1,091 focal interactions in the gene-rich Klf1 and Ppm1g regions were manually identified (FIGS. 3A-B, and FIG. 10D). Furthermore, its validated that these interactions were not due to incomplete contact map normalization³⁰ (FIGS. 6E and 11A) nor an artifact of increased accessibility at the anchors (only about half of all ATAC peaks result in ‘dots,’ and not all dots are anchored by ATAC peaks; FIG. 11B-11D).

Next, it was observed that these interactions resemble both loops and compartments. Like loops, they give rise to focal enrichments (dots in FIGS. 3A-B) between two anchors and occasionally form contact domains as small as a few kilobases (squares in FIGS. 3A-B). Like A/B-compartments, they result in nested, tessellated interactions in a checkerboard-like fashion, with a mean of ˜17 interactions per anchor (mean interaction length: ˜240 kb) and the most nested anchor forming 52 focal interactions (FIGS. 3C-D). Because these highly nested and focal interactions (dots) resemble fine-scale compartmental interactions (Discussion), they are referred to herein as microcompartments.

To understand which genomic elements form microcompartments, the chromatin states of microcompartment anchors was investigated (FIGS. 3C, and FIG. 12). About two-thirds of the identified microcompartment anchors overlapped either promoter (˜46%) or enhancer (˜21%) features (FIGS. 3E, and FIG. 12), with the remaining anchors either corresponding to CTCF and cohesin-bound anchors or unknowns (Other). Notably, however, promoters and enhancers formed many more focal interactions (FIG. 3F). Specifically, promoters and enhancers formed a mean of 24 and 18 interactions, respectively, compared to just 5.5 and 7.4 for CTCF and cohesin, and ‘other’ anchors, respectively (FIG. 3F). Indeed, 74% of all annotated microcompartmental dots represented either P-P or E-P interactions, whereas only 4% of interactions were between anchors which exclusively overlapped CTCF and cohesin (FIG. 3G). Taken together, these observations suggest that microcompartments largely represent nested interactions between promoter and enhancer regions as well as some currently poorly understood ‘other’ regions.

### Most Microcompartments are Robust to Loss of Loop Extrusion

Having identified microcompartments as nested interactions frequently linking enhancers and promoters (FIGS. 3A-B), advantage was taken of the cost-effective nature of RCMC to test the roles of loop extrusion and transcription (below) in forming these interactions.

First, the role of cohesin and cohesin-mediated loop extrusion was explored. Acute loss of cohesin strengthens large-scale A/B-compartments while simultaneously causing the global loss of TADs, loop domains, and CTCF and cohesin-mediated structural loops^{13,21,24,25,27,38}. Therefore, to understand whether cohesin regulates microcompartments, a previously validated mESC line was used to acutely deplete the cohesin subunit RAD21 (mESC clone FIM RAD21-mAID-BFP-V5)^{13,38} and performed RCMC across all five regions with and without 3 h of cohesin depletion (FIGS. 4A, and FIG. 13A). The cohesin depletion was ˜97% efficient (FIG. 4B), diminished the well-characterized CTCF and cohesin-mediated Fbn2 loop³⁸ (FIG. 13A), led to the expected change in contact frequency^{21,23,24} (FIG. 13B), and was reproducible between replicates (FIGS. 16A-16E), thus validating the cohesin depletion. As expected, the small fraction of interactions between CTCF and cohesin-bound sites showed large reductions in strength upon cohesin depletion (FIGS. 4A,C, and FIG. 13A). However, the strengths of microcompartmental interactions, including E-P, E-E, and P-P^{13,17} were largely unaffected by cohesin depletion (FIG. 4C). Specifically, though clear individual examples of especially P-P interactions that either slightly strengthen are seen (FIGS. 4E, 4I) or strongly weaken (FIG. 4E, ii-iii) after cohesin depletion (FIGS. 4D and 4E), most microcompartmental interactions were largely unaffected (FIG. 4C). The definition of microcompartment was refined to “interactions largely robust to cohesin depletion (see Discussion for full definition)”.

### Most Microcompartments are Robust to Loss of Transcription

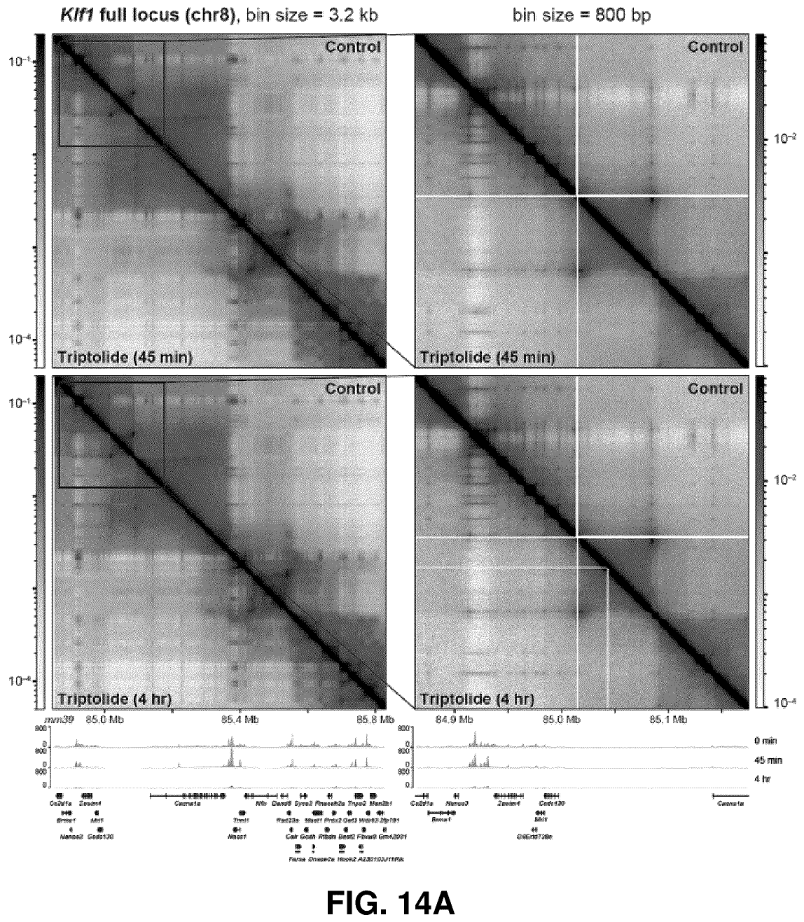

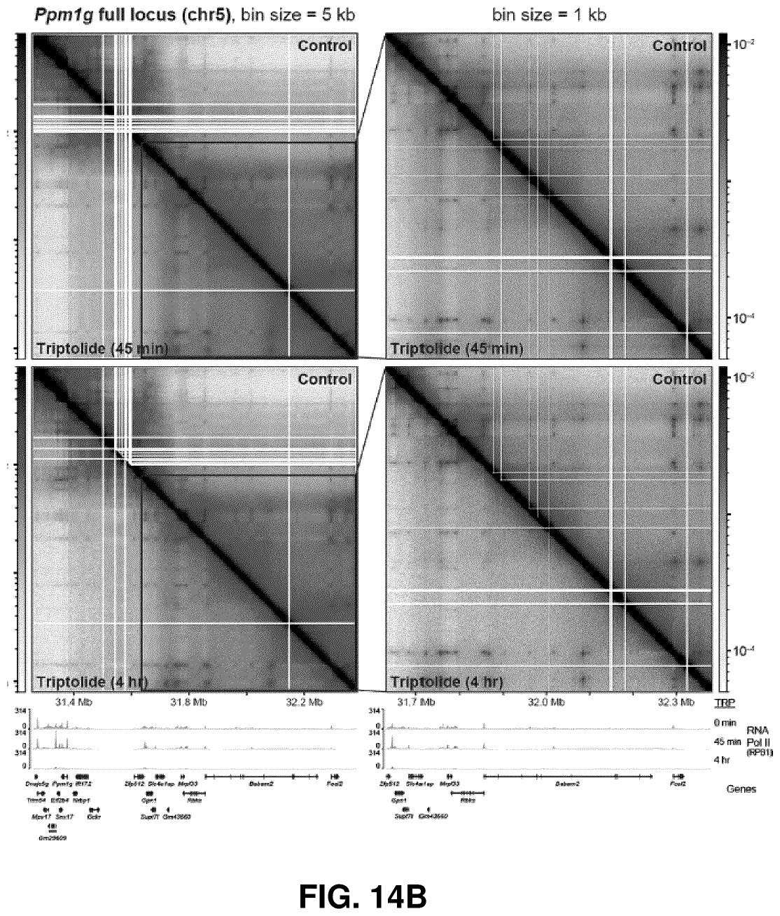

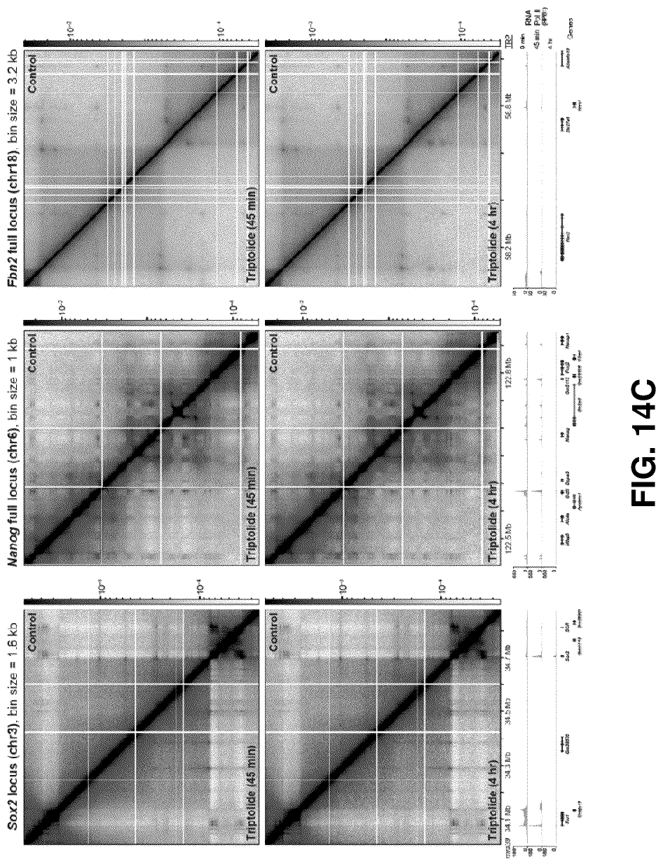

Second, the role of transcription was explored. It was observed that microcompartments are largely formed between active promoter and enhancer regions (FIGS. 3E, 3G, and FIG. 12), suggesting a relationship between active transcription and microcompartments. To understand if microcompartments are a downstream consequence of transcription, transcription was abolished by inhibiting transcription initiation by RNA polymerase II (Pol II) using triptolide. Two timepoints were chosen: 45 min, which was previously reported to modestly affect global E-P and P-P stripes¹², and 4 h, which was recently reported to greatly reduce punctate H3K4me3 (found at active promoters) and H3K27ac (found at active enhancers) marks in mESCs in addition to inhibiting transcription³⁹. RCMC was performed across all five captured regions and ChIP-seq gave the expected reduction of RNA Pol II signal, with the 4 h triptolide treatment more thoroughly eliminating RNA Pol II at promoters and throughout gene bodies (FIGS. 5A, 5B; FIG. 14). It was observed both weakened and strengthened E-P and P-P interactions (FIG. 5C-E), as well interesting dynamically changing interactions (for example, FIG. 5C (i) increases in strength with 45 min of triptolide treatment but then weakens after 4 h). Nevertheless, the strong majority of microcompartmental interactions were largely unaffected by the inhibition of transcription (FIGS. 5A, C-E). The findings herein differ somewhat from recent studies reporting global weakening of E-P interactions after 14 h of ˜80% depletion of RNA Pol II²⁰ or inhibition¹⁹. In addition to differences in cell type, treatment and treatment length, this difference may be due to the much lower depth (˜1-1.7 billion Micro-C contacts)^{19,20} used in these studies, which cannot resolve microcompartmental interactions and fine-scale E-P and P-P interactions (FIG. 3A, 5B, and FIGS. 7E, 7F). Alternatively, because microcompartments are only observed in the very gene-dense Klf1 and Ppm1g regions, prior findings^{12,19,20} may apply more to individual, isolated E-P/P-P interactions instead of dense and nested microcompartments.

In summary, microcompartments generally do not require transcription at short timescales and are more likely either independent from or formed upstream of transcription rather than forming as a downstream consequence of transcription.

### DISCUSSION

Here RCMC is introduced as an accessible and affordable method for mapping 3D genome structure at unprecedented depth. Compared with Micro-Capture-C¹⁶ methods such as TMCC¹⁷, RCMC is much more efficient (FIGS. 1B, and FIG. 7C), thus affording much higher depth with less sequencing. Another approach is to use brute-force genome-wide Hi-C or Micro-C; by performing 150 separate Hi-C experiments and sequencing deeper than ever before, a recent study by Harris et al. reached 33 billion contacts¹⁴. However, such efforts¹⁴ are expensive, not accessible to most labs, and poorly compatible with perturbation experiments vital to uncovering mechanisms of organization. Instead, RCMC reaches the local equivalent of 317 billion contacts with relatively modest sequencing (FIG. 7C). Thus, RCMC is an ideal method for generating ultra-deep 3D contact maps and for perturbation experiments.

What molecular processes might drive microcompartment formation? Although cohesin-mediated loop extrusion is well established to generate focal interactions (loops) 9.10, microcompartmental loops are largely robust to acute cohesin removal and therefore likely not dependent on loop extrusion (FIGS. 4A, and 4C). Furthermore, although most microcompartmental loops connect enhancers and promoters, microcompartments are also generally robust to the acute loss of RNA Pol II transcription initiation (FIGS. 5A and 5E). Instead, possibly, nested, multiway, and focal microcompartments correspond to small, punctate A-compartments^{14,40,41} that form through a compartmentalization mechanism, perhaps mediated by factors upstream of RNA Pol II initiation such as transcription factors and co-factors or active chromatin states⁴². Indeed, in the field of polymer physics, it is well established that block copolymers undergo microphase separation^{4,43-45} when composed of distinct monomers that preferentially self-interact (FIG. 5F). Intuitively, if active chromatin regions at microcompartment anchors are selectively ‘sticky’ with each other, they will tend to co-segregate, resulting in the formation of nested, focal interactions (FIG. 5F). Microphase separation due to preferential interactions among active loci within a block copolymer might thus explain the formation of the striking pattern of interactions observed (FIGS. 3A, 3B and 5F). In summary, microcompartments are defined as (1) highly nested, focal interactions that frequently connect promoters and enhancer regions often in gene-rich loci; (2) formed through a compartmentalization mechanism; and (3) for the most part independent of loop extrusion and transcription, at least on short timescales.

How do microcompartments compare to previously described 3D genome features? First, previous genome-wide Micro-C studies uncovered widespread short-range P-P and E-P links^{12,13}. Similarly, many microcompartmental interactions connect promoters and enhancers. RCMC now better resolves these interactions, revealing them to be highly nested, frequently forming dozens of microcompartmental loops. Second, although differences in cell type preclude a direct comparison, the microcompartments described here also share features with the fine-scale A-compartment interactions recently described by Harris et al. that were proposed to segregate active enhancers and promoters into small A-compartments¹⁴. Indeed, examining the Hi-C data of Harris et al. at 1-kb resolution reveals structures with similarities to microcompartments, suggesting that microcompartments may be conserved to human cells (FIGS. 15A-15D). Further, along the lines of Harris et al., the microcompartments observed form small contact domains, and their loops are more punctate as compared to CTCF and cohesin-mediated loops, which are more diffuse¹⁴ (FIGS. 4C and 5E).

Finally, the study disclosed herein provides insights into E-P interactions. Although some studies propose that cohesin is largely required for E-P interactions^{27,46}, others have suggested that cohesin is most important for very long-range⁴⁷⁻⁴⁹ or inducible^{48,50} E-P interactions or that cohesin is largely not required for the maintenance of E-P interactions^{13,17}. Except for some CTCF and cohesin-bound enhancers and promoters, the data herein shows that most P-P and E-P interactions are mediated by a compartmentalization mechanism distinct from loop extrusion. This may offer a mechanistic explanation for the observation that cohesin is not required for the short-term maintenance of most E-P interactions and that the effects of cohesin depletion on global gene expression are modest^{13,17,25}.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong.

### REFERENCES

1. Dekker, et al., Cell 164, 1110-1121 (2016).
2. Oudelaar, et al. Nat. Rev. Genet. 22, 154-168 (2021).
3. Lieberman-Aiden, E. et al. Science. 326, 289-293 (2009).
4. Nuebler, et al. Proc. Natl Acad. Sci. USA 115, E6697-E6706 (2018).
5. Dixon, et al. Nature 485, 376-380 (2012).
6. Nora, et al. Nature 485, 381-385 (2012).
7. Rao, et al. Cell 159, 1665-1680 (2014).
8. Goel, et al. WIREs Dev. Biol. 10, e395 (2020).
9. Sanborn, et al. Proc. Natl Acad. Sci. USA 112, 6456-6465 (2015).
10. Fudenberg, et al. Cell Rep. 15, 2038-2049 (2016).
11. Krietenstein,. et al. Mol. Cell 78, 554-565 (2020).
12. Hsieh, et al. Mol. Cell 78, 539-553 (2020).
13. Hsieh, et al. Nat. Genet. 54, 1919-1932 (2022).
14. Harris, et al. Nat. Commun. (in the press).
15. Hansen, et al. Mol. Cell 76, 395-411 (2019).
16. Hua, et al. Nature 595, 125-129 (2021).
17. Aljahani, et al. Nat. Commun. 13,2139 (2022).
18. Gasperini, et al. Cell 176, 377-390 (2019).
19. Barshad, et al. atbiorxiv.org/content/10.1101/2022.07.07.499190v1 (2022).
20 Zhang, et al. Nat. Genet. doi. org/10.1038/s41588-023-01364-4 (2023).
21 Schwarzer, et al. Nature 551, 51-56 (2017).
22 Nora, et al. Cell 169, 930-944 (2017).
23. Gassler, et al. EMBO J. 36, 3600-3618 (2017).
24. Wutz, et al. EMBO J. 36, 3573-3599 (2017).
25 Rao, et al. Cell 171, 305-320 (2017).
26. Haarhuis, et al. Cell 169, 693-707 (2017).
27 El Khattabi, et al Cell 178, 1145-1158 (2019).
28 Oudelaar, et al. Nat. Commun. 11, 2722 (2020).
29 Jäger, Nat. Commun. 6, 6178 (2015).
30. Imakaev, et al. Nat. Methods 9, 999-1003 (2012).
31. Bonev, et al. Cell 171, 557-572 (2017).
32. Zhou, et al. Genes Dev. 28, 2699-2711 (2014).
33. Li, et al. PLOS One 9, e114485 (2014).
34. Chakraborty, et al. Nat. Genet. 55, 280-290 (2023).
35. Akgol Oksuz, et al. Nat. Methods 18, 1046-1055 (2021).
36. Abdennur, et al Preprint at biorxiv.org/content/10. 1101/2022.10.31.514564v1 (2022).
37. Roayaei Ardakany, et al. Genome Biol. 21, 256 (2020).
38. Gabriele, et al. Science. 376, 496-501 (2022).
39. Wang, et al. Nat. Genet. 54, 295-305 (2022).
40. Rosencrance, et al. Mol. Cell 78, 112-126 (2020).
41. You, et al. Nat. Biotechnol. 39, 225-235 (2021).
42. Rippe, et al. Curr. Opin. Cell Biol. 74, 88-96 (2022).
43. Leibler, Macromolecules 13, 1602-1617 (1980).
44. Meier, J. Polym. Sci. Part C. Polym. Symp. 26, 81-98 (1969).
45. Fujishiro, et al. Proc. Natl Acad. Sci. USA 119, e2109838119 (2022).
46. Thiecke, et al. Cell Rep. 32, 107929 (2020).
47. Kane, et al. Nat. Struct. Mol. Biol. 29, 891-897 (2022).
48. Calderon, et al. Elife 11, e76539 (2022).
49. Rinzema, et al. Nat. Struct. Mol. Biol. 29, 563-574 (2022).
50. Cuartero, S et al. Nat. Immunol. 19, 932-941 (2018).
51. Navarro Gonzalez, et al. Nucleic Acids Res. 49, D1046-D1057 (2021).
52. Pettitt, et al. Nat. Methods 6, 493-495 (2009).
53. Hansen, et al. Elife 6, c25776 (2017).
54. Kerpedjiev, et al. Genome Biol. 19, 125 (2018).
55. Xu, et al. BMC Bioinformatics 22, 489 (2021).
56. Zhao, et al. Bioinformatics 30, 1006-1007 (2014).
57. Robinson, et al. Nat. Biotechnol. 29, 24-26 (2011).
58. Yang, et al. Genome Res. 27, 1939-1949 (2017).
59. Venev, et al. open2c/cooltools: v0.4.1 (v0.4.1). Zenodo doi.org/10.5281/zenodo.5214125 (2021).
60. Abdennur, et al. Bioinformatics 36, 311-316 (2020).
61. Robinson, et al. Cell Syst. 6, 256-258 (2018).

### TABLE 18 REFERENCES

1. Hansen A S, Pustova I, Cattoglio C, Tjian R et al. CTCF and cohesin regulate chromatin loop stability with distinct dynamics. Elife 2017 May 3; 6. PMID: 28467304
3. Hansen A S, Hsieh T S, Cattoglio C, Pustova I et al. Distinct Classes of Chromatin Loops Revealed by Deletion of an RNA-Binding Region in CTCF. Mol Cell 2019 Nov. 7; 76 (3): 395-411.e13. PMID: 31522987
3. ENCODE Project Consortium. An integrated encyclopedia of DNA elements in the human genome. Nature 2012 Sep. 6; 489 (7414): 57-74. PMID: 22955616
4. ENCODE Project Consortium. An integrated encyclopedia of DNA elements in the human genome. Nature 2012 Sep. 6; 489 (7414): 57-74. PMID: 22955616
5. ENCODE Project Consortium. An integrated encyclopedia of DNA elements in the human genome. Nature 2012 Sep. 6; 489 (7414): 57-74. PMID: 22955616
6. Chronis C, Fiziev P, Papp B, Butz. S et al. Cooperative Binding of Transcription Factors Orchestrates Reprogramming. Cell 2017 Jan. 26; 168 (3): 442-459.e20. PMID: 28111071
7. Chronis C, Fiziev P, Papp B, Butz S et al. Cooperative Binding of Transcription Factors Orchestrates Reprogramming. Cell 2017 Jan. 26; 168 (3): 442-459.e20. PMID: 28111071
8. Kagey M H, Newman J J, Bilodeau S, Zhan Y et al. Mediator and cohesin connect gene expression and chromatin architecture. Nature 2010 Sep. 23; 467 (7314): 430-5. PMID: 20720539
9. Murakami K, Günesdogan U, Zylicz J J, Tang WWC et al. NANOG alone induces germ cells in primed epiblast in vitro by activation of enhancers. Nature 2016 Jan. 21; 529 (7586): 403-407. PMID: 26751055
10. Chronis C, Fiziev P, Papp B, Butz S et al. Cooperative Binding of Transcription Factors Orchestrates Reprogramming. Cell 2017 Jan. 26; 168 (3): 442-459.e20. PMID: 28111071
11. Chronis C, Fiziev P, Papp B, Butz S et al. Cooperative Binding of Transcription Factors Orchestrates Reprogramming. Cell 2017 Jan. 26; 168 (3): 442-459.e20. PMID: 28111071
12. Chronis C, Fiziev P, Papp B, Butz S et al. Cooperative Binding of Transcription Factors Orchestrates Reprogramming. Cell 2017 Jan. 26; 168 (3): 442-459.e20. PMID: 28111071
13. Chronis C, Fiziev P, Papp B, Butz S et al. Cooperative Binding of Transcription Factors Orchestrates Reprogramming. Cell 2017 Jan. 26; 168 (3): 442-459.e20. PMID: 28111071
14. Chronis C, Fiziev P, Papp B, Butz S et al. Cooperative Binding of Transcription Factors Orchestrates Reprogramming. Cell 2017 Jan. 26; 168 (3): 442-459.e20. PMID: 28111071
15. Weintraub A S, Li C H, Zamudio A V, Sigova A A et al. YY1 Is a Structural Regulator of Enhancer-Promoter Loops. Cell 2017 Dec. 14; 171 (7): 1573-1588.e28. PMID: 29224777
16. Obri A, Ouararhni K, Papin C, Diebold M L et al. ANP32E is a histone chaperone that removes H2A.Z from chromatin. Nature 2014 Jan. 30; 505 (7485): 648-53. PMID: 24463511
17. Bonev B, Mendelson Cohen N, Szabo Q, Fritsch L et al. Multiscale 3D Genome Rewiring during Mouse Neural Development. Cell 2017 Oct. 19; 171 (3): 557-572.e24. PMID: 29053968
18. Juan A H, Wang S, Ko K D, Zare H et al. Roles of H3K27me2 and H3K27me3 Examined during Fate Specification of Embryonic Stem Cells. Cell Rep 2016 Oct. 25; 17 (5): 1369-1382. PMID: 27783950
19. Chronis C, Fiziev P, Papp B, Butz S et al. Cooperative Binding of Transcription Factors Orchestrates Reprogramming. Cell 2017 Jan. 26; 168 (3): 442-459.e20. PMID: 28111071
20. Riising E M, Comet I, Leblanc B, Wu X et al. Gene silencing triggers polycomb repressive complex 2 recruitment to CpG islands genome wide. Mol Cell 2014 Aug. 7; 55 (3): 347-60. PMID: 24999238
21. Hansen A S, Hsieh T S, Cattoglio C, Pustova I et al. Distinct Classes of Chromatin Loops Revealed by Deletion of an RNA-Binding Region in CTCF. Mol Cell 2019 Nov. 7; 76 (3): 395-411.e13. PMID: 31522987
22. King H W, Fursova N A, Blackledge N P, Klose R J. Polycomb repressive complex 1 shapes the nucleosome landscape but not accessibility at target genes. Genome Res 2018 Oct.; 28 (10): 1494-1507. PMID: 30154222
23. Hsieh T S, Cattoglio C, Slobodyanyuk E, Hansen A S et al. Enhancer-Promoter Interactions and Transcription are Maintained Upon Acute Loss of CTCF, Cohesin, WAPL, and YY1. bioRxiv 2021 Jul. 14:452-365. doi: https://doi.org/10.1101/2021.07.14.452365
24. Hsieh T S, Cattoglio C, Slobodyanyuk E, Hansen A S et al. Enhancer-Promoter Interactions and Transcription are Maintained Upon Acute Loss of CTCF, Cohesin, WAPL, and YY1. bioRxiv 2021 Jul. 14:452-365. doi: https://doi.org/10.1101/2021.07.14.452365
25. Hsieh T S, Cattoglio C, Slobodyanyuk E, Hansen A S et al. Enhancer-Promoter Interactions and Transcription are Maintained Upon Acute Loss of CTCF, Cohesin, WAPL, and YY1. bioRxiv 2021 Jul. 14:452-365. doi: https://doi.org/10.1101/2021.07.14.452365
26. Hsieh T S, Cattoglio C, Slobodyanyuk E, Hansen A S et al. Enhancer-Promoter Interactions and Transcription are Maintained Upon Acute Loss of CTCF, Cohesin, WAPL, and YY1. bioRxiv 2021 Jul. 14:452-365. doi: https://doi.org/10.1101/2021.07.14.452365
27. ENCODE Project Consortium. An integrated encyclopedia of DNA elements in the human genome. Nature 2012 Sep. 6; 489 (7414): 57-74. PMID: 22955616
28. ENCODE Project Consortium. An integrated encyclopedia of DNA elements in the human genome. Nature 2012 Sep. 6; 489 (7414): 57-74. PMID: 22955616
29. ENCODE Project Consortium. An integrated encyclopedia of DNA elements in the human genome. Nature 2012 Sep. 6; 489 (7414): 57-74. PMID: 22955616

We claim:

1. A method comprising:
(a) crosslinking genomic DNA in cells with at least two crosslinking agents to form a composition comprising crosslinked genomicDNA (cG-DNA);
(b) fragmenting the cG-DNA creating a plurality of crosslinked fragments (CFs);
(c) repairing ends of the plurality of the CFs, followed by fragment end labelling, by contacting the plurality of the crosslinked fragments with labelled nucleotides to form labelled crosslinked fragments;
(d) ligating the 1-CFs and removing labelled nucleotides from unligated fragment ends;
(e) obtaining a purified dinucleosome-sized DNA fraction in a series of steps that comprise:
(i) de-crosslinking the crosslinked fragments to obtain a mix of DNA fragments,
(ii) isolating dinucleosome-sized DNA fragments in a method comprising separation of de-crosslinked DNA fragments by size, and
(iii) isolating ligated and labelled DNA fragments in a method comprising contacting the de-crosslinked dinucleosome-sized DNA fragments with a binding partner for the label on labelled ligated fragments,
wherein each isolated dinucleosome-sized DNA fragments comprise a first and a second DNA region ligated to each other;
(f) preparing a sequencing library from the dinucleosome-sized DNA fraction and optionally amplifying the purified fragments; and
(g) performing tiling region capture of a region of interest.

2. The method of claim 1, wherein the at least two crosslinking agents are selected from the group consisting of disuccinimidyl glutarate (DSG) and formaldehyde.

3. The method of claim 1, crosslinking is performed in a single step comprising contacting genomic DNA with a first crosslinking agent for an effective amount of time to effect crosslinking, and contacting the genomic DNA with a second crosslinking agent for an effective amount of time to effect crosslinking, wherein the crosslinking is not stopped after contacting the genomic DNA with the first crosslinking agent.

4. The method of claim 1, (a) comprising counting cells following crosslinking and/or (b) wherein the step of fragmenting the crosslinked genomic DNA is not carried out by restriction enzymes or sonication.

5. The method of claim 1, wherein the step of fragmenting the crosslinked genomic DNA comprises contacting the crosslinked genomic DNA composition with a micrococcal nuclease (MNase) composition in an amount and a time effective for digestion of the chromatin.

6. The method of claim 5, wherein the fragmenting step produces nucleosome-sized fragments (about 150-200 bp) fragments.

7. The method of claim 1, wherein the labelling agent is biotin, wherein the nucleosome-sized fragments are end-labelled by contacting with a pool of biotin-labelled nucleotides.

8. The method of claim 1, wherein the step of de-crosslinking comprises adding NaCl (about 10-about 250 mM) to a de-crosslinking reaction mixture.

9. The method of claim 1, wherein the method steps do not include an ethanol precipitation (of DNA) step.

10. The method of claim 1 wherein size selection is performed using gel electrophoresis and extraction.

11. The method of claim 1 comprising isolating ligated dinucleosome DNA fragments from the de-crosslinked reaction mixture in a method comprising contacting label-bound nucleosome-sized fragments with a binding partner for the label, optionally, wherein the label is biotin and the binding partner is stretavidin.

12. The method of claim 1, wherein the first region is a promoter region and the second region comprises a regulatory sequence being located close or distantly from each other in the DNA or on the same or different chromosomes.

13. The method of claim 12, wherein said second region comprises an enhancer sequence.

14. The method of claim 12, wherein the regulatory sequence is an enhancer, silencer, or insulator.

15. The method of claim 12, wherein the 3D interactions of any genomic region of interest with another region is studied.

16. The method of claim 1, comprising one or more steps of: (i) end polishing, (ii) streptavidin purification, (iii) end repair & A-tailing, (iv) adapter ligation, (vi) bead washing, (vii) test PCR run, (ix) pool PCR run, (x) sample purification, (xi) sample quantification, and (xii) pooling of barcoded samples.

17. The method of claim 1, wherein tiling region capture of a region of interest comprises adding labelled capture probes, wherein the labelled capture probes hybridize to regulatory regions of the crosslinked genomic DNA; selectively purifying the fragments that hybridize to the labelled capture probes and analyzing the fragments that hybridize to the labelled capture probes to determine the identity of the fragments.

18. The method of claim 17, wherein the labelled capture probes are biotin-labelled oligonucleotides.

19. The method of claim 1, wherein the method identifies microcompartments.

20. The method of claim 1, wherein the cG-DNA is fragmented using MNase digestion or DNase digestion.
